(19) United States
(12) Patent Application Publication
(10) Pub. No.: US 2025/0120943 A1
(43) Pub. Date: Apr. 17, 2025

(54) RIFAMYCIN OPHTHALMIC COMPOSITION AND USE THEREOF

(71) Applicants: AMD THERAPEUTICS LLC, Palo Alto, CA (US); BNC KOREA, INC., Dalseo-gu (KR); TEIKA PHARMACEUTICAL CO., LTD., Toyama-shi (JP)

(72) Inventors: Hiroaki SERIZAWA, Mountain View, CA (US); Naohisa WADA, Toyama (JP)

(73) Assignees: AMD THERAPEUTICS LLC, Palo Alto, CA (US); BNC KOREA, INC., Dalseo-gu (KR); TEIKA PHARMACEUTICAL CO., LTD., Toyama-shi (JP)

(21) Appl. No.: 18/966,037

(22) Filed: Dec. 2, 2024

Related U.S. Application Data
(63) Continuation of application No. PCT/JP2023/021073, filed on May 31, 2023.

(30) Foreign Application Priority Data
Jun. 3, 2022 (JP) 2022-090997
Feb. 7, 2023 (JP) 2023-017037

(51) Int. Cl. A61K 31/395; A61K 9/00; A61K 47/38; A61K 47/44; A61P 27/02

(52) U.S. Cl. CPC A61K 31/395; A61K 9/0048; A61K 47/38; A61K 47/44; A61P 27/02

(57) ABSTRACT

Provided herein are ophthalmic compositions comprising an effective amount of one or more rifamycin compounds or a pharmaceutically acceptable salt thereof, methods for preparing said compositions, and methods for use of said compositions in the treatment of various disorders.

### TECHNICAL FIELD

The present technology relates to ophthalmic compositions comprising an effective amount of one or more rifamycin compounds or a pharmaceutically acceptable salt thereof, methods for preparing said compositions, and methods for use of said compositions in the treatment of various disorders.

### BACKGROUND

The following description of the background of the present technology is provided simply as an aid in understanding the present technology and is not admitted to describe or constitute prior art to the present technology.

Loss of visual acuity is a common problem associated with aging, or with various diseases of eyes, such as macular degeneration, ocular histoplasmosis syndrome, myopia, diabetic retinopathy and inflammatory disease, all of which are caused by neovascularization in the cornea, retina or choroid.

Age-related macular degeneration (AMD) is a common eye disease, which usually affects elderly people and brings on a loss of vision in the center of the visual field (the macula) due to retinal damage. Although some peripheral vision remains, it is difficult or impossible to read or recognize faces. There are two major forms of macular degeneration, namely, atrophic (dry) and exudative (wet) forms. In dry (non-exudative) form, cellular debris called “drusen” accumulates between retina and choroid. In a more severe wet (exudative) form, blood vessels grow up from the choroid behind the retina. AMD is a leading cause of blindness among people older than 65 years and is caused by abnormal development of blood vessels behind retina. The advanced AMD population will increase by 11% and will reach 3.3 million due to the aging population. Intravitreal injection with anti-vascular endothelial growth factor (anti-VEGF) therapy has become the criterion standard for treatment of choroidal neovascular membranes (CNVs) associated with AMD.

Treatment options for wet AMD include bevacizumab (Avastin, Genentech, San Francisco, CA), which is a full-length anti-VEGF antibody, ranibizumab (Lucentis, Genentech), which is an affinity-matured fragment, pegaptanib sodium (Macugen, OSI/Eyetech Inc.), and aflibercept (Eylea, Regeneron, Tarrytown, NY), and other anti-VEGF drugs. However, such intravitreal injection is a process that requires high precision, because it is performed with the help of a needle under local anesthesia. In this process, the needle must be inserted into the vitreous liquid that fills the cavity between the lens and retina, and thus, the operation must be carried out very carefully not to damage the retina. Accordingly, there is an urgent need for improved treatment methods, administration routes, and methods for treating ocular disorders such as AMD. The present disclosure addresses this urgent need, and provides formulations capable of efficient delivery of one or more rifamycin compounds to the eye, particularly the sub-retina and sub-sclera.

### SUMMARY OF THE INVENTION

The present disclosure generally relates to ophthalmic compositions comprising, or consisting essentially of, or consisting of an effective amount of one or more rifamycin compounds or a pharmaceutically acceptable salt thereof, and one or more viscosity imparting agents. The present disclosure also provides methods for preparing said compositions. The compositions and methods of the present disclosure are useful for the treatment of various disorders, particularly, various disorders of the eye.

Accordingly, in one aspect, the disclosure provides an ophthalmic composition having a viscosity of at least 1 mPaS at 25° C., the composition comprising, or consisting essentially of, or consisting of: a) an effective amount of one or more rifamycin compounds or a pharmaceutically acceptable salt thereof, b) a buffer solution, and c) one or more viscosity imparting agents.

In certain embodiments, the one or more rifamycin compounds is selected from the group of rifamycin SV, 3-formyl rifamycin SV, rifampicin, rifabutin, rifapentine, or rifaximin. In a further embodiment, the one or more rifamycin compounds is rifampicin. In some embodiments, the effective amount of the one or more rifamycin compounds comprises, or consists essentially of, or consists of a final concentration in the composition of at least 0.001 to at least 0.01% w/w, or at least 0.01 to at least 0.25% w/w, or at least 0.25 to at least 0.5% w/w, or 0.5 to at least 1.5% w/w, or at least 0.75 to at least 1.5% w/w, or at least 1 to at least 1.5% w/w, or at least 1.5% w/w.

In certain embodiments, the buffer solution is selected from the group of: acetic acid, boric acid, citric acid, lactic acid, phosphoric acid, hydrochloric acid-potassium chloride, glycine, aconitic acid, citric acid-phosphoric acid, succinic acid, phthalic acid, maleic acid, cacodylic acid, tris (trishydroxymethylaminomethane), barbituric acid, borax, 2-amino-2-methyl-1,3-propanediol (Ammediol), sodium carbonate-sodium bicarbonate, HEPES (4-(2-hydroxyethyl)-1-piperazineethanesulfonic acid), ACES (N-(2-acetamido)-2-aminoethanesulfonic acid), ADA (N-(2-acetamido) iminodiacetic acid), BES (N,N-bis(2-hydroxyethyl)-2-aminoethanesulfonic acid), Bicine (N,N-bis(2-hydroxyethyl) glycine), Bis-Tris (bis(2-hydroxyethyl) iminotris(hydroxymethyl) methane), CAPS (N-cyclohexyl-3-aminopropanesulfonic acid), CAPSO (N-cyclohexyl-2-hydroxy-3-aminopropanesulfonic acid), CHES (N-cyclohexyl-2-aminoethanesulfonic acid), DIPSO (3-[N,N-bis(2-hydroxyethyl)amino]-2-hydroxypropanesulfonic acid), EPPS (3-[4-(2-hydroxyethyl)-1-piperazinyl] propanesulfonic acid), HEPES-Na (sodium 2-[4-(2-hydroxyethyl)-1-piperazinyl] ethanesulfonate), HEPPSO (2-hydroxy-3-[4-(2-hydroxyethyl)-1-piperazinyl] propanesulfonic acid, monohydrate), MES (2-morpholinoethanesulfonic acid, monohydrate), MOPS (3-morpholinopropanesulfonic acid), MOPSO (2-hydroxy-3-morpholinopropanesulfonic acid), PIPES (piperazine-1,4-bis(2-ethanesulfonic acid)), POPSO (piperazine-1,4-bis(2-hydroxy-3-propanesulfonic acid), dihydrate), TAPSO (2-hydroxy-N-tris(hydroxymethyl)methyl-3-aminopropanesulfonic acid), TES (N-tris(hydroxymethyl)methyl-2-aminoethanesulfonic acid), Tricine (N-[tris(hydroxymethyl)methyl]glycine), hydrochloric acid, sodium hydroxide, sodium phosphate, sodium borate, sodium citrate, sodium acetate, and sodium lactate; and buffer agents, such as citrate/dextrose, sodium bicarbonate, and ammonium chloride, and citrate, phosphate, borate, bicarbonate, sodium salt, or potassium.

In certain embodiments, the one or more viscosity imparting agents is selected from the group of: petrolatum, liquid paraffin, light liquid paraffin, castor oil, mineral oil, cotton seed oil, soybean oil, sesame oil, corn oil, Petroleum resin, macrogol, glycerol, polybutene, rosin, polyvinyl alcohol, polystyrene, polyacrylic acid, propylene glycol, piperonyl butoxide, hypromellose, talc, gelatin, hydrogenated rosin glycerol ester, aliphatic hydrocarbon resin, benzyl acetate, copal resin, silicic acid, silicone, dimethylpolysiloxane, aluminum magnesium silicate, xanthan gum, sodium chondroitin sulfate, cyclodextrin, carboxyvinyl polymer, sodium alginate, propylene glycol alginate, carrageenan, carmellose sodium, gluconolactone, squalene, stearyl alcohol, aluminum stearate, lanolin, cetanol, gelatin, sorbitol, dextran, dextrin, tragacanth, palmitic acid, hyaluronate, hydroxyethylcellulose, hydroxyethylmethylcellulose, hydroxypropylcellulose, butylene glycol, polyoxyethylene polyoxypropylene glycol, polysorbate, sodium metaphosphate, methylcellulose, methyl vinyl ester maleic anhydride copolymer, locust bean gum, or cellulosic polymers. In some further embodiments, the one or more viscosity imparting agents is petrolatum or liquid paraffin.

In certain embodiments, the composition comprises, or consists essentially of, or consists of a final viscosity of at least about 1 mPaS to at least about 4000 mPaS, about 0 mPaS to at least about 1 mPaS at 25° C., at least about 1 mPaS to at least about 5 mPaS at 25° C., at least about 5 mPaS to at least about 50 mPaS at 25° C., at least about 50 mPaS to at least about 100 mPaS at 25° C., or at least about 100 mPaS to at least about 200 mPaS at 25° C., or optionally about 100 mPaS, or about 150 mPaS, or about 160 mPaS, or about 170 mPaS, or about 180 mPaS, or about 190 mPaS, or about 200 mPaS, at 25° C. In some further embodiments, the composition comprises, or consists essentially of, or consists of a final viscosity of at least about 161 mPaS at 25° C.

In certain embodiments, the composition comprises, or consists essentially of, or consists of a final viscosity of at least about 500 mPaS to at least about 900 mPaS at 25° C., or optionally about 500 mPaS, about 600 mPaS, about 700 mPaS, about 800 mPaS, about 850 mPaS, about 860 mPaS, about 870 mPaS, about 880 mPaS, about 890 mPaS, or about 900 mPaS, at 25° C. In some further embodiments, the composition comprises, or consists essentially of, or consists of a final viscosity of at least about 867 mPaS at 25° C.

In certain embodiments, the composition comprises, or consists essentially of, or consists of a final viscosity of at least about 1000 mPaS to at least about 2500 mPaS at 25° C., or optionally about 1000 mPaS, about 1500 mPaS, about 2000 mPaS, about 2100 mPaS, about 2200 mPaS, about 2300 mPaS, about 2400 mPaS, or about 2500 mPaS, at 25° C. In some further embodiments, the composition comprises, or consists essentially of, or consists of a final viscosity of at least about 2145 mPaS, at 25° C.

In certain embodiments, the composition comprises, or consists essentially of, or consists of a viscosity of at least about 3000 mPaS to at least about 4000 mPaS at 25° C., or optionally about 3000 mPaS, about 3500 mPaS, about 3600 mPaS, about 3700 mPaS, about 3800 mPaS, about 3900 mPaS, or about 4000 mPaS, at 25° C. In some further embodiments, the composition comprises, or consists essentially of, or consists of a viscosity of at least about 3815 mPaS at 25° C.

In certain embodiments, the one or more rifamycin compounds is rifampicin, the one or more viscosity imparting agents is petrolatum, and the effective amount of rifampicin comprises, or consists essentially of, or consists of a final concentration in the composition of at least about 0.001% w/w. In some embodiments, the one or more rifamycin compounds is rifampicin, the one or more viscosity imparting agents is liquid paraffin, and the effective amount of rifampicin comprises a final concentration in the composition of at least about 0.001% w/w to about 1% w/w. In some embodiments, the one or more rifamycin compounds is rifampicin, the one or more viscosity imparting agents is petrolatum, and the effective amount of rifampicin comprises a final concentration in the composition of at least about 0.001% w/w to about 1% w/w. In some embodiments, the one or more rifamycin compounds is rifampicin, the one or more viscosity imparting agents is liquid paraffin, and the effective amount of rifampicin comprises a final concentration in the composition of at least about 0.001% w/w to about 1% w/w.

In one embodiment, the effective amount of one or more rifamycin compounds or a pharmaceutically acceptable salt thereof is the only therapeutically effective compound in the composition.

In certain embodiments, the disclosure provides a method for delivering one or more rifamycin compounds to the sub retina, sclera, retina, and/or vitreous tissues, the method comprising, or consisting essentially of, or consisting of topically administering any one of the compositions disclosed herein to the eye.

In certain embodiments, the disclosure provides a method for treating a neovascular eye disease, the method comprising, or consisting essentially of, or consisting of topically administering any one of the compositions disclosed herein to the eye. In some embodiments of the method, the neovascular eye disease is selected from the group consisting of macular degeneration, diabetic retinopathy, chronic glaucoma, retinal detachment, sickle cell retinopathy, age-related macular degeneration (AMD), retinal ganglion cell injury, rubeosis iritis, inflammatory disease, chronic uveitis, neoplasm, Fuchs' heterochromic iridocyclitis, neovascular glaucoma, corneal neovascularization, choroidal neovascularization, retinal neovascularization, retinal angiomatous proliferation, glaucoma, glaucoma surgery, tissue adhesion, cicatrization, tissue fibrosis, and brain damage. In some further embodiments, the neovascular eye disease is AMD. In some further embodiments, the neovascular eye disease is dry AMD. In some further embodiments, the neovascular eye disease is wet AMD. In some further embodiments, the neovascular eye disease is diabetic retinopathy.

In some further embodiments, administration of the composition results in delivery of the one or more rifamycin compounds to the sub retina, sclera, retina, and/or vitreous tissues. In some further embodiments, administration of the composition results in delivery of the one or more rifamycin compounds to the sub retina and sclera. In some further embodiments, administration of the composition inhibits neovascularization in sub-retina tissues.

In some further embodiments, topical administration of the composition results in at least about a 5-fold, 10-fold, 50-fold, or 100-fold reduction in plasma exposure of the one or more rifamycin compounds relative to oral dosing at 300 mg. In some embodiments, topical administration of the composition results in at least about 100-fold reduction in plasma exposure of the one or more rifamycin compounds relative to oral dosing at 300 mg.

### BRIEF DESCRIPTION OF THE DRAWINGS

FIGS. 1A to 1H are histology sections of retinas treated with a rifampicin topical eye drop formulation, a rifampicin SC injection, and a control of only vehicle, in which oxygen-induced retinopathy was induced in the retinas, and a retina in which retinopathy was not induced; and histology sections of retinas treated with a rifampicin topical eye drop formulation, a rifampicin SC injection, and a control of only vehicle, in which oxygen-induced retinopathy was induced in the retinas; and a retina in which retinopathy was not induced. FIG. 1A shows the 200× histological section of a retina treated with a control of only vehicle. FIG. 1B shows the 400× histological section of a retina treated with a control of only vehicle. FIG. 1C shows the 200× histological section of a retina treated with a rifampicin topical eye drop formulation. FIG. 1D shows the 400× histological section of a retina treated with a rifampicin topical eye drop formulation. FIG. 1E shows the 200× histological section of a retina treated with a rifampicin SC injection. FIG. 1F shows the 400× histological section of a retina treated with a rifampicin SC injection. FIG. 1G shows the 200× histological section of a retina, in which retinopathy was not induced. FIG. 1H shows the 400× histological section of a retina, in which retinopathy was not induced.

FIGS. 2A and 2B are graphs showing representative AUC (X-Fold AUC) vs. viscosity data (mPaS). AUC values evaluated in sub-retina and retina tissues in pharmacokinetics studies were analyzed to determine AUC correlations with the viscosities of Formulations A-F as described in Example 12. AUC (as X-Fold AUC) was plotted against the viscosity (mPaS) for each dose administered. FIG. 2A shows the AUC vs. viscosity data for delivery to sub-retina tissue. FIG. 2B shows AUC vs. viscosity data for delivery to retina tissue.

FIG. 3A and FIG. 3B are graphs showing the relationship between viscosity and shear velocity for oil-based formulations (A-F) and water-based formulations (G, H, and RK32) at 25° C. The relationship between viscosity (mPaS) and shear velocity (s⁻¹) for each formulation is depicted in the viscosity range of 0 mPaS to 200,000 mPaS (FIG. 3A) and 0 mPaS to 100,000 mPaS (FIG. 3B). Oil-based Formulations A-E exhibited a shear velocity of 200 s⁻¹. Water-based formulations G, H, and RK32 exhibited a shear velocity of 200 s⁻¹.

FIG. 4 shows the chemical structures of exemplary suitable rifamycin compounds of the present disclosure. Two views of rifamycin SV are provided.

### DETAILED DESCRIPTION

It is to be appreciated that certain aspects, modes, embodiments, variations and features of the present methods are described below in various levels of detail in order to provide a substantial understanding of the present technology.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

The present disclosure demonstrates that administration of the ophthalmic compositions and formulations described herein deliver one or more rifamycin compounds to the back of the eye more efficiently than previous formulations. As demonstrated in the Examples herein, administration of the ophthalmic compositions and formulations of the present disclosure results in delivery of one or more rifamycin compounds to the back of the eye at concentrations effective to inhibit neovascularization. Accordingly, the disclosure provides improved compositions and formulations capable of achieving more efficient delivery of one or more rifamycin compounds to the eye. The improved compositions and formulations of the present technology are useful in methods for treating various ophthalmic diseases.

### Definitions

Unless defined otherwise, all technical and scientific terms used herein have the meaning commonly understood by a person skilled in the art to which this disclosure belongs. As used herein, the following terms have the meanings ascribed to them below, unless specified otherwise. The terminology used herein is for the purpose of describing particular embodiments only and is not intended to be limiting of the disclosure.

As used herein, the singular forms “a”, “an” and “the” are intended to include the plural forms as well, unless the context clearly indicates otherwise.

Unless otherwise indicated, all numbers expressing quantities of ingredients, reaction conditions, and so on, which are used in the description and the claims are to be understood as being modified in all instances by the term “about”. As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value.

Combinations of substituents and variables are only those that result in the formation of stable compounds. The term “stable”, when used herein, refers to compounds, which possess stability sufficient to enable production, and which maintains the integrity of the compounds for a sufficient period of time, so that the compounds are useful for the purposes detailed herein (for example, 2 hours, 5 hours, 10 hours, whole day and night, 1 day, 2 days, 3 days, 1 week, 2 weeks, 3 weeks, 1 month, 2 months, 3 months, 6 months, 1 year, 2 years, and 3 years).

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a composition known to exhibit the desired therapeutic effect) and a negative control (a subject or a sample that does not receive the therapy or receives a placebo) are typically employed.

As used herein, the term “effective amount” or “therapeutically effective amount” refers to a quantity of an agent sufficient to achieve a desired therapeutic effect. In the context of therapeutic applications, the amount of a therapeutic agent administered to the subject can depend on the type and severity of the infection and on the characteristics of the individual, such as general health, age, sex, body weight and tolerance to drugs. It can also depend on the degree, severity and type of disease. The skilled artisan will be able to determine appropriate dosages depending on these and other factors.

The terms “carrier” and “vehicle”, when used herein, refer to relatively nontoxic chemical compounds or agents that facilitate the incorporation of a compound into cells, for example, ocular cells, or tissues. Carriers and vehicles useful herein include any given materials known in the present technical field, which are nontoxic and do not interact with other components of the formulation in a harmful manner. When used in the present description, the term “pharmaceutically acceptable carrier” includes any given excipients, and all solvents, dispersion media, coatings, wetting agents (e.g., sodium lauryl sulfate), isotonic and absorption delaying agents, disintegrants (e.g., potato starch or sodium starch glycolate), and tonicity adjusting agents.

Examples of an excipient of the present disclosure includes one or more “viscosity imparting agents”. As used herein, “viscosity imparting agents” refers to one or more relatively nontoxic chemical compounds or agents that change the viscosity of pharmaceutical ingredients and/or formulations. Representative examples of viscosity imparting agents include petrolatum, liquid paraffin, light liquid paraffin, castor oil, mineral oil, cotton seed oil, soybean oil, sesame oil, corn oil, Petroleum resin, macrogol, glycerol, polybutene, rosin, polyvinyl alcohol, polystyrene, polyacrylic acid, propylene glycol, piperonyl butoxide, hypromellose, talc, gelatin, hydrogenated rosin glycerol ester, aliphatic hydrocarbon resin, benzyl acetate, copal resin, silicic acid, silicone, dimethylpolysiloxane, aluminum magnesium silicate, xanthan gum, sodium chondroitin sulfate, cyclodextrin, carboxyvinyl polymer, sodium alginate, propylene glycol alginate, carrageenan, carmellose sodium, gluconolactone, squalene, stearyl alcohol, aluminum stearate, lanolin, cetanol, gelatin, sorbitol, dextran, dextrin, tragacanth, palmitic acid, hyaluronate, hydroxyethylcellulose, hydroxyethylmethylcellulose, hydroxypropylcellulose, butylene glycol, polyoxyethylene polyoxypropylene glycol, polysorbate, sodium metaphosphate, methylcellulose, methyl vinyl ester maleic anhydride copolymer, locust bean gum, cellulosic polymers, etc.

Other representative examples of excipients of the present disclosure include antioxidants (thiosulfate, sodium thiosulfate, sodium formaldehyde sulfoxylate, sodium formaldehyde sulfoxylate dihydrate, etc.), and tonicity adjusting agents (sodium chloride, etc.).

As used herein, the term “surfactant” refers to any given molecule having both a polar head group, which energetically prefers solvation by water, and a hydrophobic tail, which is not well solvated by water. The surfactant can be an ionic or a nonionic surfactant. The term “ionic surfactant” includes cationic, anionic, and zwitterionic surfactants. The term “cationic surfactant” refers to a surfactant with a cationic head group. The term “anionic surfactant” refers to a surfactant with an anionic head group.

As used herein, the term “pharmaceutically acceptable” or “pharmacologically acceptable”, when used herein, refers to a composition that is generally safe, non-toxic, and neither biologically nor otherwise undesirable and does not substantially produce adverse allergic or immunological reactions, when administered to a host (e.g., an animal or a human). Such a formulation includes any pharmaceutically acceptable dosage form.

As used herein, the term “pharmaceutically acceptable salt” or “salt thereof” means a salt which is pharmaceutically acceptable, as defined above, and which has a desired pharmacological activity. Such salts include acid addition salts formed with organic and inorganic acids, such as hydrogen chloride, hydrogen bromide, hydrogen iodide, sulfuric acid, phosphoric acid, acetic acid, glycolic acid, maleic acid, malonic acid, oxalic acid, methanesulfonic acid, trifluoroacetic acid, fumaric acid, succinic acid, tartaric acid, citric acid, benzoic acid, and ascorbic acid. Base addition salts are formed with organic and inorganic bases, such as sodium, ammonia, potassium, calcium, ethanolamine, diethanolamine, N-methylglucamine, and choline. Included are all pharmaceutically acceptable salts or compounds represented by the formulas used herein.

The term “pharmaceutically acceptable salt”, when used herein, refers to a pharmaceutically acceptable organic or inorganic acid or base salt of the compound, depending on the structure thereof. Representative examples of such a pharmaceutically acceptable salt include alkali metal salts, alkaline-earth salts, ammonium salts, and water-soluble and water-insoluble salts, such as acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulanate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methylene-bis-2-hydroxy-3-naphthoate, embonate), pantothenate, phosphate/diphosphate, picrate, polygalacturonate, propionate, p-toluenesulfonate, salicylate, stearate, subacetate, succinate, sulfate, subsalicylate, tannate, tartrate, teoclate, tosylate, triethiodide, and valerate.

As used herein, the term “hydrate” means a form of a compound, wherein water molecules are combined at a specific ratio as an essential part of the structure complex of the compound.

As used herein, the term “solvate” means a form of a compound, wherein solvent molecules are combined at a specific ratio as an essential part of the structure complex of the compound.

As used herein, the term “prodrug” means a compound that is metabolized or otherwise converted to an active or more active form with respect to at least one property, after administration. To produce a prodrug, a pharmaceutically active compound is modified chemically to render it less active or inactive, but the chemical modification is such that an active form of the compound is generated by metabolic or other biological processes. A prodrug can have, relative to the drug, altered metabolic stability or transport characteristics, fewer side effects or lower toxicity (Nogrady, 1985, Medicinal Chemistry A Biochemical Approach, Oxford University Press, New York, pages 388-392). Prodrugs can also be prepared using compounds that are not drugs.

The term “ophthalmically acceptable” with respect to a formulation, composition, or ingredient of the present disclosure indicates that the formulation, composition, or ingredient has no persistent harmful effect on the treated eye or the functioning thereof, or on the general health of the subject being treated, exhibiting transient effects such as minor irritation or a “stinging” sensation.

The term “active agent” or “active ingredient” is used herein to refer to a chemical material or compound that induces a desired beneficial effect when administered to a patient. Also included are salts, derivatives and analogs of those compounds or classes of compounds specifically mentioned (e.g., rifamycin compounds) that also induce the desired effect. For example, the term “rifampicin” when used herein includes pharmaceutically acceptable salts thereof and derivatives thereof.

The terms “buffer solution” or “buffer agent” refer to materials, which, when added to a solution, cause the solution to resist changes in pH values.

The term “dilution” refers to dilution of the formulation of the present invention or those derived from the formulation of the present invention using, for example, an aqueous system comprised of physiologically balanced saline solution (PBS), such as phosphate buffered saline, or water, or other water soluble components, to the desired final concentration.

As used herein, the terms “final concentration” and “final concentration in the composition” refer to the concentration of an indicated component in a complete composition, formulation, pharmaceutical composition, or any other medium thereof. Final concentration is relative to the final amount of complete composition, formulation, pharmaceutical composition, or any other medium thereof, including, but not limited to, e.g., pharmaceutically acceptable sterile isotonic aqueous or nonaqueous solution, dispersion, suspension, emulsion, or sterile powder for use in the methods and compositions provided herein. Final concentration can be indicated in any concentration unit(s) known in the art including, but not limited to, e.g., % by weight or mass, % w/w, % w/v, M, g/100 mL of the composition, etc.

As used herein, the term “increase” or “enhance” means to alter positively by at least about 5%, including, but not limited to, alter positively by about 5%, by about 10%, by about 25%, by about 30%, by about 50%, by about 75%, or by about 100%.

As used herein, the term “ligand” refers to a molecule that binds to a receptor. In particular, the ligand binds a receptor on another cell, allowing for cell-to-cell recognition and/or interaction.

The terms “polypeptide”, “peptide”, and “protein” are used interchangeably herein to refer to a polymer of amino acid residues. The terms apply to naturally occurring amino acid polymers as well as amino acid polymers in which one or more amino acid residues is a non-naturally occurring amino acid, e.g., an amino acid analog. The terms encompass amino acid chains of any length, including full length proteins, wherein the amino acid residues are linked by covalent peptide bonds.

As used herein, the term “reduce” means to alter negatively by at least about 5%, including, but not limited to, alter negatively by about 5%, by about 10%, by about 25%, by about 30%, by about 50%, by about 75%, or by about 100%.

As used herein, the terms “subject”, “individual”, or “patient” are used interchangeably and refer to an individual organism, a vertebrate, or a mammal and includes humans, non-human primates, rodents, livestock animal species, wild animals, and the like (e.g., which is to be the recipient of a particular treatment, or from whom cells are harvested). In certain embodiments, the individual, patient or subject is a human.

As used herein, the term “administration” of an agent to a subject includes any route of introducing or delivering the agent to a subject to perform its intended function. Administration can be carried out by any suitable route, including, but not limited to, ocular, topical, intravenously, intramuscularly, intraperitoneally, subcutaneously, and other suitable routes as described herein. The methods and compositions of the present disclosure preferably include intraocular administration, ophthalmic administration, or topical administration to the eye. As used herein intraocular administration and ophthalmic administration both refer to introducing or delivering the agent to a subject carried out by introduction or delivery of said agent to the eye. Administration includes self-administration and the administration by another.

The terms “treat”, “treating” or “treatment”, as used herein, include alleviating, abating or ameliorating a disease or condition, or one or more symptoms thereof, preventing additional symptoms, ameliorating or preventing the underlying metabolic causes of symptoms, inhibiting the disease or condition, for example, arresting or suppressing the development of the disease or condition, relieving the disease or condition, causing regression of the disease or condition, relieving a condition caused by the disease or condition, or suppressing the symptoms of the disease or condition, and are also intended to include prophylaxis. The terms also include relieving the disease or conditions, for example, causing the regression of clinical symptoms. The terms further include achieving a therapeutic benefit and/or a prophylactic benefit. Such a therapeutic benefit is intended to mean eradication or amelioration of the underlying disorder being treated. Also, such a therapeutic benefit is achieved with the eradication or amelioration of one or more of the physiological symptoms associated with the underlying disorder such that an improvement is observed in the individual, notwithstanding that the individual is still be afflicted with the underlying disorder. For prophylactic benefit, the composition is administered to an individual at risk of developing a particular disease, or to an individual reporting one or more of the physiological symptoms of a disease, even though the diagnosis of this disease has not yet been made. “Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. Therapeutic effects of treatment include, without limitation, inhibiting recurrence of disease, alleviation of symptoms, diminishment of any direct or indirect pathological consequences of the disease, preventing metastases, decreasing the rate of disease progression, amelioration or palliation of the disease state, and remission or improved prognosis. It is also to be appreciated that the various modes of treatment of diseases as described herein are intended to mean “substantial”, which includes total but also less than total treatment, and wherein some biologically or medically relevant result is achieved. In certain aspects, the treatment is a continuous prolonged treatment for a chronic disease or a single, or few time administrations for the treatment of an acute condition.

The terms “preventing” or “prevention” refer to a reduction in risk of acquiring a disease or disorder (i.e., causing at least one of the clinical symptoms of the disease not to develop in a subject that is exposed to or predisposed to the disease but does not yet experience or display symptoms of the disease). The terms further include causing the clinical symptoms not to develop, for example, in a subject at risk of suffering from such a disease or disorder, thereby substantially averting onset of the disease or disorder.

The term “macular degeneration” refers to a variety of degenerative conditions characterized by central visual loss due to deterioration of the macula. One of these conditions is age related macular degeneration (AMD), which exists in both “dry” and “wet” forms.

The term “ocular neovascularization” refers to the abnormal development, proliferation, and/or growth of blood vessels on or in the eye, for example, on the retinal surface.

### Ophthalmic Compositions

### Rifamycin Compounds

In certain aspects, the disclosure provides an ophthalmic composition comprising, or consisting essentially of, or yet further consisting of an effective amount of one or more rifamycin compounds or a pharmaceutically acceptable salt thereof. The rifamycin class of antibiotic was originally isolated from cultures of Streptomyces mediterranei. Due to the large number of available analogues and derivatives generated synthetically, rifamycin has been widely utilized in the elimination of pathogenic bacteria that have become resistant to commonly used antibiotics. Rifamycin is effective against mycobacteria, and is therefore used to treat chronic infections including tuberculosis (TB), leprosy, and Mycobacterium avium complex (MAC) infections. Along with its pulmonary effects, TB is also known to affect other organs including ocular tissues. Therefore, the effects of rifamycin on ocular TB and other ocular disorders has been a targeted research area of interest in the field. Rifampicin has been shown to exhibit strong neovascularization effects, whereby major neovascularization genes such as VEGF, HGF etc. are strongly inhibited.

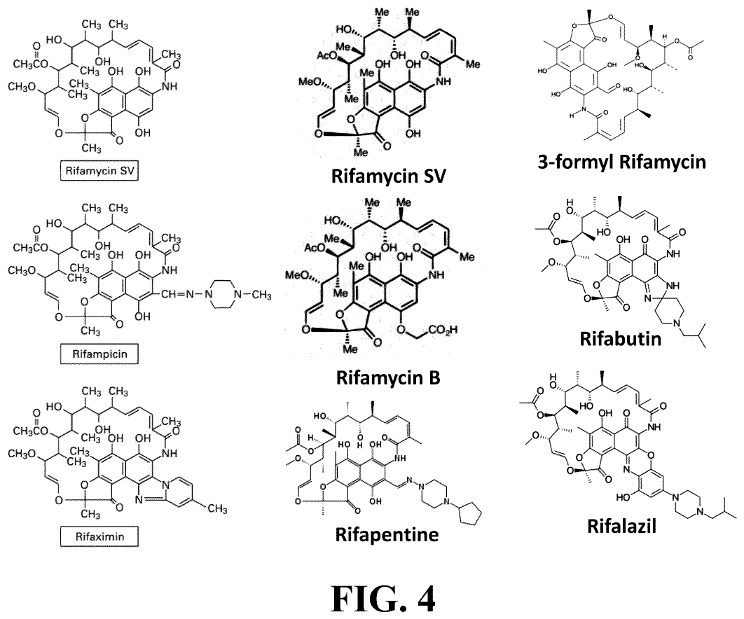

Examples of suitable rifamycin compounds of the present disclosure, include rifampicin (rifampin), rifabutin, rifapentine, rifalazil, rifaximin, rifamycin SV, 3-formyl rifamycin, rifamycin B, or a pharmaceutically acceptable salt or derivative thereof. Suitable rifamycin compounds of the present disclosure are commercially available from, for example, Sigma-Aldrich, or can be prepared by known procedures. Chemical structures of exemplary suitable rifamycin compounds of the present disclosure are shown in FIG. 4. Synthesis of simple rifamycin derivatives is well known in the present technical field. For example, the synthesis of rifampin (U.S. Pat. No. 3,342,810), rifabutin (U.S. Pat. No. 4,219,478), and rifalazil (U.S. Pat. No. 4,983,602) are known in the present technical field and incorporated herein by reference.

In one aspect, the disclosure provides an ophthalmic composition comprising, consisting essentially of, or yet further consisting of an effective amount of one or more rifamycin compounds or a pharmaceutically acceptable salt thereof. In some embodiments, the one or more rifamycin compounds is selected from the group consisting of rifamycin SV, 3-formyl rifamycin SV, rifampicin, rifabutin, rifapentine, and rifaximin. In some embodiments, the one or more rifamycin compounds is rifamycin SV. In some embodiments, the one or more rifamycin compounds is 3-formyl rifamycin SV. In some embodiments, the one or more rifamycin compounds is rifampicin. In some embodiments, the one or more rifamycin compounds is rifabutin. In some embodiments, the one or more rifamycin compounds is rifapentine. In some embodiments, the one or more rifamycin compounds is rifaximin.

In some embodiments, the concentration of one or more rifamycin compounds is appropriately determined in the range of about 0.00001% w/w to about 50% w/w. As used herein, “% w/w” indicates the proportion or weight concentration of a substance within a mixture, as measured by weight or mass. In some embodiments, the effective amount of the one or more rifamycin compounds comprises a final concentration in the composition of at least about 0.05% w/w, at least about 0.1% w/w, at least about 2% w/w, at least about 0.25% w/w, at least about 0.3% w/w, at least about 0.35% w/w, at least about 0.4% w/w, at least about 0.45% w/w, at least about 0.5% w/w, at least about 0.55% w/w, at least about 0.6% w/w, at least about 0.65% w/w, at least about 0.7% w/w, at least about 0.75% w/w, at least about 0.8% w/w, at least about 0.85% w/w, at least about 0.9% w/w, at least about 0.95% w/w, at least about 1% w/w, at least about 1.05% w/w, at least about 1.1% w/w, at least about 1.15% w/w, at least about 1.2% w/w, at least about 1.25% w/w, at least about 1.3% w/w, at least about 1.35% w/w, at least about 1.4% w/w, at least about 1.45% w/w, or at least about 1.5% w/w. In some embodiments, the effective amount of the one or more rifamycin compounds comprises a final concentration in the composition of at least 0.25 to at least 1.5% w/w, or at least 0.5 to at least 1.5% w/w, or at least 0.75 to at least 1.5% w/w, or at least 1 to at least 1.5% w/w, or at least 1.5 to at least 3% w/w, or least 3 to at least 5% w/w, or at least 5% w/w.

In some embodiments the effective amount of the one or more rifamycin compounds comprises a final concentration in the composition of about 0.00001% w/w to 0.0001% w/w, about 0.0001% w/w to about 0.0005% w/w, about 0.0005% w/w to about 0.001% w/w, about 0.001% w/w to about 0.005% w/w, about 0.005% w/w to about 0.01% w/w, about 0.01% w/w to about 50% w/w, about 0.01% w/w to about 0.05% w/w, about 0.05% w/w to about 0.1% w/w, about 0.05% w/w to about 40% w/w, about 0.1% w/w to about 0.25% w/w, about 0.1% w/w to about 30% w/w, about 0.25% w/w to about 0.5% w/w, about 0.5% w/w to about 20% w/w, about 0.5% w/w to about 1.0% w/w, about 1.0% w/w to about 2.0% w/w, about 2.0% w/w to about 5.0% w/w, about 1.0% w/w to about 10% w/w, about 1.5% w/w to about 5% w/w, about 2.0% w/w to about 3.0% w/w, about 5.0% w/w to about 10.0% w/w, and a range between any given two values from these values, or a value lower than any given value from these values. In some embodiments, the effective amount of the one or more rifamycin compounds comprises a final concentration in the composition of about 50% w/w, 30% w/w, 20% w/w, 10% w/w, about 8% w/w, about 7% w/w, about 5% w/w, about 4% w/w, about 3.5% w/w, about 3% w/w, about 2.5% w/w, about 2% w/w, about 1.5% w/w, about 1% w/w, about 0.5% w/w, about 0.25% w/w, about 0.1% w/w, about 0.05% w/w, about 0.01% w/w, about 0.005% w/w, about 0.002% w/w, about 0.001% w/w, about 0.0005% w/w, about 0.0001% w/w, or about 0.00001%.

In some embodiments, the lower limit of the effective amount of the one or more rifamycin compounds comprises a final concentration in the composition selected from the range of about 0.00001% w/w to about 50% w/w, and for example, can be set to be about 0.00001% w/w, 0.0001% w/w, 0.0005% w/w, 0.001% w/w, or 0.005% w/w (but is not limited to such % w/w). In some embodiments, the upper limit the effective amount of the one or more rifamycin compounds comprises a final concentration in the composition selected from the above-described range, and for example, can be about 50% w/w, 40% w/w, 30% w/w, or 20% w/w.

In some embodiments, the concentration of one or more rifamycin compounds can be appropriately determined in the range of about 0.0001% by weight to about 50% by weight. As used herein, “% by weight” can be indicated as “g/100 mL of the composition” or “weight/volume (w/v).”

In some embodiments, the lower limit of the effective amount of the one or more rifamycin compounds comprises a final concentration in the composition selected from the range of about 0.00001% by weight to about 50% by weight, and for example, can be set to be about 0.00001% by weight, 0.0001% by weight, 0.0005% by weight, 0.001% by weight, or 0.005% by weight (but is not limited to such % by weight). In some embodiments, the upper limit the effective amount of the one or more rifamycin compounds comprises a final concentration in the composition selected from the above-described range, and for example, can be about 50% by weight, 40% by weight, 30% by weight, or 20% by weight.

In some embodiments the effective amount of the one or more rifamycin compounds comprises a final concentration in the composition of about 0.00001% by weight to about 0.0001% by weight, about 0.0001% by weight to about 0.0005% by weight, about 0.0005% by weight to about 0.001% by weight, about 0.001% by weight to about 0.005% by weight, about 0.005% by weight to about 0.01% by weight, about 0.01% by weight to about 50% by weight, about 0.01% by weight to about 0.05% by weight, about 0.05% by weight to about 0.1% by weight, about 0.05% by weight to about 40% by weight, about 0.1% by weight to about 0.25% by weight, about 0.1% by weight to about 30% by weight, about 0.25% by weight to about 0.5% by weight, about 0.5% by weight to about 20% by weight, about 0.5% by weight to about 1.0% by weight, about 1.0% by weight to about 2.0% by weight, about 2.0% by weight to about 5.0% by weight, about 1.0% by weight to about 10% by weight, about 1.5% by weight to about 5% by weight, about 2.0% by weight to about 3.0% by weight, about 5.0% by weight to about 10.0% by weight, and a range between any given two values from these values, or a value lower than any given value from these values. In some embodiments, the effective amount of the one or more rifamycin compounds comprises a final concentration in the composition of about 50% by weight, 30% by weight, 20% by weight, 10% by weight, about 8% by weight, about 7% by weight, about 5% by weight, about 4% by weight, about 3.5% by weight, about 3% by weight, about 2.5% by weight, about 2% by weight, about 1.5% by weight, about 1% by weight, about 0.5% by weight, about 0.25% by weight, about 0.1% by weight, about 0.05% by weight, about 0.01% by weight, about 0.005% by weight, about 0.002% by weight, about 0.001% by weight, about 0.0005% by weight, about 0.0001% by weight, or about 0.00001%.

### Buffer Agents

In one aspect, the ophthalmic compositions of the present disclosure includes one or more buffer agents. A buffering agent can be a weak acid or weak base present in the composition in order to maintain the pH of the composition. In some embodiments, the ophthalmic compositions of the present disclosure comprises, consists essentially of, or yet further consists of one or more buffer agents for maintaining the pH of the composition. In some embodiments, the pH of the ophthalmic compositions of the present disclosure is, for example, a pH value of about 1 to 12, a pH value of about 2 to 12, a pH value of about 3 to 9, or a pH value of about 3 to 7.5. The pH value of the ophthalmic compositions of the present disclosure can be adjusted up to the first, second, or third decimal place. For example, when the pH is adjusted to the third decimal place, the lower limit value is, for example, 1.000, 2.000, 3.000, 4.000, 5.000, etc., whereas the upper limit value is, for example, 12.000, 11.000, 10.000, 9.000, 8.000, etc.

In some embodiments, the ophthalmic compositions of the present disclosure further comprises, or consists essentially of, or yet further consists of, one or more buffer agents. In some embodiments, the one or more buffer agents is selected from the group consisting of acetic acid, boric acid, citric acid, lactic acid, phosphoric acid, hydrochloric acid-potassium chloride, glycine, aconitic acid, citric acid-phosphoric acid, succinic acid, phthalic acid, maleic acid, cacodylic acid, tris (trishydroxymethylaminomethane), barbituric acid, borax, 2-amino-2-methyl-1,3-propanediol (Ammediol), sodium carbonate-sodium bicarbonate, HEPES (4-(2-hydroxyethyl)-1-piperazineethanesulfonic acid), ACES (N-(2-acetamido)-2-aminoethanesulfonic acid), ADA (N-(2-acetamido) iminodiacetic acid), BES (N,N-bis(2-hydroxyethyl)-2-aminoethanesulfonic acid), Bicine (N,N-bis(2-hydroxyethyl) glycine), Bis-Tris (bis(2-hydroxyethyl) iminotris(hydroxymethyl) methane), CAPS (N-cyclohexyl-3-aminopropanesulfonic acid), CAPSO (N-cyclohexyl-2-hydroxy-3-aminopropanesulfonic acid), CHES (N-cyclohexyl-2-aminoethanesulfonic acid), DIPSO (3-[N,N-bis(2-hydroxyethyl)amino]-2-hydroxypropanesulfonic acid), EPPS (3-[4-(2-hydroxyethyl)-1-piperazinyl] propanesulfonic acid), HEPES-Na (sodium 2-[4-(2-hydroxyethyl)-1-piperazinyl] ethanesulfonate), HEPPSO (2-hydroxy-3-[4-(2-hydroxyethyl)-1-piperazinyl] propanesulfonic acid, monohydrate), MES (2-morpholinoethanesulfonic acid, monohydrate), MOPS (3-morpholinopropanesulfonic acid), MOPSO (2-hydroxy-3-morpholinopropanesulfonic acid), PIPES (piperazine-1,4-bis(2-ethanesulfonic acid)), POPSO (piperazine-1,4-bis(2-hydroxy-3-propanesulfonic acid), dihydrate), TAPSO (2-hydroxy-N-tris(hydroxymethyl)methyl-3-aminopropanesulfonic acid), TES (N-tris(hydroxymethyl)methyl-2-aminoethanesulfonic acid), Tricine (N-[tris(hydroxymethyl)methyl]glycine), and hydrochloric acid; bases, such as sodium hydroxide, sodium phosphate, sodium borate, sodium citrate, sodium acetate, and sodium lactate; and buffer agents, such as citrate/dextrose, sodium bicarbonate, and ammonium chloride, and citrate, phosphate, borate, bicarbonate, sodium salt and potassium, comprising a combination thereof.

In some embodiments, the ophthalmic compositions of the present disclosure comprises, consists essentially of, or yet further consists of one or more buffer agents. In some embodiments, the one or more buffer agents is Tris. Examples of a representative Tris buffer solution used in the present disclosure includes, but is not limited to, a Tris-HCl buffer solution, a TE buffer solution (for example, having a composition of 10 mM Tris/Tris-HCl and 1 mM EDTA), a TAE buffer solution (for example, having a composition of 40 mM Tris/Tris-acetate and 1 mM EDTA), a TBE buffer solution (for example, having a composition of 89 mM Tris/Tris-borate and 2 mM EDTA), and TBS (for example, having a composition of 10 mM Tris/Tris-HCl and 150 mM NaCl).

In some embodiments, the ophthalmic compositions of the present disclosure comprises, consists essentially of, or yet further consists of one or more buffer agents. In some embodiments, the buffer agent comprises a final concentration in the composition of about 1 mM to about 100 mM. In some embodiments, the buffer agent comprises a final concentration in the composition of about 10 mM to about 100 mM. In some embodiments, the buffer agent comprises a final concentration in the composition of about 20 mM to about 100 mM. In some embodiments, the buffer agent comprises a final concentration in the composition of about 30 mM to about 100 mM. In some embodiments, the buffer agent comprises a final concentration in the composition of about 40 mM to about 100 mM. In some embodiments, the buffer agent comprises a final concentration in the composition of about 50 mM to about 100 mM. In some embodiments, the buffer agent comprises a final concentration in the composition of about 60 mM to about 100 mM. In some embodiments, the buffer agent comprises a final concentration in the composition of about 70 mM to about 100 mM. In some embodiments, the buffer agent comprises a final concentration in the composition of about 80 mM to about 100 mM. In some embodiments, the buffer agent comprises a final concentration in the composition of about 90 mM to about 100 mM. In some embodiments, In some embodiments, the buffer agent comprises a final concentration in the composition of about 1 mM, about 2 mM, about 3 mM, about 4 mM, about 5 mM, about 6 mM, about 7 mM, about 8 mM, about 9 mM, about 10 mM, about 15 mM, about 20 mM, about 25 mM, about 30 mM, about 40 mM, about 50 mM, about 60 mM, about 70 mM, about 80 mM, about 90 mM, or about 100 mM.

### Pharmaceutically Acceptable Carrier

In some embodiments, the ophthalmic compositions of the present disclosure comprises, consists essentially of, or yet further consists of a pharmaceutically acceptable carrier. The pharmaceutically acceptable carrier for the present compositions can include, but are not limited to, amino acids, peptides, biological polymers, non-biological polymers, simple sugars or starches, inorganic salts, and gums, which can be present singly or in combinations thereof. The peptides used in the acceptable carrier can include, e.g., gelatin and/or albumin. Cellulose or its derivatives can be used in the pharmaceutically acceptable carrier. The sugar used in the acceptable carrier can be lactose and/or glucose. Other useful sugars which can be utilized in the pharmaceutical compositions include but are not limited to, fructose, galactose, lactitol, maltitol, maltose, mannitol, melezitose, myoinositol, palatinate, raffinose, stachyose, sucrose, trehalose, xylitol, hydrates thereof, and combinations of thereof. Binders can be included in the pharmaceutically acceptable carrier. Examples of binders include, but are not limited to, starches (for example, corn starch or potato starch), gelatin; natural or synthetic gums such as acacia, sodium alginate, powdered tragacanth, guar gum, cellulose or cellulose derivatives (for example, methylcellulose, ethyl cellulose, cellulose acetate); microcrystalline cellulose, polyvinyl pyrrolidone, and mixtures thereof. Inorganic salts used in the acceptable carrier can be a magnesium salt, for example, magnesium chloride or magnesium sulfate. Other inorganic salts can be used, for example, calcium salts. Examples of calcium salts include, but are not limited to, calcium chloride, calcium sulfate. Other examples of substances which can be used in the pharmaceutically acceptable carrier include, but are not limited to, vegetable oils, such as peanut oil, cottonseed oil, olive oil, corn oil; polyols such as glycerin, propylene glycol, polyethylene glycol; pyrogen-free water, isotonic saline, phosphate buffer solutions; emulsifiers, such as the Tweens®; wetting agents, lubricants, coloring agents, flavoring agents, preservatives.

In some embodiments, the pharmaceutically acceptable carrier comprises more than 90%, more than 80%, more than 70%, more than 60%, more than 50%, more than 40%, more than 30%, more than 20%, more than 10%, more than 9%, more than 8%, more than 6%, more than 5%, more than 4%, more than 3%, more than 2%, more than 1%, more than 0.5%, more than 0.4%, more than 0.3%, more than 0.2%, more than 0.1%, more than 0.09%, more than 0.08%, more than 0.07%, more than 0.06%, more than 0.05%, more than 0.04%, more than 0.03%, more than 0.02%, more than 0.01%, more than 0.009%, more than 0.008%, more than 0.007%, more than 0.006%, more than 0.005%, more than 0.004%, more than 0.003%, more than 0.002%, more than 0.001%, more than 0.0009%, more than 0.0008%, more than 0.0007%, more than 0.0006%, more than 0.0005%, more than 0.0004%, more than 0.0003%, more than 0.0002%, or more than 0.0001% of the pharmaceutical composition by w/w, w/v or v/v.

### Surfactants

In some embodiments, the ophthalmic compositions of the present disclosure comprises, consists essentially of, or yet further consists of one or more surfactants. Surfactant which can be used to form pharmaceutical compositions and dosage forms of the application include, but are not limited to, hydrophilic surfactants, lipophilic surfactants, and mixtures thereof. That is, a mixture of hydrophilic surfactants can be employed, a mixture of lipophilic surfactants can be employed, or a mixture of at least one hydrophilic surfactant and at least one lipophilic surfactant can be employed.

Suitable surfactants are known in the present technical field, and examples of the surfactant can include, but are not limited to, sorbitan ether esters of oleic acid (e.g., polysorbate 80 or Tween 20 and 80), polyoxyethylene castor oil, polyoxyethylene hydrogenated castor oil, cremophor, sodium alkylbenzene sulfonate, glycerol, lecithin, sucrose ester, polyoxyethylene-alkyl ether, polyoxyl stearate, polyoxyl 40 stearate, ethylene glycol monostearate, polyethylene glycol monostearate, polymers of oxyethylated octyl phenol (tyloxapol), propylene glycol, benzyl alcohol, macrogol, cyclodextrin, dibutylhydroxytoluene, sorbitol, trometamol, propylene glycol, mannitol, and polyoxyethylene polyoxypropylene glycol (e.g. polyoxyethylene (160) polyoxypropylene (30) glycol, or polyoxyethylene (200) polyoxypropylene (70) glycol), or combinations thereof. In some embodiments, the present ophthalmic composition comprises, consists essentially of, or yet further consists of polysorbate 80, polyoxyethylene hydrogenated castor oil, lecithin, or a combination thereof.

A suitable hydrophilic surfactant can generally have an HLB value of at least 10, while suitable lipophilic surfactants can generally have an HLB value of or less than about 10. A useful parameter that can be used to characterize the relative hydrophilicity and hydrophobicity of non-ionic amphiphilic compounds is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are generally considered to be compounds having an HLB value equal to or less than about 10. However, HLB value of a surfactant merely provides a rough guide generally used to enable formulation of industrial, pharmaceutical and cosmetic emulsions.

Hydrophilic surfactants can be either ionic or non-ionic. Suitable ionic surfactants include, but are not limited to, alkylammonium salts, fatty acid derivatives of amino acids, glyceride derivatives of amino acids, fusidic acid salts, oligopeptides, and polypeptides, oligopeptides, and polypeptides, lecithins and hydrogenated lecithins, lysolecithins and hydrogenated lysolecithins, phospholipids and derivatives thereof, fatty acid salts, lysophospholipids and derivatives thereof, carnitine fatty acid ester salts, salts of alkylsulfates, sodium docusate, acylactylates, mono- and di-acetylated tartaric acid esters of mono- and di-glycerides, succinylated mono- and di-glycerides, citric acid esters of mono- and di-glycerides, and mixtures thereof.

Within the aforementioned group, ionic surfactants include, but are not limited to, lecithins, lysolecithin, phospholipids, lysophospholipids and derivatives thereof, carnitine fatty acid ester salts, fatty acid salts, salts of alkylsulfates, sodium docusate, acylactylates, mono- and di-acetylated tartaric acid esters of mono- and di-glycerides, succinylated mono- and di-glycerides, citric acid esters of mono- and di-glycerides, and mixtures thereof. Ionic surfactant can also include, but are not limited to, alkyl diaminoethyl glycine hydrochloride solution, benzododecinium bromide, benzalkonium chloride, benzethonium chloride, sodium polystyrene sulfonate, benzoic acid (benzoate), ethylenediaminetetraacetic acid, thimerosal, sodium thiosulfate, citric acid (citrate), glutamic acid (glutamate), sorbic acid, sodium dehydroacetate, and acetate.

Ionic surfactants can be the ionized forms of lactylic esters of fatty acids, lecithin, lysolecithin, phosphatidylethanolamine, phosphatidylcholine, phosphatidylglycerol, phosphatidic acid, phosphatidylserine, lysophosphatidylcholine, lysophosphatidylserine, lysophosphatidylethanolamine, lysophosphatidylglycerol, lysophosphatidic acid, PEG-phosphatidylethanolamine, PVP-phosphatidylethanolamine, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, linoleate, linolenate, stearate, ricinoleate, lauryl sulfate, teracecyl sulfate, docusate, lauroyl carnitines, palmitoyl carnitines, myristoyl carnitines, and salts and mixtures thereof.

Hydrophilic non-ionic surfactants can include, but not limited to, alkylglucosides, alkylthioglucosides, alkylmaltosides, lauryl macrogolglycerides, polyoxyalkylene alkyl ethers such as polyethylene glycol alkyl ethers, polyoxyalkylene alkylphenols such as polyethylene glycol alkyl phenols, polyethylene glycol glycerol fatty acid esters, polyoxyalkylene alkyl phenol fatty acid esters such as polyethylene glycol fatty acids monoesters and polyethylene glycol fatty acids diesters, polyglycerol fatty acid esters, polyoxyethylene-polyoxypropylene block copolymers and mixtures thereof, polyoxyalkylene sorbitan fatty acid esters such as polyethylene glycol sorbitan fatty acid esters, hydrophilic transesterification products of a polyol with at least one member of the group consisting of glycerides, vegetable oils, hydrogenated vegetable oils, fatty acids, and sterols, polyoxyethylene sterols and derivatives or analogues thereof, polyoxyethylated vitamins and derivatives thereof, polyethylene glycol sorbitan fatty acid esters and hydrophilic transesterification products of a polyol with at least one member of the group consisting of triglycerides, vegetable oils, and hydrogenated vegetable oils. The polyol can be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-12 oleate, PEG-15 oleate, PEG-20 oleate, PEG-20 laurate, PEG-32 dilaurate, PEG-32 laurate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-20 trioleate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-50 hydrogenated castor oil, PEG-40 castor oil, PEG-35 castor oil, PEG-60 castor oil, PEG-40 palm kernel oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phytosterol, PEG-30 soya sterol, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, but are not limited to, fatty alcohols, glycerol fatty acid esters, acetylated glycerol fatty acid esters, lower alcohol fatty acids esters, propylene glycol fatty acid esters, sorbitan fatty acid esters, polyethylene glycol sorbitan fatty acid esters, sterols and sterol derivatives, polyoxyethylated sterols and sterol derivatives, polyethylene glycol alkyl ethers, sugar ethers, sugar esters, hydrophobic transesterification products of a polyol with at least one member of the group consisting of glycerides, vegetable oils, hydrogenated vegetable oils, fatty acids and sterols, oil-soluble vitamins/vitamin derivatives, lactic acid derivatives of mono- and di-glycerides, and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In some preferred embodiments of the present disclosure, the ophthalmic composition comprises, consists essentially of, or yet further consists of a surfactant selected from polysorbate 80, Tween 80, Tween 20, polyoxyethylene hydrogenated castor oil, polyoxyethylene alkyl ether, and lecithin, or a combination thereof.

In some embodiments, the ophthalmic compositions of the present disclosure do not comprise an ionic surfactant at all. In some embodiments, the ophthalmic compositions of the present disclosure comprise, consist essentially of, or yet further consist of an ionic surfactant with a final concentration in the composition of at least about 0.00001% by weight to at least about 50% by weight. In some embodiments, the ionic surfactant comprises a final concentration in the composition of about 0.00005% by weight or less. In some embodiments, the ionic surfactant comprises a final concentration in the composition of at least about 0.00005% by weight.

In some embodiments, the ophthalmic compositions of the present disclosure comprise a surfactant with a final concentration in the composition with a lower concentration limit of about 0.00001% by weight, about 0.0001% by weight, or about 0.001% by weight. In some embodiments, the ophthalmic compositions of the present disclosure comprise a surfactant with a final concentration in the composition with an upper limit of about 50% by weight, about 40% by weight, about 30% by weight, about 20% by weight, or about 10% by weight (but are not limited to such % by weight). The appropriate range of surfactant concentration is set from these upper and lower limit values. For example, the amount of a surfactant in the present composition can be about 0.00001% by weight to about 50% by weight, about 0.0001% by weight to about 50% by weight, about 0.001% by weight to about 50% by weight, about 0.01% to about 50% by weight, about 0.1% to about 50% by weight, about 0.1% to about 40% by weight, about 0.1% to about 30% by weight, about 1% to about 20% by weight, or about 2% to about 10% by weight.

In some embodiments, the ophthalmic compositions of the present disclosure comprise, consist essentially of, or yet further consist of a surfactant with a final concentration in the composition of about 0.00001% by weight to about 50% by weight, about 0.00005% by weight to about 50% by weight, about 0.0001% by weight to about 50% by weight, about 0.001% by weight to about 50% by weight, about 0.01% by weight to 20% by weight, about 0.01% by weight to 15% by weight, about 0.15% by weight to 10% by weight, about 0.2% by weight to 5% by weight, about 0.25% by weight to 3% by weight, about 0.3% by weight to 2% by weight, about 0.1% by weight to 20% by weight, about 1% by weight to 10% by weight, about 2% by weight to 10% by weight, about 2% by weight to 8% by weight, about 2% by weight to 5% by weight, about 5% by weight to 10% by weight, about 5% by weight to 20% by weight, and a range between any given two values from these values, or a value lower than any given value from these values.

In the present disclosure, the final concentration of the surfactant in the composition can be adjusted in the range of up to the first, second, third, fourth, or fifth decimal place. For example, the concentration of the surfactant can be set, as appropriate, in the range of 0.00001% to 50.00% by weight, 0.0001% to 50.00% by weight, 0.001% to 50.00% by weight, 0.01% to 50.00% by weight, or 0.10% to 50.00% by weight.

Lubricants that can be used in the ophthalmic compositions disclosed herein, and include, but are not limited to, agar, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laurate, or mixtures thereof. Additional lubricants include, by way of example, a syloid silica gel, a coagulated aerosol of synthetic silica, or mixtures thereof. A lubricant is optionally added, in an amount of less than about 1 weight percent of the pharmaceutical composition.

The composition can include one or more pharmaceutically acceptable additives, which include, but are not limited to, detackifiers, anti-foaming agents, buffering agents, antioxidants, polymers, preservatives, chelating agents, odorants, opacifiers, suspending agents, fillers, plasticizers, and mixtures thereof.

### Viscosity Imparting Agents

In some embodiments of the present disclosure, the ophthalmic composition further comprises, consists essentially of, or yet further consists of one or more viscosity imparting agents. Viscosity imparting agents can function to increase the viscosity of an ophthalmic composition of the present disclosure, resulting in increased ocular contact times, increased mucosa adhesion, and decreased drainage. In some embodiments, the viscosity imparting agent increases the viscosity of the composition or an ophthalmic solution, an ointment, or a suspension. In some embodiments, the viscosity imparting agent increases an ocular contact time, thereby decreasing the drainage rate. In some embodiments, the viscosity imparting agent increases mucosa adhesion and ocular bioavailability, and/or imparts lubricating effects. Examples of suitable viscosity imparting agents includes, but are not limited to, petrolatum, liquid paraffin, light liquid paraffin, carboxyvinyl polymers (e.g., Carbopol 934P or 974P), cellulosic polymers (e.g., carboxymethyl cellulose, hydroxypropylmethyl cellulose, hydroxyethyl cellulose or the like), polysaccharides (e.g., xanthan gum), polyvinyl pyrrolidone, polyvinyl alcohol, chondroitin sulfate, lanolin, hyaluronic acid, propylene glycol, and a combination thereof.

In some preferred embodiments of the present disclosure, the one or more viscosity imparting agents comprises, consists essentially of, or yet further consists of petrolatum or liquid paraffin. In a preferred embodiment of the present disclosure, the one or more viscosity imparting agents comprises, consists essentially of, or yet further consists of petrolatum. In another preferred embodiment of the present disclosure, the one or more viscosity imparting agents comprises, consists essentially of, or yet further consists of liquid paraffin.

In some embodiments, the ophthalmic composition comprises, consists essentially of, or yet further consists of a viscosity imparting agent with a final concentration in the composition of about 0.00001% by weight to about 50% by weight, about 0.00005% by weight to about 50% by weight, about 0.0001% by weight to about 50% by weight, about 0.001% by weight to about 50% by weight, about 0.01% by weight to 20% by weight, about 0.1% by weight to 15% by weight, about 0.15% by weight to 10% by weight, about 0.2% by weight to 5% by weight, about 0.25% by weight to 3% by weight, about 0.3% by weight to 2% by weight, about 0.5% by weight to 2% by weight, about 0.1% by weight to 20% by weight, about 1% by weight to 10% by weight, about 2% by weight to 10% by weight, about 2% by weight to 8% by weight, about 2% by weight to 5% by weight, about 5% by weight to 10% by weight, about 5% by weight to 20% by weight, 10% by weight to 40% by weight, 20% by weight to 50% by weight, 30% by weight to 60% by weight, 40% by weight to 70% by weight, 50% by weight to 80% by weight, 60% by weight to 90% by weight, 70% by weight to 100% by weight, 80% by weight to 100% by weight, 90% by weight to 100% by weight, about 100%, about 90%, about 80%, about 70%, about 60%, about 50%, about 40%, about 30%, about 20%, about 10%, about 5% and a range between any given two values from these values, or a value lower than any given value from these values. In some embodiments, the ophthalmic composition comprises, consists essentially of, or yet further consists of a viscosity imparting agent with a final concentration in the composition about 0.01% by weight to 100% by weight.

The viscosity of a solution can be measured in units of Pa·s (Pascal second; hereinafter—“PaS”) or mPa·s (millipascal second; hereinafter—“mPaS”). Viscosity can also be measured in cP (centipoise), where 0.001 PaS is equivalent to 1 mPaS, which is further equivalent to 1 cP. The viscosity of a solution depends strongly on temperature, and typically decreases with increasing temperature. Viscosity can be measured using a rheometer or a viscometer. Viscosity can be measured using a rheometer or viscometer comprising a temperature control unit, which maintains and controls a temperature at least in the range of 0.0±0.1° C., 5.0±0.1° C., 10.0±0.1° C., 15.0±0.1° C., 20.0±0.1° C., 25.0±0.1° C., 37.0±0.1° C., 50.0±0.1° C., 75.0±0.1° C., 95.0±0.1° C., 98.0±0.1° C., or 100±0.1° C.

Shear velocity can also be determined using a rheometer comprising a temperature control unit, which maintains and controls a temperature at least in the range of 0.0±0.1° C., 5.0±0.1° C., 10.0±0.1° C., 15.0±0.1° C., 20.0±0.1° C., 25.0±0.1° C., 37.0±0.1° C., 50.0±0.1° C., 75.0±0.1° C., 95.0±0.1° C., 98.0±0.1° C., or 100±0.1° C. Shear is the relative motion between adjacent layers of a fluid. Shear velocity is the rate of change of velocity at which a fluid layer passes over another adjacent fluid layer. In some embodiments, the ophthalmic compositions of the present disclosure comprise a shear velocity in the range of 0 s⁻¹ to 200 s⁻¹, or 5 s⁻¹ to 200 s⁻¹ or 10 s⁻¹ to 200 s⁻¹, or 20 s⁻¹ to 200 s⁻¹, or 30 s⁻¹ to 200 s⁻¹, or 40 s⁻¹ to 200 s⁻¹, or 50 s⁻¹ to 200 s⁻¹, or 75 s⁻¹ to 200 s⁻¹, or 100 s⁻¹ to 200 s⁻¹, or 125 s⁻¹ to 200 s⁻¹, or 150 s⁻¹ to 200 s⁻¹, or 175 s⁻¹ to 200 s⁻¹, 190 s⁻¹ to 200 s⁻¹, 0 s⁻¹ to 400 s⁻¹, or 205 s⁻¹ to 400 s⁻¹, or 210 s⁻¹ to 400 s⁻¹, or 220 s⁻¹ to 400 s⁻¹, or 230 s⁻¹ to 400 s⁻¹, or 240 s⁻¹ to 400 s⁻¹, or 250 s⁻¹ to 400 s⁻¹, or 275 s⁻¹ to 400 s⁻¹, or 300 s⁻¹ to 400 s⁻¹, or 325 s⁻¹ to 400 s⁻¹, or 350 s⁻¹ to 400 s⁻¹, or 375 s⁻¹ to 400 s⁻¹, or 380 s⁻¹ to 400 s⁻¹, or 0 s⁻¹ to 600 s⁻¹, or 0 s⁻¹ to 800 s⁻¹, or 0 s⁻¹ to 1,000 s⁻¹. In some further embodiments, the ophthalmic compositions of the present disclosure comprise a shear velocity measured at a temperature at least in the range of 0.0±0.1° C., or 5.0±0.1° C., or 10.0±0.1° C., or 15.0±0.1° C., or 20.0±0.1° C., or 25.0±0.1° C., or 37.0±0.1° C., or 50.0±0.1° C., or 75.0±0.1° C., or 95.0±0.1° C., or 98.0±0.1° C., or 100±0.1° C.

In some embodiments, the ophthalmic compositions of the present disclosure comprise a final viscosity in the range of 1 mPaS to 5 mPaS, or 5 mPaS to 25 mPaS, or 25 mPaS to 100 mPaS, or 100 mPaS to 200 mPaS, or 150 mPaS to 300 mPaS, or 200 mPaS to 500 mPaS, or 300 mPaS to 800 mPaS, or 500 mPaS to 1000 mPaS, or 800 mPaS to 1500 mPaS, or 1000 mPaS to 2000 mPaS, or 1500 mPaS to 3000 mPaS, or 2000 mPaS to 4000 mPaS, or 3000 mPaS to 6000 mPaS, or 4000 mPaS to 8000 mPaS at 0° C. In some embodiments, the viscosity is measured using a rheometer. In some embodiments, the viscosity is measured using a viscometer. In some embodiments, the rheometer comprises a temperature control unit which maintains and controls temperature at least in the range of 0.0±0.1° C.

In some embodiments, the ophthalmic compositions of the present disclosure comprise a final viscosity in the range of 1 mPaS to 5 mPaS, or 5 mPaS to 25 mPaS, or 25 mPaS to 100 mPaS, or 100 mPaS to 200 mPaS, or 150 mPaS to 300 mPaS, or 200 mPaS to 500 mPaS, or 300 mPaS to 800 mPaS, or 500 mPaS to 1000 mPaS, or 800 mPaS to 1500 mPaS, or 1000 mPaS to 2000 mPaS, or 1500 mPaS to 3000 mPaS, or 2000 mPaS to 4000 mPaS, or 3000 mPaS to 6000 mPaS, or 4000 mPaS to 8000 mPaS at 5° C. In some embodiments, the viscosity is measured using a rheometer. In some embodiments, the viscosity is measured using a viscometer. In some embodiments, the rheometer comprises a temperature control unit which maintains and controls temperature at least in the range of 5.0±0.1° C.

In some embodiments, the ophthalmic compositions of the present disclosure comprise a final viscosity in the range of 1 mPaS to 5 mPaS, or 5 mPaS to 25 mPaS, or 25 mPaS to 100 mPaS, or 100 mPaS to 200 mPaS, or 150 mPaS to 300 mPaS, or 200 mPaS to 500 mPaS, or 300 mPaS to 800 mPaS, or 500 mPaS to 1000 mPaS, or 800 mPaS to 1500 mPaS, or 1000 mPaS to 2000 mPaS, or 1500 mPaS to 3000 mPaS, or 2000 mPaS to 4000 mPaS, or 3000 mPaS to 6000 mPaS, or 4000 mPaS to 8000 mPaS at 10° C. In some embodiments, the viscosity is measured using a rheometer. In some embodiments, the viscosity is measured using a viscometer. In some embodiments, the rheometer comprises a temperature control unit which maintains and controls temperature at least in the range of 10.0±0.1° C.

In some embodiments, the ophthalmic compositions of the present disclosure comprise a final viscosity in the range of 1 mPaS to 5 mPaS, or 5 mPaS to 25 mPaS, or 25 mPaS to 100 mPaS, or 100 mPaS to 200 mPaS, or 150 mPaS to 300 mPaS, or 200 mPaS to 500 mPaS, or 300 mPaS to 800 mPaS, or 500 mPaS to 1000 mPaS, or 800 mPaS to 1500 mPaS, or 1000 mPaS to 2000 mPaS, or 1500 mPaS to 3000 mPaS, or 2000 mPaS to 4000 mPaS, or 3000 mPaS to 6000 mPaS, or 4000 mPaS to 8000 mPaS at 15° C. In some embodiments, the viscosity is measured using a rheometer. In some embodiments, the viscosity is measured using a viscometer. In some embodiments, the rheometer comprises a temperature control unit which maintains and controls temperature at least in the range of 15.0±0.1° C.

In some embodiments, the ophthalmic compositions of the present disclosure comprise a final viscosity in the range of 1 mPaS to 5 mPaS, or 5 mPaS to 25 mPaS, or 25 mPaS to 100 mPaS, or 100 mPaS to 200 mPaS, or 150 mPaS to 300 mPaS, or 200 mPaS to 500 mPaS, or 300 mPaS to 800 mPaS, or 500 mPaS to 1000 mPaS, or 800 mPaS to 1,500 mPaS, or 1000 mPaS to 2000 mPaS, or 1500 mPaS to 3000 mPaS, or 2000 mPaS to 4000 mPaS, or 3000 mPa to 6000 mPaS, or 4000 mPaS to 8000 mPaS at 20° C. In some embodiments, the viscosity is measured using a rheometer. In some embodiments, the viscosity is measured using a viscometer. In some embodiments, the rheometer comprises a temperature control unit which maintains and controls temperature at least in the range of 20.0±0.1° C.

In some embodiments, the ophthalmic compositions of the present disclosure comprise a final viscosity in the range of 1 mPaS to 5 mPaS, or 5 mPaS to 25 mPaS, or 25 mPaS to 100 mPaS, or 100 mPaS to 200 mPaS, or 150 mPaS to 300 mPaS, or 200 mPaS to 500 mPaS, or 300 mPaS to 800 mPaS, or 500 mPaS to 1000 mPaS, or 800 mPaS to 1500 mPaS, or 1000 mPaS to 2000 mPaS, or 1500 mPaS to 3000 mPaS, or 2000 mPaS to 4000 mPaS, or 3000 mPaS to 6000 mPaS, or 4000 mPaS to 8000 mPaS at 25° C. In some embodiments, the viscosity is measured using a rheometer. In some embodiments, the viscosity is measured using a viscometer. In some embodiments, the rheometer comprises a temperature control unit which maintains and controls temperature at least in the range of 25.0±0.1° C.

In some embodiments, the ophthalmic compositions of the present disclosure comprise a final viscosity in the range of 1 mPaS to 5 mPaS, or 5 mPaS to 25 mPaS, or 25 mPaS to 100 mPaS, or 100 mPaS to 200 mPaS, or 150 mPaS to 300 mPaS, or 200 mPaS to 500 mPaS, or 300 mPaS to 800 mPaS, or 500 mPaS to 1000 mPaS, or 800 mPaS to 1500 mPaS, or 1000 mPaS to 2000 mPaS, or 1500 mPaS to 3000 mPaS, or 2000 mPaS to 4000 mPaS, or 3000 mPaS to 6000 mPaS, or 4000 mPaS to 8000 mPaS at 37° C. In some embodiments, the viscosity is measured using a rheometer. In some embodiments, the viscosity is measured using a viscometer. In some embodiments, the rheometer comprises a temperature control unit which maintains and controls temperature at least in the range of 37.0±0.1° C.

In some embodiments, the ophthalmic compositions of the present disclosure comprise a final viscosity in the range of 1 mPaS to 5 mPaS, or 5 mPaS to 25 mPaS, or 25 mPaS to 100 mPaS, or 100 mPaS to 200 mPaS, or 150 mPaS to 300 mPaS, or 200 mPaS to 500 mPaS, or 300 mPaS to 800 mPaS, or 500 mPaS to 1000 mPaS, or 800 mPaS to 1500 mPaS, or 1000 mPaS to 2000 mPaS, or 1500 mPaS to 3000 mPaS, or 2000 mPaS to 4000 mPaS, or 3000 mPaS to 6000 mPaS, or 4000 mPaS to 8000 mPaS at 50° C. In some embodiments, the viscosity is measured using a rheometer. In some embodiments, the viscosity is measured using a viscometer. In some embodiments, the rheometer comprises a temperature control unit which maintains and controls temperature at least in the range of 50.0±0.1° C.

In some embodiments, the ophthalmic compositions of the present disclosure comprise a final viscosity in the range of 1 mPaS to 5 mPaS, or 5 mPaS to 25 mPaS, or 25 mPaS to 100 mPaS, or 100 mPaS to 200 mPaS, or 150 mPaS to 300 mPaS, or 200 mPaS to 500 mPaS, or 300 mPaS to 800 mPaS, or 500 mPaS to 1000 mPaS, or 800 mPaS to 1500 mPaS, or 1000 mPaS to 2000 mPaS, or 1500 mPaS to 3000 mPaS, or 2000 mPaS to 4000 mPaS, or 3000 mPaS to 6000 mPaS, or 4000 mPaS to 8000 mPaS at 75° C. In some embodiments, the viscosity is measured using a rheometer. In some embodiments, the viscosity is measured using a viscometer. In some embodiments, the rheometer comprises a temperature control unit which maintains and controls temperature at least in the range of 75.0±0.1° C.

In some embodiments, the ophthalmic compositions of the present disclosure comprise a final viscosity in the range of 1 mPaS to 5 mPaS, or 5 mPaS to 25 mPaS, or 25 mPaS to 100 mPaS, or 100 mPaS to 200 mPaS, or 150 mPaS to 300 mPaS, or 200 mPaS to 500 mPaS, or 300 mPaS to 800 mPaS, or 500 mPaS to 1000 mPaS, or 800 mPaS to 1500 mPaS, or 1000 mPaS to 2000 mPaS, or 1500 mPaS to 3000 mPaS, or 2000 mPaS to 4000 mPaS, or 3000 mPaS to 6000 mPaS, or 4000 mPaS to 8000 mPaS at 95° C. In some embodiments, the viscosity is measured using a rheometer. In some embodiments, the viscosity is measured using a viscometer. In some embodiments, the rheometer comprises a temperature control unit which maintains and controls temperature at least in the range of 95.0±0.1° C.

In some embodiments, the ophthalmic compositions of the present disclosure comprise a final viscosity in the range of 1 mPaS to 5 mPaS, or 5 mPaS to 25 mPaS, or 25 mPaS to 100 mPaS, or 100 mPaS to 200 mPaS, or 150 mPaS to 300 mPaS, or 200 mPaS to 500 mPaS, or 300 mPaS to 800 mPaS, or 500 mPaS to 1000 mPaS, or 800 mPaS to 1500 mPaS, or 1000 mPaS to 2000 mPaS, or 1500 mPaS to 3000 mPaS, or 2000 mPaS to 4000 mPaS, or 3000 mPaS to 6000 mPaS, or 4000 mPaS to 8000 mPaS at 98° C. In some embodiments, the viscosity is measured using a rheometer. In some embodiments, the viscosity is measured using a viscometer. In some embodiments, the rheometer comprises a temperature control unit which maintains and controls temperature at least in the range of 98.0±0.1° C.

In some embodiments, the ophthalmic compositions of the present disclosure comprise a final viscosity in the range of 1 mPaS to 5 mPaS, or 5 mPaS to 25 mPaS, or 25 mPaS to 100 mPaS, or 100 mPaS to 200 mPaS, or 150 mPaS to 300 mPaS, or 200 mPaS to 500 mPaS, or 300 mPaS to 800 mPaS, or 500 mPaS to 1,000 mPaS, or 800 mPaS to 1,500 mPaS, or 1000 mPaS to 2000 mPaS, or 1500 mPaS to 3000 mPaS, or 2000 mPaS to 4000 mPaS, or 3000 mPaS to 6000 mPaS, or 4000 mPaS to 8000 mPaS at 100° C. In some embodiments, the viscosity is measured using a rheometer. In some embodiments, the viscosity is measured using a viscometer. In some embodiments, the rheometer comprises a temperature control unit which maintains and controls temperature at least in the range of 100.0±0.1° C.

In some embodiments, the ophthalmic composition comprises, consists essentially of, or yet further consists of a final viscosity of at least about 1 mPaS to 5 mPaS at 25° C., or 5 mPaS to 25 mPaS at 25° C., or 25 mPaS to 100 mPaS at 25° C., or 100 mPaS to at least about 200 mPaS at 25° C., or optionally about 1 mPaS, about 5 mPaS, about 25 mPaS, about 100 mPaS, or about 150 mPaS, or about 160 mPaS, or about 170 mPaS, or about 180 mPaS, or about 190 mPaS, or about 200 mPaS, at 25° C.

In a preferred embodiment, the ophthalmic composition comprises, consists essentially of, or yet further consists of a final viscosity of at least about 5 mPaS at 25° C.

In some embodiments, the ophthalmic compositions comprises a final viscosity of at least about 500 mPaS to at least about 900 mPaS at 25° C., or optionally about 500 mPaS, or about 600 mPaS, or about 700 mPaS, or about 800 mPaS, or about 850 mPaS, or about 860 mPaS, or about 870 mPaS, or about 880 mPaS, or about 890 mPaS, or about 900 mPaS, at 25° C. In a preferred embodiment, the ophthalmic composition comprises, consists essentially of, or yet further consists of a final viscosity of at least about 867 mPaS at 25° C.

In some embodiments, the ophthalmic composition comprises, consists essentially of, or yet further consists of a final viscosity of at least about 1000 mPaS to at least about 2500 mPaS at 25° C., or optionally about 1000 mPaS, or about 1500 mPaS, or about 2000 mPaS, or about 2100 mPaS, or about 2200 mPaS, or about 2300 mPaS, or about 2400 mPaS, or about 2500 mPaS, at 25° C. In a preferred embodiment, the ophthalmic composition comprises, consists essentially of, or yet further consists of a final viscosity of at least about 2145 mPaS, at 25° C.

In some embodiments, the ophthalmic composition comprises, consists essentially of, or yet further consists of a viscosity of at least about 3000 mPaS to at least about 4000 mPaS at 25° C., or optionally about 3000 mPaS, or about 3500 mPaS, or about 3600 mPaS, about 3700 mPaS, or about 3800 mPaS, or about 3900 mPaS, or about 4000 mPaS, at 25° C. In a preferred embodiment, the ophthalmic composition comprises, consists essentially of, or yet further consists of a viscosity of at least about 3815 mPaS at 25° C.

In some embodiments, the ophthalmic compositions of the present disclosure have a viscosity of at least 1 mPaS at 25° C., and comprise, consist essentially of, or yet further consist of: a) an effective amount of one or more rifamycin compounds or a pharmaceutically acceptable salt thereof, b) a buffer solution, and c) one or more viscosity imparting agents. In some preferred embodiments, the one or more rifamycin compounds is rifampicin, and the one or more viscosity imparting agents is petrolatum. In some preferred embodiments, the one or more rifamycin compounds is rifampicin, and the one or more viscosity imparting agents is liquid paraffin.

In some embodiments, the one or more rifamycin compounds is rifampicin, the one or more viscosity imparting agents is petrolatum, and the effective amount of rifampicin comprises a final concentration in the composition of at least about 0.001% w/w. In some embodiments, the one or more rifamycin compounds is rifampicin, the one or more viscosity imparting agents is liquid paraffin, and the effective amount of rifampicin comprises a final concentration in the composition of at least about 0.01% w/w. In some embodiments, the one or more rifamycin compounds is rifampicin, the one or more viscosity imparting agents is liquid paraffin, and the effective amount of rifampicin comprises a final concentration in the composition of at least about 0.1% w/w. In some embodiments, the one or more rifamycin compounds is rifampicin, the one or more viscosity imparting agents is liquid paraffin, and the effective amount of rifampicin comprises a final concentration in the composition of at least about 0.001% w/w. In some embodiments, the one or more rifamycin compounds is rifampicin, the one or more viscosity imparting agents is petrolatum, and the effective amount of rifampicin comprises a final concentration in the composition of at least about 1% w/w. In some embodiments, the one or more rifamycin compounds is rifampicin, the one or more viscosity imparting agents is liquid paraffin, and the effective amount of rifampicin comprises a final concentration in the composition of at least about 1% w/w. In a further embodiment, the one or more rifamycin compounds is rifampicin, the one or more viscosity imparting agents is petrolatum, the effective amount of rifampicin comprises a final concentration in the composition of at least about 0.001% w/w, and the composition comprises a final viscosity of at least about 1 mPaS, about 5 mPaS, about 9 mPaS, about 55 mPaS, about 146 mPaS, about 161 mPaS, at least about 867 mPaS, at least about 2145 mPaS, or at least about 3815 mPaS at 25° C. In a further embodiment, the one or more rifamycin compounds is rifampicin, the one or more viscosity imparting agents is petrolatum, the effective amount of rifampicin comprises a final concentration in the composition of at least about 1% w/w, and the composition comprises a final viscosity of at least about 5 mPaS, about 9 mPaS, about 55 mPaS, about 146 mPaS, about 161 mPaS, at least about 867 mPaS, at least about 2145 mPaS, or at least about 3815 mPaS at 25° C. In a further embodiment, the one or more rifamycin compounds is rifampicin, the one or more viscosity imparting agents is liquid paraffin, the effective amount of rifampicin comprises a final concentration in the composition of at least about 0.001% w/w, and the composition comprises a final viscosity of at least about 1 mPaS, about 5 mPaS, about 9 mPaS, about 55 mPaS, about 146 mPaS, about 161 mPaS, at least about 867 mPaS, at least about 2145 mPaS, or at least about 3815 mPaS at 25° C. In a further embodiment, the one or more rifamycin compounds is rifampicin, the one or more viscosity imparting agents is liquid paraffin, the effective amount of rifampicin comprises a final concentration in the composition of at least about 1% w/w, and the composition comprises a final viscosity of at least about 5 mPaS, about 9 mPaS, about 55 mPaS, about 146 mPaS, about 161 mPaS, at least about 867 mPaS, at least about 2145 mPaS, or at least about 3815 mPaS at 25° C.

### Phospholipid Compounds and Preservatives

In some embodiments, the ophthalmic compositions disclosed herein comprise, consist essentially of, or yet further consist of one or more phospholipid compounds. Suitable phospholipids are known in the present technical field, and examples thereof include, but are not limited to, small alkyl chain phospholipids, phosphatidylcholine, egg phosphatidylcholine, soybean phosphatidylcholine, dipalmitoylphosphatidylcholine, soy phosphatidylglycerol, egg phosphatidylglycerol, distearoylphosphatidylglycerol, dimyristoylphosphatidylcholine, distearoylphosphatidylcholine, dilaurylphosphatidylcholine, 1-myristoyl-2-palmitoylphosphatidylcholine, 1-paimitoyl-2-myristoylphosphatidylcholine, 1-palmitoyl-2-stearoylphosphatidylcholine, 1-stearoyl-2-palmitoylphosphatidylcholine, dioleoylphosphatidylcholine, 1-palmitoyl-2-oleoylphosphatidylcholine, 1-oleoyl-2-palmitoylphosphatidylcholine, dioleoylphosphatidylethanolamine, dilauroylphosphatidylglycerol, phosphatidylserine, phosphatidylethanolamine, phosphatidylinositol, phosphatidylglycerol, diphosphatidylglycerol, dimyristoylphosphatidylglycerol, dipalmitoylphosphatidylglycerol, distearoylphosphatidylglycerol, dioleoylphosphatidylglycerol, phosphatidic acid, dimyristoylphosphatidic acid, dipalmitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, sphingomyelin, sphingolipids, brain sphingomyelin, dipalmitoylsphingomyelin, distearoylsphingomyelin, galactocerebroside, gangliosides, cerebrosides, phosphatidylglycerol, phosphatidic acid, lysolecithin, lysophosphatidylethanolamine, cephalin, cardiolipin, dicetylphosphate, distearoyl-phosphatidylethanolamine, or combinations thereof. The phospholipid can also be a derivative or analogue of any of the above-described phospholipids. In some embodiments, the ophthalmic composition comprises, consists essentially of, or yet further consists of a phospholipid compound(s) with a final concentration in the composition of about 0.00001% by weight to about 50% by weight, about 0.00005% by weight to about 50% by weight, about 0.0001% by weight to about 50% by weight, about 0.001% by weight to about 50% by weight, about 0.01% by weight to 20% by weight, about 0.1% by weight to 15% by weight, about 0.15% by weight to 10% by weight, about 0.2% by weight to 5% by weight, about 0.25% by weight to 3% by weight, about 0.3% by weight to 2% by weight, about 0.1% by weight to 20% by weight, about 1% by weight to 10% by weight, about 2% by weight to 10% by weight, about 2% by weight to 8% by weight, about 2% by weight to 5% by weight, about 5% by weight to 10% by weight, or about 5% by weight to 20% by weight. In some embodiments, the ophthalmic composition comprises, consists essentially of, or yet further consists of a final concentration of about 0.01% by weight to 10% by weight of the phospholipid compound(s).

In some embodiments, the ophthalmic compositions of the present disclosure optionally comprise, consist essentially of, or yet further consist of a preservative agent. Examples of preservative agents include, but are not limited to, imidazolidinyl urea, methylparaben, propylparaben, phenoxyethanol, disodium EDTA, benzalkonium chloride, thimerosal, chlorobutanol sorbic acid, and a combination thereof. In some embodiments, the ophthalmic composition comprises, consists essentially of, or yet further consists of a preservative agent with a final concentration in the composition of about 0.00001% by weight to about 50% by weight, about 0.00005% by weight to about 50% by weight, about 0.0001% by weight to about 50% by weight, about 0.001% by weight to about 50% by weight, about 0.01% by weight to 20% by weight, about 0.1% by weight to 15% by weight, about 0.15% by weight to 10% by weight, about 0.2% by weight to 5% by weight, about 0.25% by weight to 3% by weight, about 0.3% by weight to 2% by weight, about 0.1% by weight to 20% by weight, about 1% by weight to 10% by weight, about 2% by weight to 10% by weight, about 2% by weight to 8% by weight, about 2% by weight to 5% by weight, about 5% by weight to 10% by weight, or about 5% by weight to 20% by weight. In some embodiments, the ophthalmic composition comprises, consists essentially of, or yet further consists of a final concentration in the composition of about 0.01% by weight to 10% by weight of the preservative agent.

### Pharmaceutical Compositions

In certain aspects, an ophthalmic composition of the present disclosure can be administered alone or as a component of a pharmaceutical formulation (also referred to as a therapeutic composition or pharmaceutical composition). A pharmaceutical formulation refers to a preparation which is in such form as to permit the biological activity of an active ingredient (e.g., one or more rifamycin compounds) contained therein to be effective and which contains no additional components which are unacceptably toxic to a subject to which the formulation would be administered. The subject compounds can be formulated for administration in any convenient way for use in human or veterinary medicine. For example, one or more agents of the present disclosure can be formulated with a pharmaceutically acceptable carrier. A pharmaceutically acceptable carrier refers to an ingredient in a pharmaceutical formulation, other than an active ingredient, which is generally nontoxic to a subject. A pharmaceutically acceptable carrier includes, but is not limited to, a buffer, excipient, stabilizer, and/or preservative. In general, pharmaceutical formulations for use in the present disclosure are in a pyrogen-free, physiologically-acceptable form when administered to a subject. Therapeutically useful agents other than those described herein, which can optionally be included in the formulation as described above, can be administered in combination with the subject agents in the methods of the present disclosure.

Typically, compounds will be administered to the eye including, e.g., by topical administration, intraocular (e.g., intravitreal) injection, or by implant or device. An intravitreal injection can be injected, for example, through the pars plana, 3 mm to 4 mm posterior to the limbus. Pharmaceutical compositions for administration to the eye can be formulated in a variety of ways including, for example, eye drops, ophthalmic solutions, ophthalmic suspensions, ophthalmic emulsions, intravitreal injections, sub-Tenon injections, ophthalmic bioerodible implant, and non-bioerodible ophthalmic inserts or depots.

In some embodiments compounds will be administered parenterally [e.g., by intravenous (IV.) injection, intra-arterial injection, intraosseous injection, intramuscular injection, intrathecal injection, subcutaneous injection, or intradermal injection].

In some embodiments, the ophthalmic compositions of the present composition are formulated into a pharmaceutical compositions suitable for ocular or parenteral administration, and comprise, consist essentially of, or yet further consist of one or more rifamycin compounds in combination with one or more viscosity imparting agents. In some embodiments, the pharmaceutical compositions can be pharmaceutically acceptable sterile isotonic aqueous or nonaqueous solutions, dispersions, suspensions or emulsions, or sterile powders which can be reconstituted into sterile solutions or dispersions just prior to use. Solutions or dispersions contain antioxidants, buffers, bacteriostats, suspending agents, thickening agents, or solutes which render the formulation isotonic with the blood of the intended recipient. Examples of suitable aqueous and nonaqueous carriers which are employed in the pharmaceutical formulations of the present disclosure include water, ethanol, polyols (e.g., glycerol, propylene glycol, polyethylene glycol, etc.), vegetable oils (e.g., olive oil), injectable organic esters (e.g., ethyl oleate), and suitable mixtures thereof. Proper fluidity is maintained, for example, by the use of coating materials (e.g., lecithin), by the maintenance of the required particle size in the case of dispersions, and by the use of surfactants.

In some embodiments, a therapeutic method of the present disclosure includes administering the pharmaceutical composition systemically, or locally, from an implant or device. Further, the pharmaceutical composition is encapsulated or injected in a form for delivery to a target tissue site (e.g., bone marrow or muscle). In certain embodiments, compositions of the present disclosure include a matrix capable of delivering one or more of the agents of the present disclosure to a target tissue site (e.g., bone marrow or muscle), providing a structure for the developing tissue and optimally capable of being resorbed into the body. For example, the matrix provides slow release of one or more agents of the present disclosure. Such matrices are formed of materials presently in use for other implanted medical applications.

The choice of matrix material is based on one or more of: biocompatibility, biodegradability, mechanical properties, cosmetic appearance, and interface properties. The particular application of the subject compositions defines the appropriate formulation. Potential matrices for the compositions are biodegradable and chemically defined calcium sulfate, tricalcium phosphate, hydroxyapatite, polylactic acid, and polyanhydrides. Other potential materials are biodegradable and biologically well-defined, including, for example, bone or dermal collagen. Further matrices comprise, consist essentially of, or yet further consist of pure proteins or extracellular matrix components. Other potential matrices are non-biodegradable and chemically defined, including, for example, sintered hydroxyapatite, bioglass, aluminates, or other ceramics. Matrices are comprised of combinations of any of the above mentioned types of material including, for example, polylactic acid and hydroxyapatite or collagen and tricalcium phosphate. The bioceramics are altered in composition (e.g., calcium-aluminate-phosphate) and processing to alter one or more of pore size, particle size, particle shape, and biodegradability.

In certain embodiments, pharmaceutical compositions of the present disclosure are administered orally, for example, in the form of capsules, cachets, pills, tablets, lozenges (using a flavored basis such as sucrose and acacia or tragacanth), powders, granules, a solution or a suspension in an aqueous or non-aqueous liquid, an oil-in-water or water-in-oil liquid emulsion, or an elixir or syrup, or pastille (using an inert base, such as gelatin and glycerin, or sucrose and acacia), and/or a mouth wash, each containing a predetermined amount of a compound of the present disclosure and optionally one or more other active ingredients. A compound of the present disclosure and optionally one or more other active ingredients is also administered as a bolus, electuary, or paste.

In solid dosage forms for oral administration (e.g., capsules, tablets, pills, dragees, powders, and granules), one or more compounds of the present disclosure is mixed with one or more pharmaceutically acceptable carriers including, for example, sodium citrate, dicalcium phosphate, a filler or extender (e.g., a starch, lactose, sucrose, glucose, mannitol, and silicic acid), a binder (e.g. carboxymethylcellulose, an alginate, gelatin, polyvinyl pyrrolidone, sucrose, and acacia), a humectant (e.g., glycerol), a disintegrating agent (e.g., agar-agar, calcium carbonate, potato or tapioca starch, alginic acid, a silicate, and sodium carbonate), a solution retarding agent (e.g. paraffin), an absorption accelerator (e.g. a quaternary ammonium compound), a wetting agent (e.g., cetyl alcohol and glycerol monostearate), an absorbent (e.g., kaolin and bentonite clay), a lubricant (e.g., a talc, calcium stearate, magnesium stearate, solid polyethylene glycols, sodium lauryl sulfate), a coloring agent, and mixtures thereof. In the case of capsules, tablets, and pills, the pharmaceutical formulation (composition) also comprises, consists essentially of, or yet further consists of a buffering agent. Solid compositions of a similar type are also employed as fillers in soft and hard-filled gelatin capsules using one or more excipients including, e.g., lactose or a milk sugar as well as a high molecular-weight polyethylene glycol.

Liquid dosage forms for oral administration of the pharmaceutical composition include pharmaceutically acceptable emulsions, microemulsions, solutions, suspensions, syrups, and elixirs. In addition to the active ingredient(s), the liquid dosage form can contain an inert diluent commonly used in the art including, for example, water or other solvent, a solubilizing agent and/or emulsifier [e.g., ethyl alcohol, isopropyl alcohol, ethyl carbonate, ethyl acetate, benzyl alcohol, benzyl benzoate, propylene glycol, or 1,3-butylene glycol, an oil (e.g., cottonseed, groundnut, corn, germ, olive, castor, and sesame oil), glycerol, tetrahydrofuryl alcohol, a polyethylene glycol, a fatty acid ester of sorbitan, and mixtures thereof]. Besides inert diluents, the oral formulation also includes an adjuvant including, for example, a wetting agent, an emulsifying and suspending agent, a sweetening agent, a flavoring agent, a coloring agent, a perfuming agent, a preservative agent, and combinations thereof.

Suspensions, in addition to the active compounds, contain suspending agents including, for example, an ethoxylated isostearyl alcohol, polyoxyethylene sorbitol, a sorbitan ester, microcrystalline cellulose, aluminum metahydroxide, bentonite, agar-agar, tragacanth, and combinations thereof.

Prevention of the action and/or growth of microorganisms is ensured by the inclusion of various antibacterial and antifungal agents including, for example, paraben, chlorobutanol, and phenol sorbic acid.

In certain embodiments, it is desirable to include an isotonic agent including, for example, a sugar or sodium chloride into the compositions. In addition, prolonged absorption of an injectable pharmaceutical form is brought about by the inclusion of an agent that delays absorption, including, for example, aluminum monostearate and gelatin.

It is understood that the dosage regimen is determined by the attending physician considering various factors which modify the action of the one or more of the agents of the present disclosure. The various factors include, but are not limited to, the patient's red blood cell count, hemoglobin level, the desired target red blood cell count, the patient's age, the patient's sex, the patient's diet, the severity of any disease that can be contributing to a depressed red blood cell level, the time of administration, and other clinical factors. The addition of other known active agents to the final composition also affects the dosage. Progress is monitored by periodic assessment of one or more of red blood cell levels, hemoglobin levels, reticulocyte levels, and other indicators of the hematopoietic process.

In certain embodiments, the frequency of administration is, for example, once daily, twice daily, 3 times, 4 times, 5, 6, 7, 8, 9, or 10 times daily. In certain embodiments, once 2 days, once 3 days, 4 days, 5 days, 6 days, 7 days, 8, 9, or 10 days can be employed. In certain embodiments, once a week, 2 weeks, 3, or 4 weeks can be employed. The volume of eye drops or ointment is, for example, 3 μL/application, 5 μL/application, 10 μL/application, 20 μL/application, 30 μL/application, 50 μL/application, 60 μL/application, or 70 μL/application.

Another targeted delivery system for one or more of the agents of the present disclosure is a colloidal dispersion system. Colloidal dispersion systems include, for example, macromolecule complexes, nanocapsules, microspheres, beads, and lipid-based systems including oil-in-water emulsions, micelles, mixed micelles, and liposomes. In certain embodiments, the preferred colloidal system of this disclosure is a liposome. Liposomes are artificial membrane vesicles which are useful as delivery vehicles in vitro and in vivo. For example, RNA, DNA, and intact virions has been shown to be encapsulated within the aqueous interior and be delivered to cells in a biologically active form [see, e.g., Fraley, et al. (1981) Trends Biochem. Sci., 6:77].

The composition of the liposome is usually a combination of phospholipids, which includes a steroid (e.g. cholesterol). The physical characteristics of liposomes depends on pH, ionic strength, and the presence of divalent cations. Other phospholipids or other lipids are also used, including, for example a phosphatidyl compound (e.g., phosphatidylglycerol, phosphatidylcholine, phosphatidylserine, phosphatidylethanolamine, sphingolipid, cerebroside, or a ganglioside), egg phosphatidylcholine, dipalmitoylphosphatidylcholine, and distearoylphosphatidylcholine. The targeting of liposomes is also possible based on, for example, organ specificity, cell specificity, and organelle specificity and is known in the art.

In certain aspects, the disclosure provides pharmaceutical preparations comprising, consisting essentially of, or yet further consisting of one or more rifamycin compounds of the present disclosure and a pharmaceutically acceptable carrier. In some embodiments, a pharmaceutical preparation also comprises, consist essentially of, or yet further consist of one or more additional active agents such as a compound that is used to treat a disorder of the eye such as those described herein. Preferably, a pharmaceutical preparation of the disclosure is substantially pyrogen-free. In certain embodiments, the disclosure provides packaged pharmaceuticals comprising a pharmaceutical preparation described herein and labeled for use in one or more of increasing, treating, or preventing one or more disorders of the eye [e.g., age-related macular degeneration, juvenile macular degeneration, wet macular degeneration, dry macular degeneration, Stargardt's disease, and Best's disease), retinal vein occlusion (e.g., central retinal vein occlusion, hemi-retinal vein occlusion, branch retinal vein occlusion, and ischemic retinal vein occlusion), retinal artery occlusion (e.g., central retinal artery occlusion, hemi-retinal artery occlusion, branch retinal artery occlusion, and ischemic retinal artery occlusion), diabetic retinopathy, ischemic optic neuropathy [e.g., anterior ischemic optic neuropathy (arteritic and non-arteritic) and posterior ischemic optic neuropathy], macular telangiectasia (type I or type II), retinal ischemia (e.g., acute retinal ischemia or chronic retinal ischemia)].

The ophthalmic compositions disclosed herein are produced by methods known in the present technical field. For example, the active ingredient, namely, one or more rifamycin compounds, is dissolved in purified water, oil, or saline. A surfactant is then be added thereto and mixed therein. Further additives, such as, e.g., an isotonic agent such as sodium chloride or glycerin, a buffer agent such as sodium phosphate or sodium borate, a pH adjuster such as diluted hydrochloric acid or sodium hydroxide, a preservative agent such as potassium sorbate, and an antioxidant such as tocopherol or ascorbic acid, is optionally added to the mixture to obtain an ophthalmic composition.

The ophthalmic compositions of the present disclosure are tested for various physicochemical, in vitro, and in vivo properties. Transparency is measured using, e.g., visual and/or fluorescence-based microscopic methods. Moreover, the presence of fine particulate matter is determined in order to ensure that the ophthalmic solution is free of foreign particles. A light obscuration method and/or a microscopic method is used for counting and/or for measuring particle size. The isotonicity and pH of the composition is also tested.

The content of a drug in the ophthalmic composition of the present disclosure is evaluated by suitable analytical methods such, e.g., as UV-vis spectroscopy and HPLC. In addition, the composition are also tested for preservative agent effectiveness, stability, and effective retention period, according to standard guidelines. The present composition is further subjected to sterilization using various sterilization methods known in the present technical field.

The rifamycin compound or a pharmaceutically acceptable salt thereof present in the ophthalmic compositions of the present disclosure are stably dissolved in an aqueous solution formulation.

The term “stable” means that the one or more rifamycin compounds or a pharmaceutically acceptable salt thereof is dissolved in an aqueous solution formulation, and is absent of any precipitation or particulate matter after a period of time has passed. Examples of said period of time include 2 hours, 5 hours, 10 hours, whole day and night, 1 day, 2 days, 3 days, 1 week, 2 weeks, 3 weeks, 1 month, 2 months, 3 months, 6 months, 1 year, 2 years, and 3 years. In addition, the temperature for maintaining stability is, for example, 0° C. to 40° C. (the upper limit: 40° C.; the lower limit: 0° C.), and the temperature can be set, as appropriate, in the above temperature range. The temperature is, e.g., a constant temperature, or a constant temperature range. Specific examples of the temperature (range) for maintaining stability include 0° C., 4° C., 10° C., 15° C., 20° C., 25° C., 30° C., 35° C., 37° C., 40° C., 4° C. to 40° C., 4° C. to 37° C., 4° C. to 30° C., 4° C. to 25° C., 4° C. to 20° C., 4° C. to 10° C., 10° C. to 37° C., 10° C. to 30° C., 10° C. to 25° C., 10° C. to 20° C., 20° C. to 25° C., 20° C. to 37° C., 25° C. to 37° C., and 25° C. to 30° C.

The ophthalmic compositions of the present disclosure are expected to exhibit high affinity to mucosal tissues, including an eye. Some embodiments described herein relate to a composition, which delivers a therapeutically effective amount of drug (e.g., one or more rifamycin compounds) to the systemic circulation via the mucosa. In some embodiments, the composition of the present invention provides advantages over other forms of administration routes (e.g. oral or intravitreal administration, etc.), which include, but are not limited to, avoiding first pass metabolism of drug(s), avoiding irritation of the GI mucosa, avoiding fluctuation in drug levels, predictable and extended duration of activity, minimizing undesirable side effects, suitability for drugs with short half-life and narrow therapeutic index, maintaining steady plasma concentrations of potent drugs, greater patient compliance due to elimination of multiple doses and dosage forms (oral and systemic), no requirement for local anesthesia, no pain associated with injections, the ease of administration, suitability for self-administration, and the ease of terminating of therapy at any point in time.

In some embodiments, the pharmaceutical composition is formulated for parenteral administration. “Parenteral administration” generally refers to routes of administration other than the gastro-intestinal tract. Examples of parenteral administration include, but are not limited to, intravenous injection, intra-arterial injection, intrathecal injection (into the spinal cord), intratonsillary injection, subcutaneous injection, intramuscular injection, infusion, or implantation. Infusion can be intradermal, or subcutaneous, or through a transdermal implant. Exemplary pharmaceutical compositions for parenteral administration are disclosed in the following references which are hereby incorporated by reference: U.S. Patent Application Pub. No 2006/0287221, U.S. Pat. Nos. 5,244,925, 4,309,421, 4,158,707, and 5,164,405, all of which are hereby incorporated by reference.

Compositions formulated for parenteral administration include aqueous solutions and/or buffers commonly used for injection and/or infusion. Commonly used aqueous buffers and/or solutions include, but are not limited to sodium chloride solutions of about 0.9%, phosphate buffers, Lactated Ringer's solution, Acetated ringer's solution, phosphate buffered saline, citrate buffers, Tris buffers, histidine buffers, HEPES buffers, glycine buffers, N-glycylglycine buffers, and the like. Other pharmaceutically acceptable carriers for parenteral administration include ethanol, glycerol, propylene glycol, cyclodextrin and cyclodextrin derivatives, vegetable oils, and the like.

In some embodiments, pharmaceutical compositions for injection and/or infusion contain preservatives present in amounts that effectively prevent or reduce microbial contamination or degradation. Various agents, e.g., phenol, m-cresol, benzyl alcohol, parabens, chlorobutanol, methotrexate, sorbic acid, thimerosol, ethyl hydroxybenzoate, bismuth tribromophenate, methyl hydroxybenzoate, bacitracin, propyl hydroxybenzoate, erythromycin, 5-fluorouracil, doxorubicin, mitoxantrone, rifamycin, chlorocresol, benzalkonium chlorides, can be used to prevent or reduce contamination.

In some embodiments, sterile solutions are prepared by incorporating a crystalline form of the ophthalmic compositions disclosed herein in the required amount in the appropriate solvent with various other ingredients as described herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain methods of preparation include but are not limited to vacuum-drying and freeze-drying techniques which yield a powder of the active ingredient plus any additional desired ingredient from a previously sterile-filtered solution thereof.

In some embodiments, the pharmaceutical composition is formulated for topical and/or transdermal delivery. Compositions of the present application are formulated into preparations in liquid, semi-solid, or solid forms suitable for local or topical administration. Examples of forms suitable for topical or local administration include but are not limited to, gels, water soluble jellies, creams, lotions, suspensions, foams, powders, slurries, ointments, oils, pastes, suppositories, solutions, sprays, emulsions, saline solutions, dimethylsulfoxide (DMSO)-based solutions. In general, carriers with higher densities are capable of providing an area with a prolonged exposure to the active ingredients. In contrast, a solution formulation provides more immediate exposure of the active ingredient to the chosen area.

The pharmaceutical composition comprises, consists essentially of, or yet further consists of suitable solid or gel phase carriers, which are compounds that allow increased penetration of, or assist in the delivery of, therapeutic molecules across the stratum corneum barrier of the skin. There are many of these penetration-enhancing molecules known to those skilled in the art of topical formulations. Examples of such carriers and excipients include, but are not limited to, alcohols (e.g., ethanol), fatty acids (e.g., oleic acid), humectants (e.g., urea), glycols (e.g., propylene glycol), surfactants (e.g., isopropyl myristate and sodium lauryl sulfate), glycerol monolaurate, sulfoxides, pyrrolidones, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present application employs transdermal delivery devices (“patches”). Such transdermal patches are used to provide continuous or discontinuous infusion of the ophthalmic compositions of the present disclosure as described herein in controlled amounts, either with or without an additional agent. The construction and use of transdermal patches for the delivery of pharmaceutical agents is well known in the art. See, e.g., U.S. Pat. Nos. 5,023,252; 4,992,445; and 5,001,139; which are herein incorporated by reference.

In some embodiments, the application provides a pharmaceutical composition comprising an effective amount of the ophthalmic compositions as described herein for transdermal delivery, and a pharmaceutical excipient suitable for delivery by inhalation. Compositions for inhalation include solutions and suspensions in pharmaceutically acceptable, aqueous or organic solvents, or mixtures thereof, and powders. The liquid or solid compositions contain suitable pharmaceutically acceptable excipients as described herein. The compositions are administered by the oral or nasal respiratory route for systemic effect. In some embodiments, compositions in preferably pharmaceutically acceptable solvents are nebulized by use of inert gases. In some embodiments, nebulized solutions are inhaled directly from the nebulizing device. In other embodiments, nebulizing device are attached to a face mask tent or intermittent positive pressure breathing machine. Solution, suspension, or powder compositions are administered, preferably orally or nasally, from devices that deliver the formulation in an appropriate manner.

The pharmaceutical compositions employed in the present disclosure are formulated for intraocular (ophthalmic), rectal, sublingual, buccal, or intranasal (e.g., intrapulmonary) administration. Formulations suitable for intraocular administration include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10% particularly about 1.5% w/w. Formulations suitable for sublingual administration, typically are formulated to dissolve rapidly upon placement in the mouth, allowing the active ingredient to be absorbed via blood vessels under the tongue. Exemplary sublingual formulations include, e.g., lozenges comprising the active ingredient in a flavored basis, usually sucrose and acacia or tragacanth; pastilles comprising the active ingredient in an inert basis such as gelatin and glycerin, or sucrose and acacia; mouthwashes comprising the active ingredient in a suitable liquid carrier; orally disintegrating tablets which can, for example, disintegrate in less than 90 seconds upon placement in the mouth; and thin films. Such disintegration can be measured by an in vitro dissolution test. Formulations for buccal administration can include, e.g., buccal tablets, bioadhesive particles, wafers, lozenges, medicated chewing gums, adhesive gels, patches, films, which can be delivered as an aqueous solution, a paste, an ointment, or aerosol, to name a few. Formulations for rectal administration are presented as a suppository with a suitable base comprising for example cocoa butter or a salicylate. Formulations suitable for intrapulmonary or nasal administration can have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration are prepared according to conventional methods and are delivered with other therapeutic agents such as compounds heretofore used in the treatment or prophylaxis of cancerous infections as described below. A pharmacological formulation of the present application is administered to the patient in an injectable formulation containing any compatible carrier, such as various vehicle, adjuvants, additives, and diluents; or the metopimazine mesylate utilized in the present application is administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present application include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The formulations can be presented in unit-dose or multi-dose sealed containers, such as ampules and vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind described herein.

The compositions and formulations are, if desired, presented in a vial, container, pack, or dispenser device which contain one or more unit dosage forms containing the active ingredient. The vial, container, pack, or dispenser device for example comprises, consists essentially of, or yet further consists of metal or plastic foil, such as a blister pack. The vial, container, pack, or dispenser device or dispenser device is accompanied by instructions for administration.

Further, the composition is be encapsulated or injected in a form for delivery to a target tissue site. In certain embodiments, compositions of the present disclosure include a matrix capable of delivering one or more therapeutic compounds to a target tissue site, providing a structure for the developing tissue and optimally capable of being resorbed into the body. For example, the matrix provides slow release of the active ingredient. Such matrices are formed of materials presently in use for other implanted medical applications.

The choice of matrix material is based on biocompatibility, biodegradability, mechanical properties, cosmetic appearance and interface properties. The particular application of the subject compositions will define the appropriate formulation. Potential matrices for the compositions are biodegradable and chemically defined calcium sulfate, tricalcium phosphate, hydroxyapatite, polylactic acid and polyanhydrides. Other potential materials are biodegradable and biologically well defined, such as bone or dermal collagen. Further matrices are comprised of pure proteins or extracellular matrix components.

Other potential matrices are non-biodegradable and chemically defined, such as sintered hydroxyapatite, bioglass, aluminates, or other ceramics. Matrices are comprised of combinations of any of the above mentioned types of material, such as polylactic acid and hydroxyapatite or collagen and tricalcium phosphate. The bioceramics are altered in composition, such as in calcium-aluminate-phosphate and processing to alter pore size, particle size, particle shape, and biodegradability. Suspensions, in addition to the active compounds, contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In certain embodiments, the compositions of the disclosure also contain adjuvants, such as preservatives, wetting agents, emulsifying agents and dispersing agents. Prevention of the action of microorganisms is ensured by the inclusion of various antibacterial and antifungal agents, for example, paraben, chlorobutanol, phenol sorbic acid, and the like. It is also desirable to include isotonic agents, such as sugars, sodium chloride, and the like into the compositions. In addition, prolonged absorption of the injectable pharmaceutical form is brought about by the inclusion of agents which delay absorption, such as aluminum monostearate and gelatin.

Preparations for such pharmaceutical compositions are described in, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

### Exemplary Therapeutic Uses

As described herein, applicants have discovered that an ophthalmic administration of the ophthalmic compositions of the present disclosure surprisingly results in efficient delivery of one or more rifamycin compounds to the back of the eye (including the sub retina, sclera, retina, and/or vitreous tissues) at a sufficient concentration effective to inhibit neovascularization. Moreover, the data of the present disclosure suggests that said ophthalmic compositions also have positive effects in treating or preventing eye (ocular) disorders, particularly vascular ocular disorders including, for example, those associated with ischemia and/or vascular insufficiency.

The structural and functional integrity of the eye depends on a regular oxygen and nutrient supply. Being one of the most metabolically active tissues, the retina consumes oxygen more rapidly than other tissues in the body [Cohen et al. (1965) Biochemistry of the Retina. Orlando, Fla.: Academic Press Inc; pp. 36-50; Anderson et al. (1964) Arch Ophthalmol 72:792-795; and Ames A. (1992) Can J Physiol Pharmacol. 70 (Suppl): S158-64]. The presence of a dual circulation system makes retinal oxygenation unique [Osborne et al. (2004) Prog Retin Eye Res. 23:91-147]. The photoreceptors and the greater portion of the outer plexiform layer receive nourishment from the choriocapillaris indirectly whereas the inner retinal layers are supplied by the superficial and deep capillary plexuses formed by branches of the central artery of the retina. Inner layers of the retina are known to show highest sensitivity to hypoxic challenges [Janaky et al. (2007) Doc Ophthalmol. 114:45-51], whereas the outer retina is more resistant to a hypoxic stress [Tinjust et al. (2002) Aviat Space Environ Med. 73:1189-94].

A number of systemic and cellular responses such as glycolysis, angiogenesis, vasodilation, and erythropoiesis enable an organism to respond to hypoxia [Harris et al. (2002) Nat Rev Cancer. 2:38-47]. Many tissues are capable of inducing protective mechanisms under hypoxic-ischemic conditions, which are typically induced within minutes of onset, and are of critical importance for limiting damage [Kitagawa et al. (1990) Brain Res. 528:21-4]. However, during prolonged hypoxic conditions, these protective mechanisms are generally diminished/lost within hours of the hypoxic-ischemic insult, leading to cell death and tissue damage [Prass et al. (2003) Stroke. 34:1981-6]. Transcriptional activator hypoxia-inducible factor-1α (HIF-1α) is a master regulator of cellular O₂ homeostasis [Iyer et al. (1998) Genes Dev. 12:149-62]. Hypoxia is known to induce HIF-1α and its target genes such as vascular endothelial growth factor (VEGF) and nitric oxide synthase (NOS) in many tissues. Interestingly, overproduction of these factors, such as during prolonged hypoxia, has been implicated in cellular death in hypoxic-ischemic conditions. In addition, enhanced extracellular accumulation of glutamate and inflammatory cytokines, which occurs during prolonged hypoxia, can damage cells and tissues. Increased expression of HIF-1α, VEGF, and various isoforms of NOS has been reported in the retina following hypoxic injury [Kaur et al. (2006) Invest Ophthalmol Vis Sci. 47:1126-41; and Tezel et al. (2004) Curr Opin Ophthalmol. 15:80-4].

Retinal ganglion cells (RGCs) are particularly sensitive to acute, transient, and mild systemic hypoxic stress [Kergoat et al. (2006) Invest Ophthalmol Vis Sci. 47:5423-7]. Loss of RGCs occurs in many ophthalmic conditions such as glaucoma and diabetes (Sucher et al. (1997) Vision Res. 37:3483-93; Abu-El-Asrar et al. (2004) Invest Ophthalmol Vis Sci. 45:2760-6], hypoxia being implicated in such loss [Wax et al. (2002) Mol Neurobiol. 26:45-55; Tezel et al. (2004). Curr Opin Ophthalmol. 15:80-4; and Chen et al. (2007) Stem Cells. 25:2291-301]. Neuronal degeneration resulting from retinal hypoxia-ischemia, caused by oxygen and substrate deprivation, is partially mediated by accumulation of free oxygen radicals [Block et al. (1997) Exp Eye Res. 64:559-64; Muller et al. (1997) Exp Eye Res. 64:637-43; and Szabo et al. (1997) Clin Neurosci. 4:240-5], glutamate excitotoxicity [Kuroiwa T et al. (1985) Acta Neuropathol (Berl) 68:122-9; Osborne et al. (2004) Prog Retin Eye Res. 23:91-147; and Kaur et al. (2006) Invest Ophthalmol Vis Sci. 47:1126-41], inflammation, and disruption of the blood retinal barrier [Kuroiwa et al. (1985) Acta Neuropathol (Berl) 68:122-9; and Kaur et al. (2007) J Pathol. 212:429-39].

Hypoxia-ischemia also results in retinal vascular damage which is associated with fluid accumulation in the extracellular spaces (vasogenic edema) or intracellulary spaces (cytotoxic edema) [Marmor et al. (1999) Doc Ophthalmol. 97:239-49]. The extracellular spaces in the inner retina consist of the narrow clefts between the tightly packed cellular elements. Fluid leaking out from damaged capillaries in the inner retina accumulates in the extracellular spaces displacing the retinal cellular elements and disrupting the normal anatomy of the neuronal connections, resulting in macular edema [Hamann et al. (2005) Acta Ophthalmol Scand. 83:523-5]. Macular edema can further exacerbate retinal ischemia and well as promote increased oxidative stress and inflammation (Guex-Crosier Y. (1999) Doc Ophthalmol. 97:297-309; van Dam PS. (2002) Diabetes Metab Res Rev. 18:176-84; and Miyake et al. (2002) Sury Ophthalmol. 47: S203-8.). Increased permeability of blood-retinal barrier (BRB) resulting in fluid accumulation has been reported to contribute to retinal neuronal degeneration by compression [Antcliff et al. (1999) Semin Ophthalmol. 14:223-32; Marumo T et al. (1999) J Vasc Res. 36:510-15; and Reichenbach et al. (2007) Graefes Arch Clin Exp Ophthalmol. 245:627-36). While initially protective, excess and/or chronic production of VEGF, nitric oxide (NO), and aquaporin-4 during hypoxic-ischemic insults can cause neovascularization and dysfunction of the BRB in the inner retina, resulting in serum leakage into the retinal tissues and retinal edema. In addition to an increase in vascular permeability, ocular hypoxia has also been correlated with endothelial cell death, leukocyte plugging of vessels, and microaneurysms [Linsenmeier et al. (1998) Invest Ophthalmol Vis Sci. 39:1647-57].

Hypoxia-ischemia occurs in various ocular conditions including, for example, retinal artery/vein occlusion or thrombosis, ocular ischemic syndrome, ischemic optic neuropathy, and retinal ischemia. Hypoxia-ischemia also has been implicated in the development of glaucoma [Flammer J. (1994) ‘Sury Ophthalmol. 38 (Suppl): S3-6; Chung et al. (1999) Sury Ophthalmol. 43 (Suppl 1): S43-50; and Tezel et al. (2004) Curr Opin Ophthalmol. 15:80-4], is thought to underlie many of the sight-threatening complications of diabetic eye disease including retinal and optic nerve head neovascularization [Linsenmeier et al. (1998) Invest Ophthalmol Vis Sci. 39:1647-57], and plays a role in age-related macular degeneration [Tso et al. (1982) Ophthalmology. 89:902-15; Yanoff et al. (1984) Sury Ophthalmol. 28 (Suppl): 505-11; and Bressler et al. (2001) In: Schachat AP, editor. Retina. St. Louis, Mo.: Mosby]. Systemic causes of ocular hypoxia include the cardiovascular effects, chronic obstructive airways disease, arterial/venous obstructive conditions, [Brown et al. (1988) Int Ophthalmol. 11:239-51] Takayasu's arteritis [Shelhamer et al. (1985) Ann Intern Med. 103:121-6], hyperviscosity syndromes [Ashton et al. (1963) J Pathol Bacteriol. 86:453-61] as well as trauma (e.g., surgery or accidental damage) [Purtscher's retinopathy; Buckley et al. (1996) Postgrad Med J. 72:409-12]. Hypoxia associated with the above conditions is a common cause of visual impairment and blindness [Osborne, et al. (2004) Prog Retin Eye Res. 23:91-147].

Therefore, in certain aspects, the present disclosure provides methods, as well as compositions, for treating or preventing an vascular disorder (disease) of the eye in a patient (subject) in need thereof (particularly mammals such as rodents, cats, dogs, primates, and humans) by administering to the patient a therapeutically effective amount of an ophthalmic composition comprising, or consisting essentially of, or yet further consisting of one or more rifamycin compounds, a buffer, and a viscosity imparting agent. A vascular disorder of the eye includes, but is not limited to: macular degeneration, diabetic retinopathy, chronic glaucoma, retinal detachment, sickle cell retinopathy, age-related macular degeneration (AMD), retinal ganglion cell injury, rubeosis iritis, inflammatory disease, chronic uveitis, neoplasm, Fuchs' heterochromic iridocyclitis, neovascular glaucoma, corneal neovascularization, choroidal neovascularization, retinal neovascularization, retinal angiomatous proliferation, glaucoma, glaucoma surgery, tissue adhesion, cicatrization, tissue fibrosis, and brain damage.

In some embodiments, the disclosure provides methods for using an ophthalmic composition comprising, consisting essentially of, or yet further consisting of one or more rifamycin compounds, a buffer, and a viscosity imparting agent, to treat or prevent vascular disorders of the eye associated with ischemia. In some embodiments, the disclosure provides methods for using an ophthalmic composition comprising, consisting essentially of, or yet further consisting of one or more rifamycin compounds, a buffer, and a viscosity imparting agent, to treat or prevent an ischemic eye disease. In some embodiments, the disclosure provides methods for using an ophthalmic composition comprising, consisting essentially of, or yet further consisting of one or more rifamycin compounds, a buffer, and a viscosity imparting agent, to treat or prevent vascular disorders of the eye associated with microvasculature insufficiency. In some embodiments, the disclosure provides methods for using an ophthalmic composition comprising, consisting essentially of, or yet further consisting of one or more rifamycin compounds, a buffer, and a viscosity imparting agent, to treat or prevent an ocular microvasculature insufficiency disease. In some embodiments, the disclosure provides methods for using an ophthalmic composition comprising, consisting essentially of, or yet further consisting of one or more rifamycin compounds, a buffer, and a viscosity imparting agent, to treat or prevent vascular disorders of the eye associated with retinopathy. In some embodiments, the disclosure provides methods for using an ophthalmic composition comprising, consisting essentially of, or yet further consisting of one or more rifamycin compounds, a buffer, and a viscosity imparting agent, to treat or prevent vascular disorders of the eye associated with optic neuropathy. In some embodiments, the disclosure provides methods for using an ophthalmic composition comprising, consisting essentially of, or yet further consisting of one or more rifamycin compounds, a buffer, and a viscosity imparting agent, to treat or prevent ischemic retinopathy. In some embodiments, the disclosure provides methods for using an ophthalmic composition comprising, consisting essentially of, or yet further consisting of one or more rifamycin compounds, a buffer, and a viscosity imparting agent, to treat or prevent ischemic optic neuropathy. In some embodiments, the disclosure provides methods for using an ophthalmic composition comprising, consisting essentially of, or yet further consisting of one or more rifamycin compounds, a buffer, and a viscosity imparting agent, to treat or prevent retinopathy associated with microvasculature insufficiency. In some embodiments, the disclosure provides methods for using an ophthalmic composition comprising, consisting essentially of, or yet further consisting of one or more rifamycin compounds, a buffer, and a viscosity imparting agent, to treat or prevent optic neuropathy associated with microvasculature insufficiency. In particular, the disclosure provides methods for using an ophthalmic composition comprising, consisting essentially of, or yet further consisting of one or more rifamycin compounds, a buffer, and a viscosity imparting agent, to treat or prevent one or more diseases selected from: macular degeneration (e.g., age-related macular degeneration, juvenile macular degeneration, wet macular degeneration, dry macular degeneration, Stargardt's disease, and Best's disease), retinal vein occlusion (e.g., central retinal vein occlusion, hemi-retinal vein occlusion, branch retinal vein occlusion, and ischemic retinal vein occlusion), retinal artery occlusion (e.g., central retinal artery occlusion, hemi-retinal artery occlusion, branch retinal artery occlusion, and ischemic retinal artery occlusion), diabetic retinopathy, ischemic optic neuropathy [e.g., anterior ischemic optic neuropathy (arteritic and non-arteritic) and posterior ischemic optic neuropathy], macular telangiectasia (type I or type II), retinal ischemia (e.g., acute retinal ischemia or chronic retinal ischemia), ocular ischemic syndrome, retinal vasculitis, and retinopathy of prematurity. In some embodiments, methods and compositions disclosed herein for treating an ocular disease result in improving vision in an eye of the patient. In some embodiments, methods and compositions disclosed herein for treating an ocular disease result in increasing visual acuity in an eye of the patient. In some embodiments, methods and compositions disclosed herein for treating an ocular disease result in increasing visual field in an eye of the patient. Optionally, methods of the disclosure for treating or preventing a vascular disorder of the eye further comprises, consists essentially of, or yet further consists of administration of one or more supportive therapies for treating or preventing the disorder in addition to administration of an ophthalmic composition comprising, consisting essentially of, or yet further consisting of one or more rifamycin compounds, a buffer, and a viscosity imparting agent [e.g., surgery, laser therapy (e.g., photocoagulation), anti-angiogenic therapy [e.g., VEGF inhibitors such as bevacizumab (Avastin®), ranibizumab (Lucentis®), and Aflibercept (Eylea®)], Ca²⁺ inhibitors (e.g., flunarizine and nifedipine), cryotherapy, hyperbaric oxygenation, Na⁺ channel blockers (e.g., topiramate), iGluR antagonists (e.g., MK-801, dextromethorphan, eliprodil, and flupirtine), antioxidants (e.g., dimethylthiourea, vitamin E, alph-lipoic acid, superoxide dismutase, catalase, desferrioxamine, mannitol, allopurinol, calcium dobesilate, flupirtine, trimetazidine, and EGB-761), anti-inflammatory agents, cyclodiathermy, cyclocryotherapy, ocular filtering procedures, implantation of drainage valves, antiplatelet therapy (e.g., aspirin, ticlopidine, and clopidogrel), anticoagulant therapy (e.g., warfarin and heparin), steroids, systemic or local corticosteroids (e.g., prednisone triamcinolone (Triesence®), and dexamethasone (Ozurdex®), steroid-sparing immunosuppressants (e.g., cyclosporine, azathioprine, cyclophosphamide, mycophenolate, mofetil, infliximab and etanercept), dietary supplements (e.g., vitamin C, vitamin E, lutein, zeaxanthin, zinc, folic acid, vitamins B6, vitamin B12, and zeaxanthin), vitrectomy, scleral buckle surgery, and pneumatic retinopexy]. In a preferred embodiment, the above methods and compositions for treating or preventing a vascular disorder of the eye in a patient in need thereof comprise, consist essentially of, or yet further consist of an ophthalmic composition that has a viscosity of at least 1 mPaS at 25° C. In a further preferred embodiment, the one or more rifamycin compounds is rifampicin, the one or more viscosity imparting agents is petrolatum or liquid paraffin, and the effective amount of rifampicin comprises a final concentration in the composition of at least about 0.001% w/w to about 1% w/w.

In certain aspects, the present disclosure provides methods and compositions for improving vision (e.g., increasing visual acuity and or visual field) in a patient in need thereof by administering to the patient a therapeutically effective amount of an ophthalmic composition comprising, consisting essentially of, or yet further consisting of one or more rifamycin compounds, a buffer, and a viscosity imparting agent. In some embodiments, the disclosure provides methods for using an ophthalmic composition comprising, consisting essentially of, or yet further consisting of one or more rifamycin compounds, a buffer, and a viscosity imparting agent, to improve vision (e.g., increase visual acuity and or visual field) in a patient with a vascular disorder of the eye. In some embodiments, the disclosure provides methods for using an ophthalmic composition comprising, consisting essentially of, or yet further consisting of one or more rifamycin compounds, a buffer, and a viscosity imparting agent, to improve vision (e.g., increase visual acuity and or visual field) in a patient with a vascular disorder of the eye associated with ischemic ocular disease. In some embodiments, the disclosure provides methods for using an ophthalmic composition comprising, consisting essentially of, or yet further consisting of one or more rifamycin compounds, a buffer, and a viscosity imparting agent, to improve vision (e.g., increase visual acuity and or visual field) in a patient with a vascular disorder of the eye associated with microvasculature insufficiency. In some embodiments, the disclosure provides methods for using an ophthalmic composition comprising, consisting essentially of, or yet further consisting of one or more rifamycin compounds, a buffer, and a viscosity imparting agent, to improve vision (e.g., increase visual acuity and or visual field) in a patient with ocular microvasculature insufficiency disease. In some embodiments, the disclosure provides methods for using an ophthalmic composition comprising, consisting essentially of, or yet further consisting of one or more rifamycin compounds, a buffer, and a viscosity imparting agent, to improve vision (e.g., increase visual acuity and or visual field) in a patient with a vascular disorder of the eye associated with retinopathy. In some embodiments, the disclosure provides methods for using an ophthalmic composition comprising, consisting essentially of, or yet further consisting of one or more rifamycin compounds, a buffer, and a viscosity imparting agent, to improve vision (e.g., increase visual acuity and or visual field) in a patient with a vascular disorder of the eye associated with optic neuropathy. In some embodiments, the disclosure provides methods for using an ophthalmic composition comprising, consisting essentially of, or yet further consisting of one or more rifamycin compounds, a buffer, and a viscosity imparting agent, to improve vision (e.g., increase visual acuity and or visual field) in a patient with ischemic retinopathy. In some embodiments, the disclosure provides methods for using an ophthalmic composition comprising, consisting essentially of, or yet further consisting of one or more rifamycin compounds, a buffer, and a viscosity imparting agent, to improve vision (e.g., increase visual acuity and or visual field) in a patient with ischemic optic neuropathy. In some embodiments, the disclosure provides methods for using an ophthalmic composition comprising, consisting essentially of, or yet further consisting of one or more rifamycin compounds, a buffer, and a viscosity imparting agent, to improve vision (e.g., increase visual acuity and or visual field) in a patient with retinopathy associated with microvasculature insufficiency. In some embodiments, the disclosure provides methods for using an ophthalmic composition comprising, consisting essentially of, or yet further consisting of one or more rifamycin compounds, a buffer, and a viscosity imparting agent, to improve vision (e.g., increase visual acuity and or visual field) in a patient with optic neuropathy associated with microvasculature insufficiency. In particular, the disclosure provides methods for using an ophthalmic composition comprising, consisting essentially of, or yet further consisting of one or more rifamycin compounds, a buffer, and a viscosity imparting agent, to improve vision (e.g., increase visual acuity and or visual field) in a patient with one or more diseases selected from: macular degeneration (e.g., age-related macular degeneration, juvenile macular degeneration, wet macular degeneration, dry macular degeneration, Stargardt's disease, and Best's disease), retinal vein occlusion (e.g., central retinal vein occlusion, hemi-retinal vein occlusion, branch retinal vein occlusion, and ischemic retinal vein occlusion), retinal artery occlusion (e.g., central retinal artery occlusion, hemi-retinal artery occlusion, branch retinal artery occlusion, and ischemic retinal artery occlusion), diabetic retinopathy, ischemic optic neuropathy [e.g., anterior ischemic optic neuropathy (arteritic and non-arteritic) and posterior ischemic optic neuropathy], macular telangiectasia (type I or type II), retinal ischemia (e.g., acute retinal ischemia or chronic retinal ischemia), ocular ischemic syndrome, retinal vasculitis, and retinopathy of prematurity. In some embodiments, the disclosure provides methods for using an ophthalmic composition comprising, consisting essentially of, or yet further consisting of one or more rifamycin compounds, a buffer, and a viscosity imparting agent, to improve vision (e.g., increase visual acuity and or visual field) in a patient with anemia. In some embodiments, the disclosure provides methods for using an ophthalmic composition comprising, consisting essentially of, or yet further consisting of one or more rifamycin compounds, a buffer, and a viscosity imparting agent, to improve vision (e.g., increase visual acuity and or visual field) in a patient with myelodysplastic syndrome. In some embodiments, the disclosure provides methods for using an ophthalmic composition comprising, consisting essentially of, or yet further consisting of one or more rifamycin compounds, a buffer, and a viscosity imparting agent, to improve vision (e.g., increase visual acuity and or visual field) in a patient with sideroblastic anemia. In some embodiments, the disclosure provides methods for using an ophthalmic composition comprising, consisting essentially of, or yet further consisting of one or more rifamycin compounds, a buffer, and a viscosity imparting agent, to improve vision (e.g., increase visual acuity and or visual field) in a patient with a hemoglobinopathy. In some embodiments, the disclosure provides methods for using an ophthalmic composition comprising, consisting essentially of, or yet further consisting of one or more rifamycin compounds, a buffer, and a viscosity imparting agent, to improve vision (e.g., increase visual acuity and or visual field) in a patient with thalassemia. In some embodiments, the disclosure provides methods for using an ophthalmic composition comprising, consisting essentially of, or yet further consisting of one or more rifamycin compounds, a buffer, and a viscosity imparting agent, to improve vision (e.g., increase visual acuity and or visual field) in a patient with sickle-cell disease. Optionally, methods of the disclosure for improving vision (e.g., increasing visual acuity and or visual field) in a patient with an ocular disease further comprises administration of one or more supportive therapies for treating or preventing the ocular disease in addition to administration of an ophthalmic composition comprising, consisting essentially of, or yet further consisting of one or more rifamycin compounds, a buffer, and a viscosity imparting agent. In a preferred embodiment, the above methods and compositions for improving vision in a patient in need thereof comprise, consist essentially of, or yet further consist of an ophthalmic composition that has a viscosity of at least 50 mPaS at 25° C. In a further preferred embodiment, the one or more rifamycin compounds is rifampicin, the one or more viscosity imparting agents is petrolatum or liquid paraffin, and the effective amount of rifampicin comprises a final concentration in the composition of at least about 0.001% w/w to at least about 1% w/w.

Macular degeneration results in loss of vision in the center of the visual field (the macula) and generally is caused by damage to the retina [de Jong P T (2006) N Engl J Med 255 (14): 1474-1485]. It is a major cause of blindness and visual impairment and usually occurs in older adults, afflicting around 20-50 million people globally. As it predominantly manifests in older adults, macular degeneration is often referred to as age-related macular degeneration (AMD). In younger patients, macular degeneration is often referred to as juvenile macular degeneration, which is generally the result of an underlying genetic disorder (e.g., Stargardt's disease or Best's disease) [Dryja et al. (1998) Science 279 (5354): 1107]. In general, macular degeneration manifest as either “dry” (non-exudative) or “wet” (exudative) disease. In dry macular degeneration, yellow deposits (drusen) accumulate in the macular, between the retinal pigment epithelium and the underlying choroid. Large and/or numerous drusen depositions disrupt the pigmented cell layer under the macula, which causes vision loss due to damaged photoreceptors (cones and rods). In general, wet macular degeneration results from abnormal blood vessel growth (choroidal neovascularization) from the choriocapillaris through the Bruch's membrane. These new vessels are fragile, leading to blood and protein leakage below the macula. Bleeding and scarring from these blood vessels can damage the photoreceptors and thus promote vision loss.

Unfortunately, there are limited treatments for dry macular degeneration. However, a large scientific study (The Age-Related Eye Disease Study 2) showed that, among people at high risk for developing late-stage macular degeneration, taking dietary supplements of vitamin C, vitamin E, lutein, and zeaxanthin in combination with zinc lowered progression to advance stages of the disease by at least 25% [Chew et al. (2013) Ophthalmology 120 (8): 1604-1611]. Another large study in women showed benefits from taking folic acid and vitamins B6 and B12 [Christen et al. (2009) Arch Intern Med 169 (4): 335-341]. Other studies have shown that lutein and zeaxanthin reduce risk of developing dry macular degeneration [Chew et al. (2013) Ophthalmology 131 (7): 843-850].

The most common therapy for wet macular degeneration is administration of one or more vascular endothelial growth factor (VEGF) antagonists (inhibitors) including, for example, bevacizumab, ranibizumab, and aflibercept. Bevacizumab (Avastin®) is humanized, monoclonal VEGF-A antibody. Similarly, ranibizumab (Lucentis®) is a monoclonal VEGF-A antibody fragment (Fab). Aflibercept (Eylea®) is an immunoglobulin Fc fusion protein comprising portions from the extracellular domains of human VEGF receptors 1 and 2. Although most cases are treated with medication, surgery or laser therapy can also be used to treat wet macular degeneration. In laser therapy, a focused beam of light is used to destroy abnormal blood vessels in the retina, preventing further aberrant vascular growth and leakage. In some cases, wet macular degeneration can be treated with photodynamic therapy, which uses a combination of a light-activated drug (photosensitizer) and a low-power laser. The photosensitive drug is injected into the patient and travels throughout the body, including in the abnormal vessels behind the eye. The low-powered laser is targeted directly on the abnormal vessels to activate the drug and thereby specifically damage the unwanted blood vessels.

In certain aspects, the present disclosure provides methods and compositions for treating or preventing macular degeneration in a patient in need thereof by administering an ophthalmic composition comprising, consisting essentially of, or yet further consisting of one or more rifamycin compounds, a buffer, and a viscosity imparting agent. In some embodiments, an ophthalmic composition comprising, consisting essentially of, or yet further consisting of one or more rifamycin compounds, a buffer, and a viscosity imparting agent, can be used to treat or prevent one or more of: age-related macular degeneration, juvenile macular degeneration, Stargardt's disease, Best's disease, dry macular degeneration, and wet macular degeneration. In some embodiments, an ophthalmic composition comprising, consisting essentially of, or yet further consisting of one or more rifamycin compounds, a buffer, and a viscosity imparting agent, can be used to treat or prevent one or more complication of macular degeneration including, for example, druse deposition/accumulation, macular edema, and neovacuolization. In some embodiments, an ophthalmic composition comprising, consisting essentially of, or yet further consisting of one or more rifamycin compounds, a buffer, and a viscosity imparting agent, can be used to improve vision (e.g., increase visual acuity and/or increase visual field) in a patient with macular degeneration. Optionally, patients afflicted with macular degeneration are to be treated with one or more supportive therapies [e.g., a VEGF antagonist (e.g., bevacizumab, ranibizumab, and aflibercept), surgery, laser therapy, photodynamic therapy, and/or dietary supplements (e.g., vitamin C, vitamin E, lutein, zeaxanthin, zinc, folic acid, vitamins B6, vitamin B12, and zeaxanthin)] for treating macular degeneration in addition to an ophthalmic composition comprising, consisting essentially of, or yet further consisting of one or more rifamycin compounds, a buffer, and a viscosity imparting agent. In a preferred embodiment, the above methods and compositions for treating or preventing macular degeneration in a patient in need thereof comprise, consist essentially of, or yet further consist of an ophthalmic composition that has a viscosity of at least 1 mPaS at 25° C. In a further preferred embodiment, the one or more rifamycin compounds is rifampicin, the one or more viscosity imparting agents is petrolatum or liquid paraffin, and the effective amount of rifampicin comprises a final concentration in the composition of at least about 0.001% w/w to 1% w/w.

Subjects with diabetes suffer life-limiting and life-threatening complications not limited to macrovascular-related stroke, ischemic heart disease, and peripheral artery disease and/or microvascular-related retinopathy, neuropathy, and nephropathy. Diabetic retinopathy is the most common microvascular complication of diabetes, and is a burgeoning global health issue (Antonetti D A, et al. N Engl J Med. 2012; 366 (13): 1227-1239.). Microvascular lesions have been utilized as the major criteria for evaluating and classifying the retina in diabetic retinopathy. Diabetic retinopathy falls into 2 broad categories: early nonproliferative diabetic retinopathy (NPDR) and advanced proliferative diabetic retinopathy (PDR) (Stitt A W, et al. Prog Retin Eye Res. 2016; 51:156-186.). Classification of NPDR is based on clinical findings manifested by visible features, including micro-aneurysms, retinal hemorrhages, intraretinal microvascular abnormalities (IRMA), and venous caliber changes. Classification of PDR is based upon the presence of pathologic preretinal neovascularization. An additional categorization in diabetic retinopathy is diabetic macular edema (DME), which occurs across both NPDR and PDR severity levels and represents the most common cause of vision loss in patients. DME arises from diabetes-induced breakdown of the blood-retinal barrier (BRB), with consequent vascular leakage of fluid and circulating proteins into the neural retina. The extravasation of fluid into the neural retina leads to abnormal retinal thickening and often cystoid edema of the macula. Systemic features of diabetes, such as hyperglycemia, dyslipidemia, and hypertension also influence the development of diabetic retinopathy.

Intraocular treatment modalities for diabetic retinopathy include laser photocoagulation, intravitreous injections of anti-VEGF and steroid agents, and vitreoretinal surgery. Current therapeutic paradigms focus on treatment of advanced disease, once PDR or DME has developed (Duh E J, et al. JCI Insight. 2017 Jul. 20; 2 (14): e93751), however, these current therapeutic modalities focus on treatment of advanced disease, once PDR or DME has developed (Duh E J, et al. JCI Insight. 2017 Jul. 20; 2 (14): e93751).

In certain aspects, the present disclosure provides methods and compositions for treating or preventing diabetic retinopathy in a patient in need thereof by administering an ophthalmic composition comprising, consisting essentially of, or yet further consisting of one or more rifamycin compounds, a buffer, and a viscosity imparting agent. In some embodiments, an ophthalmic composition comprising, consisting essentially of, or yet further consisting of one or more rifamycin compounds, a buffer, and a viscosity imparting agent, can be used to treat or prevent one or more of: diabetic retinopathy, nonproliferative diabetic retinopathy (NPDR), proliferative diabetic retinopathy (PDR), and/or diabetic macular edema (DME). In some embodiments, an ophthalmic composition comprising, consisting essentially of, or yet further consisting of one or more rifamycin compounds, a buffer, and a viscosity imparting agent, can be used to treat or prevent one or more complication of diabetic retinopathy including, for example, vitreous hemorrhage, retinal detachment, glaucoma, blindness, blurred vision, fluctuating vision, and/or macular edema. In some embodiments, an ophthalmic composition comprising, consisting essentially of, or yet further consisting of one or more rifamycin compounds, a buffer, and a viscosity imparting agent, can be used to improve vision (e.g., increase visual acuity and/or increase visual field) in a patient with diabetic retinopathy. Optionally, patients afflicted with diabetic retinopathy are to be treated with one or more supportive therapies [e.g., a VEGF antagonist (e.g., bevacizumab, ranibizumab, and aflibercept), surgery, laser therapy, photodynamic therapy, and/or dietary supplements (e.g., vitamin C, vitamin E, lutein, zeaxanthin, zinc, folic acid, vitamins B6, vitamin B12, and zeaxanthin)] for diabetic retinopathy in addition to an ophthalmic composition comprising, consisting essentially of, or yet further consisting of one or more rifamycin compounds, a buffer, and a viscosity imparting agent. In a diabetic retinopathy in a patient in need thereof comprise, consist essentially of, or yet further consist of an ophthalmic composition that has a viscosity of at least 1 mPaS at 25° C. In a further preferred embodiment, the one or more rifamycin compounds is rifampicin, the one or more viscosity imparting agents is petrolatum, liquid paraffin, light liquid paraffin, or sesame oil and the effective amount of rifampicin comprises a final concentration in the composition of at least about 0.001% w/w to 1% w/w.

In some embodiments of the methods and compositions disclosed herein, the ophthalmic composition has a viscosity of at least 1 mPaS at 25° C., and comprises, consists essentially of, or yet further consists of: a) an effective amount of one or more rifamycin compounds or a pharmaceutically acceptable salt thereof, b) a buffer solution, and c) one or more viscosity imparting agents. In some preferred embodiments, the one or more rifamycin compounds is rifampicin, and the one or more viscosity imparting agents is petrolatum. In some preferred embodiments, the one or more rifamycin compounds is rifampicin, and the one or more viscosity imparting agents is liquid paraffin. In some embodiments, the one or more rifamycin compounds is rifampicin, the one or more viscosity imparting agents is petrolatum, and the effective amount of rifampicin comprises a final concentration in the composition of at least about 0.001% w/w. In some embodiments, the one or more rifamycin compounds is rifampicin, the one or more viscosity imparting agents is liquid paraffin, and the effective amount of rifampicin comprises a final concentration in the composition of at least about 0.001% w/w. In some embodiments, the one or more rifamycin compounds is rifampicin, the one or more viscosity imparting agents is petrolatum, and the effective amount of rifampicin comprises a final concentration in the composition of at least about 1% w/w. In some embodiments, the one or more rifamycin compounds is rifampicin, the one or more viscosity imparting agents is liquid paraffin, and the effective amount of rifampicin comprises a final concentration in the composition of at least about 1% w/w. In a further embodiment, the one or more rifamycin compounds is rifampicin, the one or more viscosity imparting agents is petrolatum, the effective amount of rifampicin comprises a final concentration in the composition of at least about 0.001% w/w, and the composition comprises a final viscosity of at least about 1 mPaS, at least about 9 mPaS, at least about 55 mPaS, at least about 146 mPaS, about 161 mPaS, at least about 867 mPaS, at least about 2145 mPaS, or at least about 3815 mPaS at 25° C. In a further embodiment, the one or more rifamycin compounds is rifampicin, the one or more viscosity imparting agents is petrolatum, the effective amount of rifampicin comprises a final concentration in the composition of at least about 1% w/w, and the composition comprises a final viscosity of at least about 1 mPaS, at least about 9 mPaS, at least about 55 mPaS, at least about 146 mPaS, about 161 mPaS, at least about 867 mPaS, at least about 2145 mPaS, or at least about 3815 mPaS at 25° C. In a further embodiment, the one or more rifamycin compounds is rifampicin, the one or more viscosity imparting agents is liquid paraffin, the effective amount of rifampicin comprises a final concentration in the composition of at least about 0.001% w/w, and the composition comprises a final viscosity of about 1 mPaS, at least about 9 mPaS, at least about 55 mPaS, at least about 146 mPaS, at least about 161 mPaS, at least about 867 mPaS, at least about 2145 mPaS, or at least about 3815 mPaS at 25° C. In a further embodiment, the one or more rifamycin compounds is rifampicin, the one or more viscosity imparting agents is liquid paraffin, the effective amount of rifampicin comprises a final concentration in the composition of at least about 1% w/w, and the composition comprises a final viscosity of about 1 mPaS, at least about 9 mPaS, at least about 55 mPaS, at least about 146 mPaS, at least about 161 mPaS, at least about 867 mPaS, at least about 2145 mPaS, or at least about 3815 mPaS at 25° C.

In certain aspects, the disclosure provides a method for delivering one or more rifamycin compounds to the eye. In some embodiments, the method comprises, consists essentially of, or yet further consists of topically administering an ophthalmic composition comprising, consisting essentially of, or yet further consisting of one or more rifamycin compounds, a buffer, and a viscosity imparting agent to the eye. In some embodiments, the disclosure provides a method for delivering one or more rifamycin compounds to the sub retina, sclera, retina, and/or vitreous tissues, the method comprising topically administering an ophthalmic composition comprising, consisting essentially of, or yet further consisting of one or more rifamycin compounds, a buffer, and a viscosity imparting agent to the eye. In some embodiments, the disclosure provides a method for treating a neovascular eye disease, the method comprising topically administering an ophthalmic composition comprising, consisting essentially of, or yet further consisting of one or more rifamycin compounds, a buffer, and a viscosity imparting agent to the eye to the eye. In some embodiments, administration of the composition results in delivery of the one or more rifamycin compounds to the sub retina, sclera, retina, and/or vitreous tissues. In some embodiments, administration of the composition results in delivery of the one or more rifamycin compounds to the sub retina and sclera. In some embodiments, administration of the composition inhibits neovascularization in sub-retina tissues. In some embodiments, topical administration of the composition results in at least about a 5-fold, 10-fold, 50-fold, 100-fold, 500-fold, 1,000-fold, or 5,000-fold reduction in plasma exposure of the one or more rifamycin compounds relative to oral dosing at 300 mg. In some embodiments, topical administration of the composition results in at least about 100-fold reduction in plasma exposure of the one or more rifamycin compounds relative to oral dosing at 300 mg. In some embodiments, the composition is administered topically in a single dose per eye. In some further embodiments, the volume of the composition administered in a single dose per eye, herein referred to as composition “target dose volume per eye”, is in the range of about 2 μL to about 50 μL. In further preferred embodiments, the target dose volume per eye is in the range of about 5 μL to about 25 μL. In some further preferred embodiments, the target dose volume per eye is in the range of about 5 μL to 15 μL. In some embodiments, the neovascular eye disease is selected from the group consisting of macular degeneration, diabetic retinopathy, chronic glaucoma, retinal detachment, sickle cell retinopathy, age-related macular degeneration (AMD), retinal ganglion cell injury, rubeosis iritis, inflammatory disease, chronic uveitis, neoplasm, Fuchs' heterochromic iridocyclitis, neovascular glaucoma, corneal neovascularization, choroidal neovascularization, retinal neovascularization, retinal angiomatous proliferation, glaucoma, glaucoma surgery, tissue adhesion, cicatrization, tissue fibrosis, and brain damage. In some embodiments, the neovascular eye disease is AMD. In some embodiments, the neovascular eye disease is dry AMD. In some embodiments, the neovascular eye disease is wet AMD. In a preferred embodiment, the above methods comprise, consist essentially of, or yet further consist of an ophthalmic composition that has a viscosity of at least 1 mPaS at 25° C. In a further preferred embodiment, the one or more rifamycin compounds is rifampicin, the one or more viscosity imparting agents is petrolatum or liquid paraffin, and the effective amount of rifampicin comprises a final concentration in the composition of at least about 0.001% w/w to at least about 1% w/w.

Administration of a pharmaceutical composition according to the exemplary therapeutic uses described herein are performed by any method that enables delivery of the active ingredient (e.g., one or more rifamycin compounds) to the site of action. The composition is administered ophthalmically, topically, orally, parenterally, enterally, intraperitoneally, transdermally, intranasally, locally, non-orally, via spray, subcutaneously, intravenously, intratonsillary, intramuscularly, buccally, sublingually, rectally, intra-arterially, by infusion, or intrathecally. In some embodiments, the composition is administered ophthalmically. In some embodiments, the composition is administered topically. In some embodiments, the composition is administered systemically. In some embodiments, the composition is administered via intravitreal injection. In some embodiments, the composition is administered orally. In some embodiments, the composition is administered subcutaneously. In some embodiments, the oral administration comprises, consists essentially of, or yet further consists of administration of any of the oral dosage forms as described herein. The effective amount of the active ingredient (e.g., one or more rifamycin compounds) administered will be dependent on the subject being treated, the severity of the disorder or condition, the rate of administration, the disposition of the active ingredient (e.g., one or more rifamycin compounds) and the discretion of the prescribing physician.

In some embodiments, the methods of the present disclosure comprise administering to the subject an effective dose of one or more rifamycin compounds or compositions as disclosed herein. As used herein, an effective dose of one or more rifamycin compounds or compositions as disclosed herein is the dose required to produce a protective response in the subject to be administered. A protective response in the present context is one that prevents or ameliorates disease in a subject. The one or more rifamycin compounds or compositions as disclosed herein can be administered one or more times. The pharmaceutically effective dose depends on the type of disease, the composition used, the route of administration, the subject being treated, the physical characteristics of the subject under consideration, concurrent medication, and other factors that those skilled in the medical arts will recognize. Generally, an amount between 0.1 mg/kg and 100 mg/kg body weight of active ingredients is administered dependent upon potency of the formulated composition. In some embodiments, the one or more rifamycin compounds or compositions as disclosed herein is administered at an effective dose of one or more rifamycin compounds of at least 0.01 mg/kg, at least 0.02 mg/kg, at least 0.07 mg/kg, at least 0.1 mg/kg, at least 0.2 mg/kg, at least 0.7 mg/kg, at least 2 mg/kg, at least 7 mg/kg, or at least 20 mg/kg. In some embodiments, the one or more rifamycin compounds or compositions as disclosed herein is administered at an effective dose of one or more rifamycin compounds of at least 0.7 mg/kg. In some embodiments, an effective dose of one or more rifamycin compounds or compositions as disclosed herein is administered once daily, twice daily, 3 times daily, 4 times daily, 5 times daily, 6 times daily, 7 times daily, or 8 times daily. In some embodiments, an effective dose of one or more rifamycin compounds or compositions as disclosed herein is administered at an interval of at least 1 day, 2 days, 3 days, 4 days, 7 days, at least 14 days, at least 21 days, at least 28 days, at least 35 days, at least 42 days, at least 49 days, at least 56 days, or at least 64 days.

### EXEMPLIFICATION

The invention now being generally described, it will be more readily understood by reference to the following examples, which are included merely for purposes of illustration of certain embodiments and embodiments of the present invention, and are not intended to limit the invention.

### Example 1: Eye Drop Formulations Comprising Rifamycin Compounds

An active ingredient, namely, rifampicin, rifabutin, rifapentine, rifalazil or rifaximin is dissolved in a saline or water, and a surfactant such as polysorbate 80, Tween 80 or Tween 20 is added to and mixed with the solution. Further, various additives such as glycerin, xanthan gum, hydroxypropylmethyl cellulose (HPMC), a cyclodextrin derivative such as hydroxypropyl-β-cyclodextrin, an isotonic agent such as sodium chloride, potassium chloride or sodium bisulfate, a preservative agent such as disodium EDTA or methylparaben, and an antioxidant such as ascorbic acid are optionally added to and mixed with the solution to form a transparent solution. The solution thus obtained is filtered to remove fine particulate matters, and the pH is then adjusted by adding an acid such as hydrochloric acid or a base such as sodium chloride, thereby obtaining a desired pH value.

### Example 2: Formulations Comprising Rifamycin Compounds

As disclosed in TABLES 1-3, 16 types of formulations were prepared for use as topical eye drops comprising rifampicin. Formulations Ex. 1A (TABLE 1) and Ex. 6A (TABLE 2) were used in the following studies.

The following eye drop formulations were prepared at room temperature (TABLES 1 and 2). Rifampicin was added to the respective eye drop formulations to obtain the final concentrations as listed below.

TABLE 1

| | Ex. 1A | Ex. 2A | Ex. 3A | Ex. 4A | Ex. 5A |
|---|---|---|---|---|---|
| pH of added buffer solution | | | | pH 8.7 | pH 8.7 |
| (50 mM boric acid-borax) | | | | | |
| pH of added buffer solution | pH 8.3 | pH 8.3 | pH 8.3 | | |
| (100 mM boric acid-NaOH) | | | | | |
| Final concentration of added | 10 mM | 10 mM | 20 mM | 25 mM | 25 mM |
| buffer solution | | | | | |
| Final pH detected after addition | pH 7.64 | | pH 7.37 | pH 8.01 | pH 8.30 |
| of rifampicin | | | | | |
| NaCl* | 150 mM | 150 mM | 0.9% | 0.9% | 0.9% |
| Tween 80* | 0.5% | 0.5% | 0.5% | 0.5% | 0.5% |
| EDTA* | 0.1% | 0.1% | 0.1% | 0.1% | 0.1% |
| Benzalkonium chloride* | 0.01% | 0.01% | 0.01% | 0.01% | 0.01% |
| Rifampicin* | 0.25% | 0.5% | 0.5% | 1% | 0.5% |

*Final Concentration

TABLE 2

| | Ex. 6A | Ex. 7A | Ex. 8A | Ex. 9A | Ex. 10A |
|---|---|---|---|---|---|
| pH of added buffer solution | pH 8.7 | pH 8.7 | pH 8.5 | pH 8.5 | pH 8.3 |
| (50 mM boric acid-borax) | | | | | |
| Final concentration of added | 18 mM | 14 mM | 25 mM | 18 mM | 25 mM |
| buffer solution | | | | | |
| Final pH detected after addition | pH 8.01 | pH 8.02 | pH 7.81 | pH 7.69 | pH 7.47 |
| of rifampicin | | | | | |
| NaCl* | 0.9% | 0.9% | 0.9% | 0.9% | 0.9% |
| Tween 80* | 0.5% | 0.5% | 0.5% | 0.5% | 0.5% |
| EDTA* | 0.1% | 0.1% | 0.1% | 0.1% | 0.1% |
| Benzalkonium chloride* | 0.01% | 0.01% | 0.01% | 0.01% | 0.01% |
| Rifampicin* | 0.5% | 0.5% | 0.5% | 0.5% | 0.5% |

*Final Concentration

Rifampicin was completely dissolved at room temperature in the eye drop formulations of Ex. 1A and Ex. 5A to Ex. 8A listed in TABLES 1 and 2. Rifampicin was not precipitated in these formulations for several weeks. Rifampicin solutions were preserved at room temperature or in a refrigerator.

The buffer solutions, which were added in Ex. 1A to Ex. 10A, inclusive, were 50 mM boric acid-borax or 100 mM boric acid-NaOH. The pH values of these added buffer solutions were listed in TABLES 1 and 2. Liquid formulations were prepared by mixing the structural ingredients listed in the tables with one another, and powders of rifampicin were then added into these formulations, followed by mixing for 2 to 3 hours. Rifampicin was mixed into the formulations, and the pH values were then measured. The obtained pH values are listed in TABLE 1-3.

TABLE 3

| | Ex. 11A | | Ex. 12A | | Ex. 13A | | Ex. 14A | | Ex. 15A | | Ex. 16A | |
|---|---|---|---|---|---|---|---|---|---|---|---|---|
| pH of added buffer solution | | | | | | | | | pH 8.7 | | pH 8.7 | |
| (50 mM boric acid-borax) | | | | | | | | | | | | |
| pH of added buffer solution | pH 8.3 | | pH 8.3 | | pH 8.3 | | pH 8.3 | | | | | |
| (100 mM boric acid-NaOH) | | | | | | | | | | | | |
| Final concentration of added | 10 | mM | 10 | mM | 10 | mM | 10 | mM | 18 | mM | 18 | mM |
| buffer solution | | | | | | | | | | | | |
| Final pH detected after | pH 8.03 | | pH 8.10 | | pH 4.07 | | pH 3.32 | | pH 5.23 | | pH 3.67 | |
| addition of all structural | | | | | | | | | | | | |
| ingredients including | | | | | | | | | | | | |
| rifampicin | | | | | | | | | | | | |
| NaCl* | 150 | mM | 150 | mM | 150 | mM | 150 | mM | 0.90% | | 0.90% | |
| Tween 80* | 0.5% | | 0.5% | | 0.5% | | 0.5% | | 0.5% | | 0.5% | |
| EDTA* | 0.1% | | 0.1% | | 0.1% | | 0.1% | | 0.1% | | 0.1% | |
| Benzalkonium chloride* | 0.01% | | 0.01% | | 0.01% | | 0.01% | | 0.01% | | 0.01% | |
| Rifampicin* | 2.5 | mg/ml | 2.5 | mg/ml | 2.5 | mg/ml | 2.5 | mg/ml | 5 | mg/ml | 5 | mg/ml |

| | | | | |
|---|---|---|---|---|
| Sodium formaldehyde | 0.04 | mg/ml | 0.21 | mg/ml |

| | | | | |
|---|---|---|---|---|
| sulfoxylate dihydrate* | | | | |
| L-ascorbic acid* | 0.25% | 1% | 0.25% | 1% |

*Final Concentration

The buffer solutions, NaCl, Tween 80, and EDTA as listed in TABLE 3 were mixed with one another at room temperature in a beaker, and thereafter, rifampicin was added to the mixture and was completely dissolved in these liquid formulations at room temperature. A stock solution of sodium formaldehyde sulfoxylate dihydrate or L-ascorbic acid was added into each formulation, and the pH value of the formulation was then measured (Ex. 11A to Ex. 16A, inclusive). The formulations of Ex. 11A and Ex. 12A were stable at room temperature over more than several days, and no precipitates were generated therein. The formulations of Ex. 13A to Ex. 16A were stable in a refrigerator or a period of more than several days, and no precipitates were generated therein.

### Example 3: Topical Application of Rifamycin Compounds to the Retina

Rifampicin was delivered to the retina by application of topical eye drops. A 0.25% Rifampicin eye drop formulation was used in these studies. The eye drop formulation showed good delivery efficiency, and the retinal tissues obtained a micro gram concentration of rifampicin per g of the tissues by the eye drop application.

Four male Sprague-Dawley rats (250 to 300 g) were used to measure the retina exposure level of rifampicin after the application of eye drops. Two rats received 3 drops of the eye drop formulation (0.25% rifampicin), which is shown as Example 1A in TABLE 1, in each eye under isoflurane sedation. The remaining two rats received the same application, but 10 drops of the eye drops, in each eye under isoflurane sedation. A single drop contained 5 μL of the above-described formulation, and application of each drug drop was carried out with intervals of 30 minutes. After the eye drop application, the retina was excised from each rat under a dissection microscope. In addition, “non-treated” negative controls were used, and the retina was excised from two rats without performing any treatments on the rats. The retina was placed in a 1.5-ml microcentrifuge tube (1 retina/tube), and was fully washed with DPBS. After completion of the washing procedures, the retinal tissues in the microcentrifuge tube were frozen in dry ice, and were then preserved for quantification by LC/MS analysis. TABLE 4 shows quantification of rifampicin which was extracted from the retinal tissues. Rifampicin was delivered to the retina by the eye drop formulation (0.25% rifampicin) in a dose dependent manner.

TABLE 4
Amount of rifampicin delivered to retina by application
of topical eye drops (0.25% rifampicin)

| | Non-treated | 3 drops | 10 drops |
|---|---|---|---|
| Rifampicin extracted from retina | BDL | 1,288* | 2,939* |
| (Average of n = 3, ng/1 g of tissues) | | | |
| Standard deviation | N/A | 394 | 50.54 |

BDL: Below detection limit
*Evaluated by p value of 0.014

Retinal delivery of rifampicin by application of the topical eye drop formulation shown as Ex. 1A in TABLE 1 was greater than 100 times more efficient than dexamethasone retinal delivery. As depicted in TABLE 5, topical eye drop formulations, e.g., the formulation shown as Ex. 1A in TABLE 1, of the present disclosure surprisingly exhibit increased efficacy of retinal delivery of rifampicin, in comparison to other drugs.

TABLE 5

| Compound (brand name) | Drug amount | Drug amount | |
|---|---|---|---|
| MOA for indication or usage | administered | detected in retina | Reference |
| Betoptic 0.5% (MW: 307) | 0.5% | About 300 ng/1 g | Osborne et al., Exp |
| Beta 1 receptor blocker for | 4 drops | of retinal tissues | Eye Res. 1999 |
| glaucoma | (120-200 μL, | (Rabbit) | 69: 331-42. |
| | 600-1,000 μg) | | |
| Betoptic 0.5% (MW: 307) | 0.5% | About 480 ng/1 g | Osborne et al., Exp |
| Beta 1 receptor blocker for | 8 drops | of retinal tissues | Eye Res. 1999 |
| glaucoma | (240-400 μL, | (Rabbit) | 69: 331-42. |
| | 1200-2000 μg) | | |
| Dexamethasone (MW: 392) | 0.5% | About 33 ng/1 g | Sigurdsson et al., |
| Glucocorticoid for | 50 μL, 250 μg | of retinal tissues | Acta |
| inflammation | | (Rabbit) | Ophthalmologica |
| | | | Scandinavica. |
| | | | 2007 85: 598-602 |
| AMD101 | 0.25%, 3 drops | About 1,288 ng/1 g | Present study |
| | (15 μL, 37.5 μg) | of retinal tissues | |
| | | (Rat) | |
| AMD101 | 0.25%, 10 drops | About 2,939 ng/1 g | Present study |
| | (50 μL, 125 μg) | of retinal tissues | |
| | | (Rat) | |

### Example 4: Topical Application of Rifamycin Compounds to the Retina

The present example provides the experimental procedures and results of PK studies showing that rifampicin is delivered to the retina by subcutaneous (SC) injection. The detected amount of rifampicin delivered by SC was equivalent to the detected amount of rifampicin delivered by topical eye drops.

Six male Sprague-Dawley rats (250 to 300 g) were used to measure the retina exposure level of rifampicin after subcutaneous injection (20 mg/kg). A formulation, shown as Ex. 6A in TABLE 2, was used, and rifampicin was administered to the rats by SC. At time points of 1 hour, 3 hours, and 7 hours after the SC injection, the retinal tissues were excised from the rats under a dissection microscope. In addition, “non-treated” negative controls were used, and the retina was excised from two rats without performing any treatments on the rats. The retina was placed in a 1.5-ml microcentrifuge tube (1 retina/tube), and was fully washed with DPBS. After completion of the washing procedures, the retinal tissues in the microcentrifuge tube were frozen in dry ice, and were then preserved for quantification by LC/MS analysis. TABLE 6 shows quantification results of rifampicin, which was extracted from the retinal tissues. The amount of rifampicin detected in the retina, which was delivered by SC, was equivalent to the amount of rifampicin delivered by topical eye drops (see TABLE 3).

TABLE 6
Amount of rifampicin delivered to retina by
subcutaneous injection (20 mg/kg)

| | Non-treated | 1 hr | 3 hr | 7 hr |
|---|---|---|---|---|
| Rifampicin extracted | BDL | 2,655 | 3,864 | 2,516 |
| from retina | | | | |
| (Average of n = 4, ng/ | | | | |
| 1 g of tissues) | | | | |
| Standard deviation | N/A | 348 | 402 | 447 |

BDL: Below detection limit

### Example 5: Pharmacokinetic Studies Using a 0.25% Rifampicin Eye Drop Formulation

The present example provides the experimental procedures and results of PK studies using a 0.25% rifampicin eye drop formulation. Six male Sprague-Dawley rats (250 to 300 g) were used to measure the retina exposure level of rifampicin after application of the topical eye drops. An eye drop formulation (15 μL), which is shown as Ex. 1A in TABLE 1, was used, and the compound was administered to a single eye of each rat. The above-described formulation (15 μL) contained 37.5 μg of rifampicin. At time points of 1 hour, 3 hours, and 7 hours after the application of the eye drops, the retinal tissues were excised from the rats under a dissection microscope. In addition, “non-treated” negative controls were used, and the retina was excised from one rat without performing any treatments on the rats. The retina was placed in a 1.5-ml microcentrifuge tube (1 retina/tube), and was fully washed with DPBS. After completion of the washing procedures, the retinal tissues in the microcentrifuge tube were frozen in dry ice, and were then preserved for quantification by LC/MS analysis. TABLE 7 shows quantification of rifampicin which was extracted from the retinal tissues. Rifampicin was delivered to the retina by the eye drop formulation (0.25% rifampicin).

TABLE 7
Amount of rifampicin delivered to retina by application
of topical eye drops (0.25% rifampicin)

| | Non-treated | 1 hr | 3 hr | 7 hr |
|---|---|---|---|---|
| Rifampicin extracted from retina | BDL | 1,576 | 1,151 | 104 |
| (Average of n = 4, ng/1 g of tissues) | | | | |
| Standard deviation | N/A | 868 | 538 | 16 |

BDL: Below detection limit
Tmax: about 1 hr,
T½: about 3-4 hr

### Example 6: Dose Response Studies Using 0.25% and 0.5% Rifampicin Eye Drop Formulations

The present example provides the experimental procedures and results of dose response studies using 0.25% and 0.5% rifampicin eye drop formulations.

Four male Sprague-Dawley rats (250 to 300 g) were used to measure the retina exposure level of rifampicin after the application of topical eye drops. The eye drop formulations (15 μL each), which are shown as Ex. 1A and Ex. 6A listed in TABLE 1 and TABLE 2, were used, and the compounds were each applied to a single eye of each rat. The above-described formulations (15 μL each) contained 37.5 μg and 75 μg of rifampicin, respectively. One hour after the application of the eye drops, the retinal tissues were excised from the rats under a dissection microscope. In addition, “non-treated” negative controls were used, and the retina was excised from one rat without performing any treatments on the rats. The retina was placed in a 1.5-ml microcentrifuge tube (1 retina/tube), and was fully washed with DPBS. After completion of the washing procedures, the retinal tissues in the microcentrifuge tube were frozen in dry ice, and were then preserved for quantification by LC/MS analysis. TABLE 8 shows quantification of rifampicin, which was extracted from the retinal tissues. Rifampicin was delivered to the retina by application of the two eye drop formulations (0.25% and 0.5% rifampicin) in a dose dependent manner.

TABLE 8
Amount of rifampicin delivered to retina
by application of topical eye drops
(0.25% and 0.5% rifampicin)

| | | 0.25% | 0.5% |
|---|---|---|---|
| | Non- | rifampicin | rifampicin |
| | treated | 15 μL | 15 μL |
| Rifampicin extracted from retina | BDL | 1,576 | 6,068 |
| (Average of n = 4, ng/1 g of tissues) | | | |
| Standard deviation | N/A | 868 | 2,634 |

BDL: Below detection limit

### Example 7: Preclinical Efficacy

The present example provides the experimental procedures of a preclinical efficacy test performed on oxygen-induced retinopathy rat models, using a 0.25% rifampicin eye drop formulation.

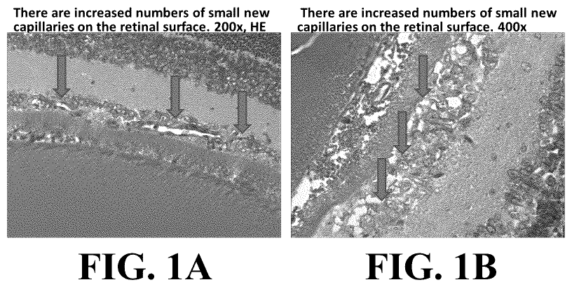

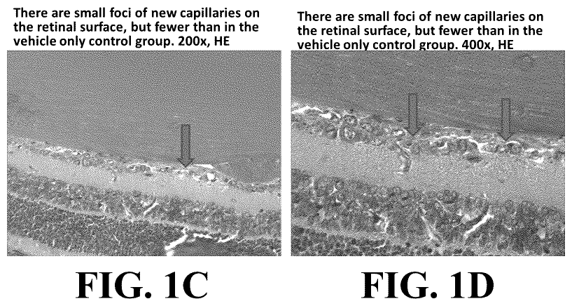

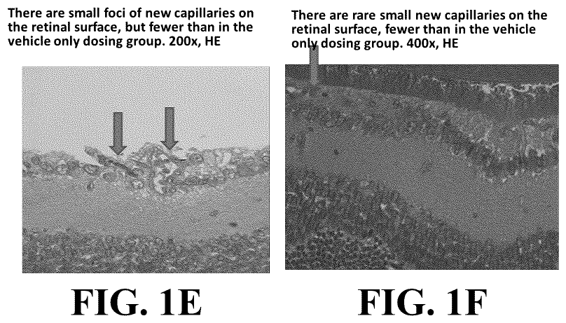

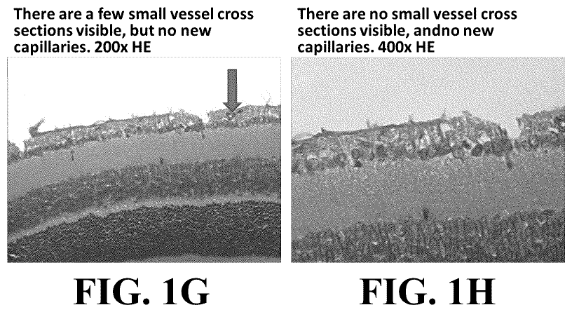

Oxygen-induced retinopathy rat models were produced according to the protocols of Yanni et al. (2010) and Dorfmann et al. (2008). Sprague-Dawley rat babies (and their nursing mothers) were exposed to a cycling oxygen environment (80% and 21%, about one day for each) for 15 days after starting from the day of birth (Day 0). On Day 15 (P15), the animals were moved to room air. Six babies, seven babies, and six babies were assigned to administration groups, namely, a control group of only vehicle, a rifampicin eye drop formulation administration group, and an SC injection administration group, respectively. An eye drop formulation shown as Ex. 1A in TABLE 1, or a vehicle only was administered to the eyes of the baby rats every day, for 5 days between P15 and P19, in the morning, about the noon, and in the evening. A formulation shown as Ex. 6A in TABLE 2 was used, and rifampicin was administered to the baby rats by SC injection at a dose of 20 mg/kg once a day from P15 to P19. On P20, all of the animals were euthanized, and the retina was visualized as histology sections. In those histology sections, capillaries in the retinal tissues in 3 eye balls (rifampicin topical application, rifampicin SC injection administration group, and non-induction of retinopathy) or 5 eye balls (control administration group of a vehicle only) were counted, so that neovascularization was quantified. These eye balls were selected from different animals in the above-described administration groups. Representative images of the histology sections are shown (see FIGS. 1A to 1H). The retina treated with the control of only vehicle showed an increased number of small new capillaries on the retinal surface (see FIGS. 1A and 1B). The retina treated with the AMD 101 topical eye drop formulation showed a small proliferation focus of new capillaries on the retinal surface, but the number of new capillaries was fewer than that in the control group (see FIGS. 1C and 1D). The retina treated with the rifampicin SC injection showed a small proliferation focus of new capillaries on the retinal surface, but the number of new capillaries is fewer than that in the control group (see FIGS. 1E and 1F). The retina, to which retinopathy was not induced, showed a few small vessel cross sections, but did not show any new capillaries (see FIGS. 1G and 1H). TABLE 9 shows quantification of capillaries detected in the histology sections.

TABLE 9
Neovascularization in retina of oxygen-induced retinopathy rat models
that was quantified by detection of capillaries in histological sections

| | Non-induction | | 0.25% | 0.5% |
|---|---|---|---|---|
| | of | Vehicle only | rifampicin | rifampicin |
| | retinopathy | control | topical eye drop | SC injection |
| | (n = 3) | (n = 5) | (n = 3) | (n = 3) |
| Average numbers of | 3.33 | 16.3 | 7.78 | 9.28 |
| capillaries in high power | | | | |
| field per eye | | | | |
| Standard deviation | 1.109721353 | 1.338468851 | 1.348295678 | 0.895463216 |

Vehicle only vs. 0.25% rifampicin eye drop: P value = 0.0058
Vehicle only vs. 0.5% rifampicin SC: P value = 0.0102

### Example 8: Delivery of Ophthalmic Formulations Comprising Rifamycin Compounds Via a Device

An ophthalmic formulation comprising an active ingredient, namely, rifampicin, rifabutin, rifapentine, rifalazil or rifaximin, and a pharmaceutically acceptable carrier, and optionally, additives as disclosed herein, is incorporated into an ocular solution used to immerse or wash contact lenses. A contact lens consisting of hydrophilic gel was optionally dried at ambient temperature, and was then immersed in a solution of a soaking agent or a swelling agent, containing an effective amount of the ophthalmic formulation, and was thus washed, or was immersed therein.

### Example 9: Oil or Water Based Formulations

Oil-based formulations (A-F; TABLE 10-1) comprising petrolatum and liquid paraffin mixed at various ratios were prepared, comprising sesame oil and light liquid paraffin. Rifampicin was formulated therein at 1% (w/w). TABLE 10-1 depicts the measured viscosities of the oil-based and water-based formulations (A-F) that were measured at 25° C. with various shear velocities measured using a Modular Compact Rheometer (MCR302, Anton Paar). A temperature control unit (P-PTD200) and a shaft (CP25-2) were used in these measurements. The temperature for each measurement was controlled and maintained at least in the range of 25.0±0.1° C.

TABLE 10-1

| | A | | B | | C | | D | | E | | F | |
|---|---|---|---|---|---|---|---|---|---|---|---|---|
| White | 99% | | 79.2% | | 49.5% | | — | | — | | — | |
| Petroleum | | | | | | | | | | | | |
| Liquid | — | | 19.8% | | 49.5% | | 99% | | — | | — | |
| Paraffin | | | | | | | | | | | | |
| Sesame Oil | — | | — | | — | | — | | 99% | | — | |
| Light Liquid | — | | — | | — | | — | | — | | 99% | |
| Paraffin | | | | | | | | | | | | |
| Viscosity | 3815 | mPaS | 2145 | mPaS | 867 | mPaS | 161 | mPaS | 63 | mPaS | 6 | mPaS |

| | | | | | | |
|---|---|---|---|---|---|---|
| Shear | 200 s⁻¹ | 200 s⁻¹ | 200 s⁻¹ | 200 s⁻¹ | 200 s⁻¹ | 200 s⁻¹ |
| Velocity s⁻¹ | | | | | | |

Water-based suspension formulations (G and H; TABLE 10-2) were prepared with NaCl in the presence or absence of a viscosity imparting agent (cellulose polymers), respectively. A water-based solubilized formulation (RK32) was prepared with polyoxyethylene castor oil, ethylene glycol monostearate, a viscosity imparting agent (cellulose polymers). Rifampicin was formulated therein at 1% (w/w). The pH of the formulations H and RK32 was adjusted to a pH of about 7.0 and 8.4 by using phosphate buffer, respectively. The pH of the formulation G will be adjusted to a pH of about 7.0 by using phosphate buffer. TABLE 10-2 depicts the measured viscosities of the water-based formulations (G, H, and RK32) at 25° C. with various shear velocities by using a Modular Compact Rheometer (MCR302, Anton Paar). In these measurements, a temperature control unit (P-PTD200) and a shaft (CP25-2) were used. During the measurements, the temperature was controlled and maintained at least in the range of 25.0±0.1° C.

TABLE 10-2

| | G | H | RK32 |
|---|---|---|---|
| NaCl | 0.67% | 0.4% | — |
| Polyoxyethylene | — | — | 5% |
| castor oil | | | |
| Ethylene glycol | — | — | 3% |
| monostearate | | | |
| Cellulose | 0.8% | — | 0.5% |
| polymers | | | |
| Na2HPO4 | 0.3% | 1.5% | 1.5% |
| EDTA | — | 0.1% | 0.1% |
| Anti-oxidants | — | 0.3% | 0.3% |
| pH | 7.1 | 7.0 | 8.3 |
| Viscosity | 101 mPaS | 1.3 mPaS | 39.7 mPaS |
| Shear Velocity s⁻¹ | 200 s⁻¹ | 200 s⁻¹ | 200 s⁻¹ |

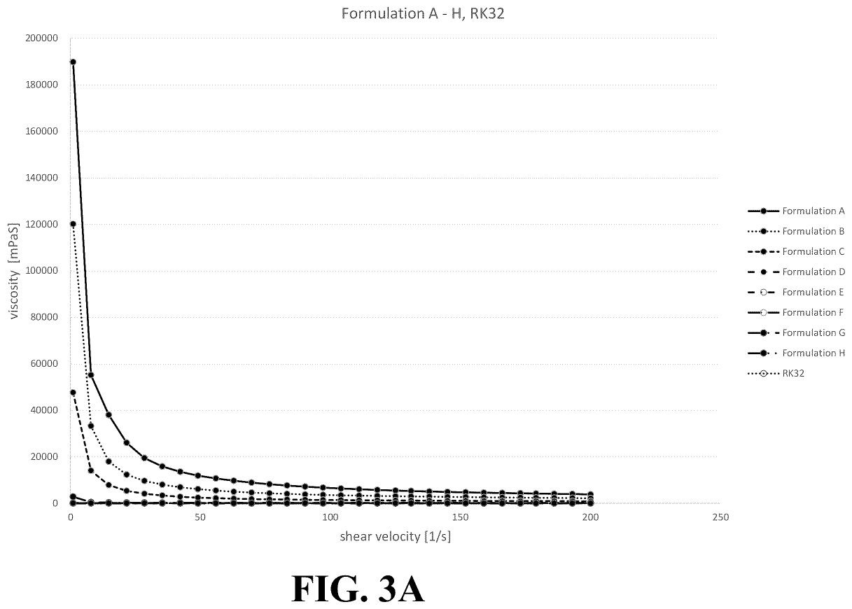

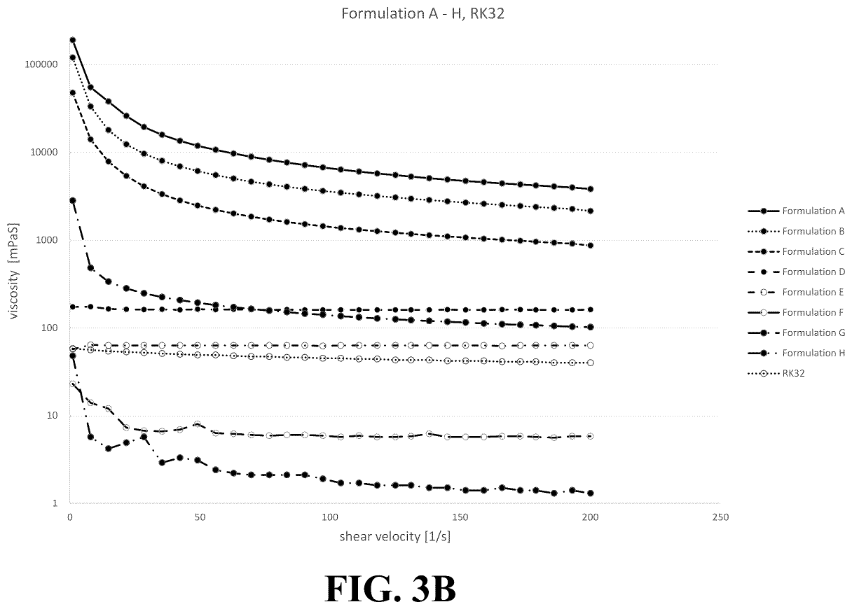

The relationship between viscosity and shear velocity was determined for oil-based formulations (A-F) and water-based formulations (G, H, and RK32) at 25° C. Shear is the relative motion between adjacent layers of a fluid. Shear velocity is the rate of change of velocity at which a fluid layer passes over another adjacent fluid layer. The relationship between viscosity (mPaS) and shear velocity (s⁻¹) for each formulation is depicted in FIG. 3A and FIG. 3B.

Oil-based formulations (E; TABLE 10-3) comprising petrolatum and liquid paraffin were prepared, and rifampicin was be formulated therein at 0.01% (w/w) and 0.001% (w/w). TABLE 10-3 describes the measured viscosities of the oil-based formulations that were measured at 25° C. with various shear velocities by using a Modular Compact Rheometer (MCR302, Anton Paar). A temperature control unit (P-PTD200) and a shaft (CP25-2) were used in these measurements. During the measurements, the temperature was controlled and maintained at least in the range of 25.0±0.1° C.

TABLE 10-3

| | C (0.01% | C (0.001% | E (0.1% | E (0.01% |
|---|---|---|---|---|
| | rifampicin) | rifampicin) | rifampicin) | rifampicin) |
| Viscosity | 801 mPaS | 781 mPaS | 62 mPaS | 65 mPaS |
| Shear | 200 s⁻¹ | 200 s⁻¹ | 200 s⁻¹ | 200 s⁻¹ |
| Velocity | | | | |

Oil-based formulations (A-G; TABLE 10) comprising petrolatum and liquid paraffin mixed at various ratios, sesame oil, dimethylpolysiloxane, and light liquid paraffin will be prepared, and rifampicin will be formulated therein at 1% (w/w), 0.01% (w/w), or 0.001% (w/w) at 37° C. Viscosities of the oil-based formulations (A-G) will be measured at 37° C.

### Example 10: Determination of Effective Dose of Rifampicin in Mouse Laser-Induced CNV Models

Eyes of C57BL/6J mice were laser irradiated, and CNV were induced in sub-retina tissues in the eyes. Vehicle only negative control or various concentrations of rifampicin dissolved in a water-based formulation were administered once daily by subcutaneous injections. In 7 days after the laser irradiation, a FITC-dextran solution was administered to the tail vein. The eyeballs were removed under euthanasia, and the removed eyeballs were fixed with 4% paraformaldehyde phosphate buffer. The cornea, iris and lens were excised, and retinal tissues other than retinal pigment epithelium cells were stripped using a microspacer. The optic cups were divided into 4 to 6 sections using a corneal microscissor, and they were placed on a slide glass. A confocal laser scanning microscope was used to photograph these fluorescent images that showed neovascularization induced in the sub-retina. The CNV areas were quantified using ImageJ (TABLES 11-1 and 11-2; Image software publicly available by NIH). Pixels that were derived from fluorescent CNV areas in eyes of the vehicle only negative control were evaluated as 100%, and percentages of fluorescent pixels from dosing groups administered by various concentrations of rifampicin were evaluated. P-values against negative control results obtained from the vehicle only dosing group were evaluated by the Williams multiple comparison test.

TABLE 11-1

| | | Rifampicin SC | Rifampicin SC |
|---|---|---|---|
| | Vehicle only SC | (0.2 mg/kg, | (0.7 mg/kg, |
| | (n = 10) | n = 10) | n = 10) |
| Average CNV area | 100 | 90.17 | 81.00 |
| (%) | | | |
| SE | 7.8957 | 5.4815 | 6.9349 |
| P-value (Williams | | >0.05 | 0.045 |
| multiple comparison | | | |
| test) | | | |

TABLE 11-2

| | | Rifampicin SC | Rifampicin SC |
|---|---|---|---|
| | Vehicle only SC | (0.7 mg/kg, | (2 mg/kg, |
| | (n = 10) | n = 10) | n = 10) |
| Average CNV area | 100 | 74.78 | 78.33 |
| (%) | | | |
| SE | 7.8957 | 5.0596 | 5.7329 |
| P-value (Williams | | 0.003 | 0.011 |
| multiple comparison | | | |
| test) | | | |

These results suggest that a SC injection by a dosage of 0.7 mg/kg significantly inhibited neovascularization in the mouse laser-induced CNV model. An effective systemic dosage of rifampicin to inhibit bacterial infection was previously evaluated at 20 mg/kg. Therefore, the effective dosage to inhibit neovascularization in the sub-retina in the CNV model was approximately 30 times smaller than that of the bacterial inhibition.

To evaluate the effective area under the curve (AUC) of rifampicin delivered to the mouse sub-retina, rifampicin was administered to C57BL/6J mice by SC injection at 0.7 mg/kg. This effective dosage and administration route was evaluated in dose escalation studies by using the mouse laser-induced CNV model. The AUC reflects the exposure to drug after administration of a dose of the drug and is expressed in hr·ng/mg tissue. The AUC is dependent on the dose administered as well as the rate of elimination of the drug from tissue. The AUC is directly proportional to the dose when the drug follows linear kinetics, and AUC is inversely proportional to the clearance of the drug. In 1 hr, 3 hr, 6 hr, and 18 hr after the SC injection, eyeballs were extracted. Tissue from the retina and tissue from the sclera+sub-retina complex were isolated and extracted. These tissues were immediately frozen in liquid nitrogen. The tissue was subjected to LC/MS analysis for quantitation. The detection limit was 0.25 ng/g tissue. The average rifampicin delivered to sclera+sub-retina complex (ng/mg tissue) is shown in TABLE 12.

TABLE 12

| | 1 hr | 3 hr | 6 hr | 18 hr |
|---|---|---|---|---|
| Ave. rifampicin | 0.03 | 0.03 | 0.02 | BDL |
| delivered to sclera + | | | | |
| sub-retina complex | | | | |
| (ng/mg tissue) | | | | |
| SE | 0.006478 | 0.005913 | 0.005334 | N/A |

The effective AUC was evaluated as approximately 0.27 (hr·ng/mg tissue). Therefore, the efficacy of rifampicin in inhibiting neovascularization is expected to be seen when at least or more than the effective AUC was delivered to the sub-retina tissue. In separate mouse pharmacokinetics studies by SC injections of rifampicin, delivery ratios between sub-retina and sclera tissues were evaluated as approximately 7:3, respectively. Therefore, approximately 70% of rifampicin delivered to the sub-retina+sclera complex was evaluated to be delivered to the sub-retina.

### Example 11: Ocular Tissue Delivery of Rifampicin Via Oil-Based, Water-Based Suspension, and Water-Solubilized Formulations by Topical Applications

Rabbits (Kbs:JW) were placed under anesthesia by injecting a mixture of ketamine/xylazine. Both 20 μL of Oil and water-based formulations (A-H; TABLE 10-1) and 50 μL of a water-based formulation (RK32) contained 1% rifampicin. The formulations were topically dosed to eyes of the rabbits. In 1 hr after the topical applications of the formulations (A-F, and H), blood samples were collected from the rabbits, and plasma fractions of the blood samples were saved in vials. In 1 hr and 18 hr after the topical applications of the formulations (A-F), the rabbits were euthanized, and eyeballs that were dosed the formulations were extracted. Eyeballs were dissected, and vitreous, retina, sub-retina, and sclera tissues were prepared and immediately frozen in liquid nitrogen. In 0.5 hr, 1 hr, 3 hr, and 6 hr after the topical application of the water solubilized formulation RK32, in 1 hr, 3 hr, 6 hr, and 18 hr after the topical applications of the oil-based and water-based suspension formulations (B, C, F, and H), and in 1 hr and 6 hr after the topical application of the water-based suspension formulation (G), plasma fractions of blood samples and eyeball tissues, including retina and sub-retina tissues, were collected and extracted. They were immediately frozen in liquid nitrogen. These eye tissues and plasma samples were subject to LC/MS analysis for quantification. Detection limit was 0.25 ng/g tissue or 0.25 ng/mL plasma.

An effective AUC was evaluated in mouse studies that showed CNV model efficacy and pharmacokinetic profiles in tissues containing sclera and sub-retina. The mouse effective AUC was compared with AUC values connecting time points between 1 hr and 18 hr with each average concentrations detected in retina and sub-retina that obtained in the rabbit studies using the oil-based formulations (A-F), the water-based suspension formulations (G and H), and the water-solubilized formulation (RK32).

TABLE 13-1
(Sub-retina)

| (n = 3, | | | | | |
|---|---|---|---|---|---|
| Average) | AUC by oil- | AUC by oil- | AUC by oil- | AUC by oil- | AUC by oil- |
| Mouse | based | based | based | based | based |
| effective AUC | formulation A | formulation B | formulation B | formulation C | formulation C |
| (hr · ng/mg | (hr · ng/mg | (hr · ng/mg | (hr · ng/mg | (hr · ng/mg | (hr · ng/mg |
| tissue) | tissue) | tissue) | tissue) | tissue) | tissue) |
| 0.27 | 0.96 | 2.11 | 1.37 | 2.68 | 2.18 |
| X1 | X4 | X8 | X5 | X10 | X8 |
| | | | | | AUC by water- |
| | | | AUC by water- | AUC by water- | based |
| AUC by oil- | AUC by oil- | AUC by oil- | based | based | solubilized |
| based | based | based | suspension | suspension | formulation |
| formulation D | formulation E | formulation F | formulation G | formulation H | RK32 |
| (hr · ng/mg | (hr · ng/mg | (hr · ng/mg | (hr · ng/mg | (hr · ng/mg | (hr · ng/mg |
| tissue) | tissue) | tissue) | tissue) | tissue) | tissue) |
| 4.40 | 8.77 | 0.62 | 1.82 | 1.33 | 2.05 |
| X16 | X32 | X2 | X7 | X5 | X8 |

| Formulation B | 1 hr | 3 hr | 6 hr | 18 hr |
|---|---|---|---|---|
| Ave. rifampicin delivered to | 0.103 | 0.280 | 0.064 | 0.006 |
| sub-retina (ng/mg tissue) | | | | |
| SE | 0.023 | 0.082 | 0.044 | 0.001 |

| Formulation C | 1 hr | 3 hr | 6 hr | 18 hr |
|---|---|---|---|---|
| Ave. rifampicin delivered to | 0.702 | 0.287 | 0.043 | 0.014 |
| sub-retina (ng/mg tissue) | | | | |
| SE | 0.117 | 0.139 | 0.003 | 0.010 |

| Formulation F | 1 hr | 3 hr | 6 hr | 18 hr |
|---|---|---|---|---|
| Ave. rifampicin delivered to | 0.121 | 0.104 | 0.020 | 0.005 |
| sub-retina (ng/mg tissue) | | | | |
| SE | 0.034 | 0.026 | 0.010 | 0.003 |

| Formulation H | 1 hr | 3 hr | 6 hr | 18 hr |
|---|---|---|---|---|
| Ave. rifampicin delivered to | 0.486 | 0.169 | 0.024 | 0.000 |
| sub-retina (ng/mg tissue) | | | | |
| SE | 0.162 | 0.027 | 0.015 | 0.000 |

| Formulation RK32 | 0.5 hr | 1 hr | 3 hr | 6 hr |
|---|---|---|---|---|
| Ave. rifampicin delivered to | 0.677 | 0.644 | 0.305 | 0.108 |
| sub-retina (ng/mg tissue) | | | | |
| SE | 0.188 | 0.177 | 0.084 | 0.032 |

TABLE 13-2
(Retina)

| (n = 3, | | | | | |
|---|---|---|---|---|---|
| Average) | AUC by oil- | AUC by oil- | AUC by oil- | AUC by oil- | AUC by oil- |
| Mouse | based | based | based | based | based |
| effective AUC | formulation A | formulation B | formulation B | formulation C | formulation C |
| (hr · ng/mg | (hr · ng/mg | (hr · ng/mg | (hr · ng/mg | (hr · ng/mg | (hr · ng/mg |
| tissue) | tissue) | tissue) | tissue) | tissue) | tissue) |
| 0.27 | 0.27 | 0.46 | 0.31 | 0.74 | 0.53 |
| X1 | X1 | X2 | X1 | X3 | X2 |
| | | | | | AUC by water- |
| | | | AUC by water- | AUC by water- | based |
| AUC by oil- | AUC by oil- | AUC by oil- | based | based | solubilized |
| based | based | based | suspension | suspension | formulation |
| formulation D | formulation E | formulation F | formulation G | formulation H | RK32 |
| (hr · ng/mg | (hr · ng/mg | (hr · ng/mg | (hr · ng/mg | (hr · ng/mg | (hr · ng/mg |
| tissue) | tissue) | tissue) | tissue) | tissue) | tissue) |
| 1.31 | 1.06 | 0.17 | 0.52 | 0.46 | 0.22 |
| X5 | X4 | X1 | X2 | X2 | X1 |

| Formulation B | 1 hr | 3 hr | 6 hr | 18 hr |
|---|---|---|---|---|
| Ave. rifampicin delivered to | 0.021 | 0.079 | 0.015 | 0.001 |
| retina (ng/mg tissue) | | | | |
| SE | 0.005 | 0.023 | 0.004 | 0.000 |

| Formulation C | 1 hr | 3 hr | 6 hr | 18 hr |
|---|---|---|---|---|
| Ave. rifampicin delivered to | 0.161 | 0.045 | 0.018 | 0.002 |
| retina (ng/mg tissue) | | | | |
| SE | 0.036 | 0.012 | 0.003 | 0.001 |

| Formulation F | 1 hr | 3 hr | 6 hr | 18 hr |
|---|---|---|---|---|
| Ave. rifampicin delivered to | 0.023 | 0.027 | 0.009 | 0.001 |
| retina (ng/mg tissue) | | | | |
| SE | 0.002 | 0.006 | 0.001 | 0.001 |

| Formulation H | 1 hr | 3 hr | 6 hr | 18 hr |
|---|---|---|---|---|
| Ave. rifampicin delivered to | 0.108 | 0.059 | 0.020 | 0.000 |
| retina (ng/mg tissue) | | | | |
| SE | 0.032 | 0.020 | 0.012 | 0.000 |

| Formulation RK32 | 0.5 hr | 1 hr | 3 hr | 6 hr |
|---|---|---|---|---|
| Ave. rifampicin delivered to | 0.103 | 0.078 | 0.024 | 0.009 |
| retina (ng/mg tissue) | | | | |
| SE | 0.029 | 0.022 | 0.004 | 0.001 |

These data suggested that all of the oil-based formulations (A-F), the water-based suspension formulation (G and H), and the water solubilized formulation (RK32) delivered to retina and sub-retina tissues rifampicin amounts equivalent to or larger than the effective AUC that was required for inhibition of neovascularization in mouse CNV models by topical applications. Therefore, when these oil or water based formulations containing 1% rifampicin are applied to eyes of animals or humans, neovascularization in sub-retina or retina tissues will be effectively inhibited. Thus, the formulations presented here will be utilized for treating posterior neovascular eye diseases, including AMD and diabetic retinopathy.

### Example 12: Relationships of AUC Values and Formulations' Viscosity in Pharmacokinetics Studies

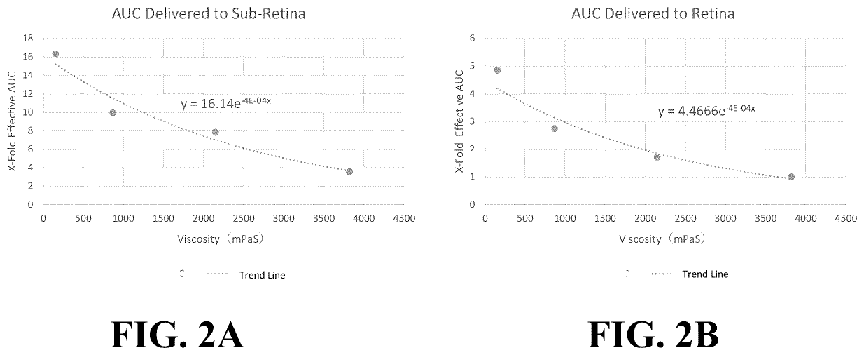

AUC values evaluated in sub-retina and retina tissues in pharmacokinetics studies (Example 11, TABLES 13-1 and 13-2) were analyzed to determine AUC correlations with the viscosities of Formulations A-F. The AUC (as X-Fold AUC) was plotted against the viscosity (mPaS) for each dose administered (FIG. 2A and FIG. 2B). In a range from approximately 100 mPaS to 4000 mPaS, a negative non-linear relationship with AUC values was found.

### Example 13: Ocular Tissue Delivery of Rifampicin Via Water-Based Suspension Formulations by Topical Applications

Rabbits (Kbs:JW) will be placed under anesthesia by injecting a mixture of ketamine/xylazine. 20 μL of a water-based suspension formulation (G; TABLE 10-2) will contain 1% rifampicin. The formulation will be topically dosed to eyes of the rabbits. In 1 hour after the topical applications of the formulations, blood samples will be collected from the rabbits, and plasma fractions of the blood samples will be saved in vials. In 1 hr, 3 hr, 6 hr, and 18 hr after the topical applications, the rabbits will be euthanized, and the eyeballs that will be dosed the formulations will be extracted. Eyeballs will be dissected, and vitreous, retina, sub-retina, and sclera tissues will be prepared and immediately frozen in liquid nitrogen. These eye tissues will be subject to LC/MS analysis for quantification. Detection limit will be 0.25 ng/g tissue or 0.25 ng/mL plasma.

Rifampicin prepared in a water-based formulation (G) will be able to be detected in sub-retina and retina tissues. An effective AUC was previously evaluated in mouse studies that showed CNV model efficacy and pharmacokinetic profiles in tissues containing sclera and sub-retina. The mouse effective AUC will be compared with AUC values connecting time-points between 1 hr and 18 hr with average concentrations detected in sub-retina and retina tissues that obtained in the rabbit studies using the water-based suspension formulations (G).

### Example 14: Oil-Based Formulation Plasma Exposure

In the pharmacokinetic studies described in Example 11, oil and water-based formulations (A-D, and RK32) containing 1% rifampicin were topically applied to eyes of the rabbits. In 1 hr after the topical application, blood samples were collected from the rabbits, and plasma fractions were prepared from the blood samples to evaluate systemic exposures of rifampicin. Rifampicin in the plasma fractions was quantified by LC/MS (TABLE 14).

TABLE 14
Plasma: Rifampicin concentrations (ng/mL plasma)
in 1 hr after the topical dosing
(Average: n = 3)

| | A | B | C | D | RK32 |
|---|---|---|---|---|---|
| Rifampicin | 0.70 | 0.47 | 1.59 | 1.96 | 10.24 |
| (ng/mL | | | | | |
| plasma) | | | | | |
| SE | 0.2144 | 0.0982 | 0.2764 | 0.3129 | 0.9431 |

Plasma exposures detected by dosing the oil-based formulations (A-D) were significantly smaller than that by dosing the water-based formulation (RK32). Improvements by the oil-formulations were evaluated in the plasma exposure.

### Example 14: Oil-Based Formulation Plasma Exposure

In the pharmacokinetic studies described in Example 11, oil-based formulations (E-H) containing 1% rifampicin was topically applied to eyes of the rabbits. To evaluate the systemic exposure of rifampicin, blood samples will be collected from the rabbits 1 hr after the topical application and plasma fractions will be prepared from the blood samples. Rifampicin amounts in the plasma fractions will be quantified by LC/MS.

### Example 15: Sub-Retina and Retina Delivery of Rifampicin Via Oil-Based Formulations

Rabbits (Kbs:JW) were placed under anesthesia by injecting a mixture of ketamine/xylazine. 20 μL of Oil-based formulations (C; TABLE 10-3) contained 0.01% and 0.001% rifampicin. 20 μL of Oil-based formulations (E; TABLE 10-3) contained 0.1% and 0.01% rifampicin. The formulations were topically dosed to eyes of the rabbits. The rabbits were euthanized 1 hr, 3 hr, 6 hr, and 18 hr after topical application of the Oil-based formulations C or E, and eyeballs that were dosed with the formulations were extracted. The eyeballs were dissected, and vitreous, retina, sub-retina, and sclera tissues were prepared and immediately frozen in liquid nitrogen. These eye tissues were subject to LC/MS analysis for quantification using a detection limit of 0.25 ng/g tissue or 0.25 ng/mL plasma (TABLE 15).

TABLE 15
(Average: n = 3)

| | Sub-retina | |
|---|---|---|
| | AUC by oil-based | AUC by oil-based |
| Mouse effective | formulation C | formulation C |
| AUC | 0.01% rifampicin | 0.001% rifampicin |
| (hr · ng/mg tissue) | (hr · ng/mg tissue) | (hr · ng/mg tissue) |
| 0.27 | 0.01 | 0.001 |
| X1 | X0.04 | X0.004 |
| | AUC by oil-based | AUC by oil-based |
| Mouse effective | formulation E | formulation E |
| AUC | 0.1% rifampicin | 0.01% rifampicin |
| (hr · ng/mg tissue) | (hr · ng/mg tissue) | (hr · ng/mg tissue) |
| 0.27 | 0.27 | 0.18 |
| X1 | X1 | X0.7 |

Neovascularization in sub-retina or retina tissues will be effectively inhibited when ocular topical applications of rifampicin formulations are applied to animal or human eyes in a concentration range of at least 1%-0.01%. Thus, the oil-based formulations of the present disclosure will be utilized for treating posterior neovascular eye diseases, including AMD and diabetic retinopathy.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to”, “at least”, “greater than”, “less than”, and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

We claim:

1. An ophthalmic composition having a viscosity of at least 1 mPaS at 25° C., the composition comprising an effective amount of one or more rifamycin compounds or a pharmaceutically acceptable salt thereof, a buffer solution, and optionally one or more viscosity imparting agents.

2. The composition of claim 1, wherein the one or more rifamycin compounds is selected from the group consisting of rifamycin SV, 3-formyl rifamycin SV, rifampicin, rifabutin, rifapentine, and rifaximin.

3. The composition of claim 1, wherein the one or more rifamycin compounds is rifampicin.

4. The composition of claim 3, wherein the effective amount of the one or more rifamycin compounds comprises a final concentration in the composition of at least 0.01% w/w, or at least 0.01 to at least 0.25% w/w, or at least 0.25 to at least 0.5% w/w, or 0.5 to at least 1.5% w/w, or at least 0.75 to at least 1.5% w/w, or at least 1 to at least 1.5% w/w, or at least 1.5% w/w.

5. The composition of claim 4, wherein the buffer solution is selected from the group consisting of: acetic acid, boric acid, citric acid, lactic acid, phosphoric acid, hydrochloric acid-potassium chloride, glycine, aconitic acid, citric acid-phosphoric acid, succinic acid, phthalic acid, maleic acid, cacodylic acid, tris (trishydroxymethylaminomethane), barbituric acid, borax, 2-amino-2-methyl-1,3-propanediol (Ammediol), sodium carbonate-sodium bicarbonate, HEPES (4-(2-hydroxyethyl)-1-piperazineethanesulfonic acid), ACES (N-(2-acetamido)-2-aminoethanesulfonic acid), ADA (N-(2-acetamido) iminodiacetic acid), BES (N,N-bis(2-hydroxyethyl)-2-aminoethanesulfonic acid), Bicine (N,N-bis(2-hydroxyethyl) glycine), Bis-Tris (bis(2-hydroxyethyl) iminotris(hydroxymethyl) methane), CAPS (N-cyclohexyl-3-aminopropanesulfonic acid), CAPSO (N-cyclohexyl-2-hydroxy-3-aminopropanesulfonic acid), CHES (N-cyclohexyl-2-aminoethanesulfonic acid), DIPSO (3-[N,N-bis(2-hydroxyethyl) amino]-2-hydroxypropanesulfonic acid), EPPS (3-[4-(2-hydroxyethyl)-1-piperazinyl] propanesulfonic acid), HEPES-Na (sodium 2-[4-(2-hydroxyethyl)-1-piperazinyl] ethanesulfonate), HEPPSO (2-hydroxy-3-[4-(2-hydroxyethyl)-1-piperazinyl] propanesulfonic acid, monohydrate), MES (2-morpholinoethanesulfonic acid, monohydrate), MOPS (3-morpholinopropanesulfonic acid), MOPSO (2-hydroxy-3-morpholinopropanesulfonic acid), PIPES (piperazine-1,4-bis(2-ethanesulfonic acid)), POPSO (piperazine-1,4-bis(2-hydroxy-3-propanesulfonic acid), dihydrate), TAPSO (2-hydroxy-N-tris(hydroxymethyl)methyl-3-aminopropanesulfonic acid), TES (N-tris(hydroxymethyl)methyl-2-aminoethanesulfonic acid), Tricine (N-[tris(hydroxymethyl)methyl]glycine), hydrochloric acid, sodium hydroxide, sodium phosphate, sodium borate, sodium citrate, sodium acetate, and sodium lactate; and buffer agents, such as citrate/dextrose, sodium bicarbonate, and ammonium chloride, and citrate, phosphate, borate, bicarbonate, sodium salt, and potassium.

6. The composition of claim 5, wherein the one or more viscosity imparting agents is selected from the group consisting of: petrolatum, liquid paraffin, light liquid paraffin, castor oil, mineral oil, cotton seed oil, soybean oil, sesame oil, cellulose polymers, corn oil, Petroleum resin, macrogol, glycerol, polybutene, rosin, polyvinyl alcohol, polystyrene, polyacrylic acid, propylene glycol, piperonyl butoxide, hypromellose, talc, gelatin, hydrogenated rosin glycerol ester, aliphatic hydrocarbon resin, benzyl acetate, copal resin, silicic acid, silicone, dimethylpolysiloxane, aluminum magnesium silicate, xanthan gum, sodium chondroitin sulfate, cyclodextrin, carboxyvinyl polymer, sodium alginate, propylene glycol alginate, carrageenan, carmellose sodium, gluconolactone, squalene, stearyl alcohol, aluminum stearate, lanolin, cetanol, gelatin, sorbitol, dextran, dextrin, tragacanth, palmitic acid, hyaluronate, hydroxyethylcellulose, hydroxyethylmethylcellulose, hydroxypropylcellulose, butylene glycol, polyoxyethylene polyoxypropylene glycol, polysorbate, sodium metaphosphate, methylcellulose, methyl vinyl ester maleic anhydride copolymer, locust bean gum, and cellulosic polymers.

7. The composition of claim 6, wherein the one or more viscosity imparting agents is petrolatum, liquid paraffin, light liquid paraffin, sesame oil, or cellulose polymers.

8. The composition of claim 7, wherein the composition comprises a final viscosity of at least about 0 mPaS to at least about 1 mPaS at 25° C., at least about 1 mPaS to at least about 5 mPaS at 25° C., at least about 5 mPaS to at least about 50 mPaS at 25° C., at least about 50 mPaS to at least about 100 mPaS at 25° C., at least about 100 mPaS to at least about 200 mPaS at 25° C., or optionally about 100 mPaS, or about 150 mPaS, or about 160 mPaS, or about 170 mPaS, or about 180 mPaS, or about 190 mPaS, or about 200 mPaS, at 25° C.

9. The composition of claim 8, wherein the composition comprises a final viscosity of at least about 161 mPaS at 25° C.

10. The composition of claim 9, wherein the composition comprises a final viscosity of at least about 500 mPaS to at least about 900 mPaS at 25° C., or optionally about 500 mPaS, about 600 mPaS, about 700 mPaS, about 800 mPaS, about 850 mPaS, about 860 mPaS, about 870 mPaS, about 880 mPaS, about 890 mPaS, or about 900 mPaS, at 25° C.

11. The composition of claim 10, wherein the composition comprises a final viscosity of at least about 781 mPaS at 25° C., at least about 801 mPaS at 25° cat least about 867 mPaS at 25° C.

12. The composition of claim 11, wherein the composition comprises a final viscosity of at least about 1000 mPaS to at least about 2500 mPaS at 25° C., or optionally about 1000 mPaS, about 1500 mPaS, about 2000 mPaS, about 2100 mPaS, about 2200 mPaS, about 2300 mPaS, about 2400 mPaS, or about 2500 mPaS, at 25° C.

13. The composition of claim 12, wherein the composition comprises a final viscosity of at least about 2145 mPaS, at 25° C.

14. The composition of claim 13, wherein the composition comprises a viscosity of at least about 3000 mPaS to at least about 4000 mPaS at 25° C., or optionally about 3000 mPaS, about 3500 mPaS, about 3600 mPaS, about 3700 mPaS, about 3800 mPaS, about 3900 mPaS, or about 4,000 mPaS, at 25° C.

15. The composition of claim 14, wherein the composition comprises a viscosity of at least about 3815 mPaS at 25° C.

16. The composition of any one of claims 8-15, wherein the one or more rifamycin compounds is rifampicin, wherein the one or more viscosity imparting agents is petrolatum, and wherein the effective amount of rifampicin comprises a final concentration in the composition of at least about 0.5% w/w.

17. The composition of any one of claims 8-15, wherein the one or more rifamycin compounds is rifampicin, wherein the one or more viscosity imparting agents is liquid paraffin, and wherein the effective amount of rifampicin comprises a final concentration in the composition of at least about 0.001% w/w to about 1% w/w.

18. The composition of any one of claims 8-15, wherein the one or more rifamycin compounds is rifampicin, wherein the one or more viscosity imparting agents is petrolatum, and wherein the effective amount of rifampicin comprises a final concentration in the composition of at least about 0.001% w/w to about 1% w/w.

19. The composition of any one of claims 8-15, wherein the one or more rifamycin compounds is rifampicin, wherein the one or more viscosity imparting agents is liquid paraffin, and wherein the effective amount of rifampicin comprises a final concentration in the composition of at least about 0.001% w/w to about 1% w/w.

20. The composition of any one of claims 1-15, wherein the one or more rifamycin compounds is rifampicin, wherein the one or more viscosity imparting agents is petrolatum, wherein the effective amount of rifampicin comprises a final concentration in the composition of at least about 0.001% w/w to about 1% w/w, and wherein the composition comprises a viscosity of at least about 3815 mPaS at 25° C.

21. The composition of any one of claims 1-15, wherein the one or more rifamycin compounds is rifampicin, wherein the one or more viscosity imparting agents comprises petrolatum and liquid paraffin, wherein the effective amount of rifampicin comprises a final concentration in the composition of at least about 0.001% w/w to about 1% w/w, and wherein the composition comprises a viscosity of at least about 2145 mPaS at 25° C.

22. The composition of any one of claims 1-15, wherein the one or more rifamycin compounds is rifampicin, wherein the one or more viscosity imparting agents comprises petrolatum and liquid paraffin, wherein the effective amount of rifampicin comprises a final concentration in the composition of at least about 0.001% w/w to about 1% w/w, and wherein the composition comprises a viscosity of at least about 867 mPaS at 25° C.

23. The composition of any one of claims 1-15, wherein the one or more rifamycin compounds is rifampicin, wherein the one or more viscosity imparting agents comprises liquid paraffin, wherein the effective amount of rifampicin comprises a final concentration in the composition of at least about 0.001% w/w to about 1% w/w, and wherein the composition comprises a viscosity of at least about 161 mPaS at 25° C.

24. The composition of any one of claims 1-15, wherein the one or more rifamycin compounds is rifampicin, wherein the one or more viscosity imparting agents comprises sesame oil, wherein the effective amount of rifampicin comprises a final concentration in the composition of at least about 0.001% w/w to about 1% w/w, and wherein the composition comprises a viscosity of at least about 59 mPaS at 25° C.

25. The composition of any one of claims 1-15, wherein the one or more rifamycin compounds is rifampicin, wherein the one or more viscosity imparting agents comprises light liquid paraffin, wherein the effective amount of rifampicin comprises a final concentration in the composition of at least about 0.001% w/w to about 1% w/w, and wherein the composition comprises a viscosity of at least about 5 mPaS at 25° C.

26. The composition of any one of claims 1-15, wherein the one or more rifamycin compounds is rifampicin, wherein the effective amount of rifampicin comprises a final concentration in the composition of at least about 0.001% w/w to about 1% w/w, and wherein the composition comprises a viscosity of at least about 1 mPaS at 25° C.

27. The composition of any one of claims 1-5, wherein the one or more rifamycin compounds is rifampicin, wherein the effective amount of rifampicin comprises a final concentration in the composition of at least about 0.001% w/w to about 1% w/w, and wherein the composition comprises a viscosity of at least about 1 mPaS at 25° C., and wherein the composition does not comprise a viscosity imparting agent.

28. The composition of any one of claims 1-15, wherein the one or more rifamycin compounds is rifampicin, wherein the one or more viscosity imparting agents comprises cellulose polymers, wherein the effective amount of rifampicin comprises a final concentration in the composition of at least about 0.001% w/w to about 1% w/w, and wherein the composition comprises a viscosity of at least about 38 mPaS at 25° C.

29. The composition of any one of claims 1-15, wherein the one or more rifamycin compounds is rifampicin, wherein the one or more viscosity imparting agents comprises cellulose polymers, wherein the effective amount of rifampicin comprises a final concentration in the composition of at least about 0.001% w/w to about 1% w/w, and wherein the composition comprises a viscosity of at least about 290 mPaS at 25° C.

30. The composition of any one of claims 1-29, wherein the effective amount of one or more rifamycin compounds or a pharmaceutically acceptable salt thereof is the only therapeutically effective compound in the composition.

31. A method for delivering one or more rifamycin compounds to the sub retina, sclera, retina, and/or vitreous tissues, the method comprising topically administering the composition of any one of claims 1-30 to the eye.

32. A method for treating a neovascular eye disease, the method comprising topically administering the composition of any one of claims 1-30 to the eye.

33. The method of claim 31 or claim 32, wherein the composition is administered topically in a single dose per eye.

34. The method of claim 33, wherein the volume of the composition administered in a single dose per eye is in the range of about 2 μL to about 60 μL.

35. The method of claim 33 or claim 34, wherein the volume of the composition administered in a single dose per eye is in the range of about 10 μL to about 50 μL.

36. The method of any one of claims 33-35, wherein the volume of the composition administered in a single dose per eye is in the range of about 20 μL to 30 μL.

37. A method for delivering one or more rifamycin compounds to the sub retina, sclera, retina, and/or vitreous tissues, the method comprising systemically administering the composition of any one of claims 1-30.

38. A method for treating a neovascular eye disease, the method comprising systemically administering the composition of any one of claims 1-30.

39. The method of claim 37 or claim 38, wherein the composition is administered parenterally.

40. The method of any one of claims 37-39, wherein the composition is administered via intravitreal injection.

41. The method of claim 37 or claim 38, wherein the composition is administered via oral administration, infusion, implantation, subcutaneous injection, intravenous injection, or intramuscular injection.

42. The method of any one of claims 33-41, wherein the composition is administered at an effective dose of one or more rifamycin compounds of at least 0.2 mg/kg, at least 0.7 mg/kg, at least 2 mg/kg, or at least 20 mg/kg.

43. The method of claim 42, wherein the composition is administered at an effective dose of one or more rifamycin compounds of at least 0.7 mg/kg.

44. The method of any one of claims 31-43, wherein the composition is administered at an interval of at least 1 day, at least 2 days, at least 7 days, at least 14 days, at least 21 days, at least 28 days, at least 35 days, at least 42 days, at least 49 days, at least 56 days, or at least 64 days.

45. The method of any one of claims 31-44, wherein administration of the composition results in delivery of the one or more rifamycin compounds to the sub-retina, sclera, retina, and/or vitreous tissues.

46. The method of claim 45, wherein administration of the composition results in delivery of the one or more rifamycin compounds to the sub-retina and sclera.

47. The method of any one of claims 31-46, wherein administration of the composition inhibits neovascularization in sub-retina tissues.

48. The method of any one of claims 31-47, wherein administration of the composition results in at least about a 5-fold, 10-fold, 50-fold, or 100-fold reduction in plasma exposure of the one or more rifamycin compounds relative to oral dosing at 300 mg.

49. The method of claim 48, wherein administration of the composition results in at least about 100-fold reduction in plasma exposure of the one or more rifamycin compounds relative to oral dosing at 300 mg.

50. The method of any one of claims 31-36, wherein topical administration of the composition results in at least about a 5-fold, 10-fold, 50-fold, or 100-fold reduction in plasma exposure of the one or more rifamycin compounds relative to oral dosing at 300 mg.

51. The method of claim 50, wherein topical administration of the composition results in at least about 100-fold reduction in plasma exposure of the one or more rifamycin compounds relative to oral dosing at 300 mg.

52. The method of any one of claims 32-36 and 38-51, wherein the neovascular eye disease is selected from the group consisting of macular degeneration, diabetic retinopathy, chronic glaucoma, retinal detachment, sickle cell retinopathy, age-related macular degeneration (AMD), retinal ganglion cell injury, rubeosis iritis, inflammatory disease, chronic uveitis, neoplasm, Fuchs' heterochromic iridocyclitis, neovascular glaucoma, corneal neovascularization, choroidal neovascularization, retinal neovascularization, retinal angiomatous proliferation, glaucoma, glaucoma surgery, tissue adhesion, cicatrization, tissue fibrosis, and brain damage.

53. The method of claim 52, wherein the neovascular eye disease is AMD.

54. The method of claim 52, wherein the neovascular eye disease is dry AMD.

55. The method of claim 52, wherein the neovascular eye disease is wet AMD.

56. The method of any one of claims 53-55, wherein administration of the composition treats or prevents one or more complications of macular degeneration including, druse deposition/accumulation, macular edema, and neovacuolization.

57. The method of any one of claims 53-56, wherein the method further comprises administering one or more supportive therapies comprising administering a VEGF antagonist, surgery, laser therapy, photodynamic therapy, and/or dietary supplements for treating macular degeneration.

58. The method of claim 52, wherein the neovascular eye disease is diabetic retinopathy.

59. The method of claim 58, wherein the diabetic retinopathy is nonproliferative diabetic retinopathy (NPDR).

60. The method of claim 59, wherein the diabetic retinopathy is proliferative diabetic retinopathy (PDR).

61. The method of any one of claims 58-60, wherein the diabetic retinopathy involves diabetic macular edema (DME).

62. The method of claim 58, wherein administration of the composition treats or prevents one or more complications of diabetic comprising vitreous hemorrhage, retinal detachment, glaucoma, blindness, blurred vision, fluctuating vision, and/or macular edema.

63. The method of any one of claims 58-62, wherein the method further comprises administering one or more supportive therapies comprising administering a VEGF antagonist, surgery, laser therapy, photodynamic therapy, and/or dietary supplements for treating macular degeneration.

64. The method of any one of claims 31-63, wherein administration of the composition improves vision, increases visual acuity, and/or increases visual field.
